(19) United States
(12) Patent Application Publication
(10) Pub. No.: US 2013/0310332 A1
(43) Pub. Date: Nov. 21, 2013

(54) MAPLE TREE-DERIVED PRODUCTS AND USES THEREOF

(71) Applicants: Julie Barbeau, Boucherville (CA); Genevieve Beland, St-Hyacinthe (CA); Navindra P. Seeram, Charlestown, RI (US); Tao Yuan, Kingston, RI (US)

(72) Inventors: Julie Barbeau, Boucherville (CA); Genevieve Beland, St-Hyacinthe (CA); Navindra P. Seeram, Charlestown, RI (US); Tao Yuan, Kingston, RI (US)

(73) Assignees: UNIVERSITY OF RHODE ISLAND, Kingston, RI (US); FEDERATION DES PRODUCTEURS ACERICOLES DU QUEBEC, Longueuil, QC (CA)

(21) Appl. No.: 13/881,720

(22) PCT Filed: Oct. 14, 2011
(86) PCT No.: PCT/CA2011/001164

§ 371 Date: Aug. 7, 2013

Related U.S. Application Data
(60) Provisional application No. 61/406,290, filed on Oct. 25, 2010.
(60) Provisional application No. 61/449,333, filed on Mar. 4, 2011.

(51) Int. Cl. C07H 15/26; C07H 15/203; C07D 309/10

(52) U.S. Cl. CPC C07H 15/26; C07D 309/10; C07H 15/203
USPC 514/35; 549/417; 514/460; 536/18.2; 536/4.1; 536/18.1

(57) ABSTRACT

The present document describes a neutraceutical, cosmeceuticals, functional food, pharmaceutical, food ingredient, and non-food ingredient compositions comprising sugar maple extract, essential oil compositions comprising oil extracted from an Acer tree, sweetening compositions containing sugar extracted from maple tree leaves, food ingredients comprising maple tree extract, cosmetic composition comprising maple tree extracts, infusion compositions prepared from maple tree leaves, maple roots, maple wood, maple stems of leaves and samara, and stems/twigs as well as compounds isolated from sugar maple biomass and the methods of extracting the same.

### CROSS-REFERENCE TO RELATED APPLICATIONS

This application claims priority from U.S. provisional patent applications 61/406,290, filed Oct. 25, 2011, and 61/449,333, filed Mar. 4, 2011, the specifications of which is hereby incorporated by reference.

### BACKGROUND

(a) Field

The subject matter disclosed generally relates to products derived from harwood trees, especially any variety of maple tree, such as sugar maple (Acer Saccharum) or red maple (Acer Rubrum L.), and more specifically to neutraceutical compositions comprising maple tree extract, essential oil compositions comprising oil extracted from sugar maple or other maple, sweetening compositions containing sugar extracted from sugar maple, infusion compositions prepared from maple tree leaves, food ingredients comprising maple tree extract, cosmetic composition comprising maple tree extracts as well as compounds isolated from sugar maple biomass and the methods of extracting the same.

(b) Related Prior Art

Products derived from the sap of the sugar maple tree Acer Saccharum or other maple trees are well known in the art. For many years, it has been known that the sap of the hard or sugar maple trees, known as winter sap, could be concentrated very substantially from its original, very watery and only slightly sweet condition to well known maple syrup which, when further boiled, finally reaches crystalline stage wherein the product is maple sugar. For example, U.S. Pat. No. 3,397,062 to Nessly describes carbonated beverages derived from concentrated maple sap to which flavouring may be added. Also, U.S. Pat. No. 5,424,089 to Munch et al. describes carbonated beverages prepared from non-concentrated maple sap to which flavouring ingredients may have been added.

Other products concern principally maple sugar tree products such as maple syrup. For example U.S. Pat. No. 6,485,763 to Jampen describes a method for producing a shelf stable, spreadable high viscosity maple syrup by adding a sucrose-cleaving enzyme.

Using maple based products appears to present several advantages for a healthy diet, as maple-sugar products or products derived from any other variety of maple tree which contain molecules such as polyphenols as well as other nutrients such as vitamins and oligoelements that can contribute to good health. However, very little is known concerning the potential health benefits of other types of maple-based products such as products derived from the sap, the samara (including the fruits, the seeds as well as the stem), leaves (including the stem), twigs, roots, heartwood and sap wood, whole branches, wood from branches, bark from branches and bark of any Acer tree. Also, maple-derived sugar remains a relatively little known nutrient from which only recently has it been found potential properties as an anti-oxidant, anticancer, antibacterial, anti-diabetic agent, anti-inflammatory, anti-arthritic, anti-hyperglycemic, as well as beneficial effects on cardiovascular health, neurodegenerative diseases, Alzheimer's diseases, liver disorders (such as metabolic syndrome, damaged hepatic function, hepatic and liver dyslipidemia, hepatitis, liver cancer), atherosclerosis, hypertension, and skin diseases (such as eczema, psoriasis and the likes).

Therefore, there is a need for maple tree-derived products to improve or enhance health and well-being.

Therefore, there is a need for maple tree-derived sugar products to improve or enhance health and well-being

### SUMMARY

According to an embodiment, there is provided a composition for the prophylaxis of an ailment comprising a therapeutically effective amount of an extract of an Acer tree in association with a pharmaceutically acceptable carrier.

The extract of Acer tree may be an extract from a non-concentrated or concentrated sap, a samara fruit, a samara seed, a stems of leaf, a stem of a samara, a twig, a root, a leaf, a bark, a heartwood, a sapwood, a whole branch, a bark of a branch, a wood of a branch, a sugar, a syrup, a syrup extract, a syrup-derived product, a rejection of syrup or syrup-derived product production, a residue of syrup or syrup-derived product production, or combinations thereof.

The Acer tree may be chosen from Acer nigrum, Acer lanum, Acer acuminatum, Acer albopurpurascens, Acer argutum, Acer barbinerve, Acer buergerianum, Acer caesium, Acer campbeffii, Acer campestre, Acer capillipes, Acer cappadocicum, Acer carpinifolium, Acer caudatifolium, Acer caudatum, Acer cinnamomifolium, Acer circinatum, Acer cissifolium, Acer crassum, Acer crataegifolium, Acer davidii, Acer decandrum, Acer diabolicum, Acer distylum, Acer divergens, Acer erianthum, Acer etythranthum, Acer fabri, Acer garrettii, Acer glabrum, Acer grandidentatum, Acer griseum, Acer heldreichii, Acer hentyi, Acer hyrcanum, Acer ibericum, Acer japonicum, Acer kungshanense, Acer kweilinense, Acer laevigatum, Acer laurinum, Acer lobelii, Acer lucidum, Acer macrophyllum, Acer mandshuricum, Acer maximowiczianum, Acer miaoshanicum, Acer micranthum, Acer miyabei, Acer mono, Acer mono×Acer truncatum, Acer monspessulanum, Acer negundo, Acer ningpoense, Acer nipponicum, Acer oblongum, Acer obtusifolium, Acer oliverianum, Acer opalus, Acer palmatum, Acer paxii, Acer pectinatum, Acer pensylvanicum, Acer pentaphyllum, Acer pentapomicum, Acer pictum, Acer pilosum, Acer platanoides, Acer poliophyllum, Acer pseudoplatanus, Acer pseudosieboldianum, Acer pubinerve, Acer pycnanthum, Acer rubrum, Acer rufinerve, Acer saccharinum, Acer saccharum, Acer sempervirens, Acer shirasawanum, Acer sieboldianum, Acer sinopurpurescens, Acer spicatum, Acer stachyophyllum, Acer sterculiaceum, Acer takesimense, Acer tataricum, Acer tegmentosum, Acer tenuifolium, Acer tetramerum, Acer trautvetteri, Acer triflorum, Acer truncatum, Acer tschonoskii, Acer turcomanicum, Acer ukurunduense, Acer velutinum, Acer wardii, Acer x peronai, and Acer x pseudoheldreichii.

The Acer tree may be chosen from Acer Saccharum and Acer Rubrum L.

The syrup-derived product may comprise butter, granulated sugar, hardened sugar, soft sugar, taffy, flakes an extract from lyophilisation of a sap, a maple concentrate or a maple syrup, an extract from drying of a sap, a maple concentrate or a maple syrup, an extract from crystallization of a sap, a maple concentrate or a maple syrup, an extract from pulverization of a sap, a maple concentrate or a maple syrup, an extract from atomization of a sap, a maple concentrate or a maple syrup, an extract from centrifugation of a sap, a maple concentrate or a maple syrup, or combinations thereof.

The syrup extract may be chosen from a methanol extract, a butanol extract, a butanol extract with sugar, a butanol extract without sugar, an ethyl acetate extract, an ethanol extract, a 95% ethanol/5% hot water extract, or combinations thereof.

The extract of Acer tree may comprise an extract from concentrated Acer tree water issued from reverse osmosis, a concentrated Acer tree water issued from pre-boiling nanofiltration, a pasteurized Acer tree water, a sterilized Acer tree water, an Acer tree water issued from a high pressure processing, an Acer tree water sterilized by UV irradiation, an Acer tree water sterilized by microwave irradiation or combinations thereof.

The extract of Acer tree may comprise an Acer tree molecule.

The Acer tree molecule may comprise:
Lyoniresinol,
Isolariciresinol,
secoisolariciresinol,
Dehydroconiferyl alcohol,
5′-methoxy-dehydroconiferyl alcohol,
erythro-guaiacylglycerol-β-O-4′-coniferyl alcohol,
erythro-guaiacylglycerol-β-O-4′-dihydroconiferyl alcohol,
[3-[4-[(6-deoxy-α-L-mannopyranosyl)oxy]-3-methoxyphenyl]methyl]-5-(3,4-dimethoxyphenyl)dihydro-3-hydroxy-4-(hydroxymethyl)-2(3H)-furanone,
5-(3″,4″-dimethoxyphenyl)-3-hydroxy-3-(4′-hydroxy-3′-methoxybenzyl)-4-hydroxymethyl-dihydrofuran-2-one,
Scopoletin,
Fraxetin,
Isofraxidin,
Gallic acid,
Ginnalin A (acertannin),
Syringic acid,
Ginnalin B,
Ginnalin C,
Trimethyl gallic acid methyl ester
(E)-3,3′-dimethoxy-4,4′-dihydroxy stilbene,
p-coumaric acid,
Ferulic acid,
(E)-Coniferol,
Syringenin,
Dihydroconiferyl alcohol,
C-veratroylglycol,
2,3-dihydroxy-1-(3,4-dihydroxyphenyl)-1-propanone
2,3-Dihydroxy-1-(4-hydroxy-3,5-dimethoxyphenyl)-1-propanone,
3-Hydroxy-1-(4-hydroxy-3,5-dimethoxyphenyl)propan-1-one,
3′,4′,5′-Trihydroxyacetophenone,
4-Acetylcatechol,
2,4,5-Trihydroxyacetophenone,
1-(2,3,4-trihydroxy-5-methylphenyl)-ethanone,
2-Hydroxy-3′,4′-dihydroxyacetophenone,
Vanillin,
Syringaldehyde,
Catechaldehyde,
3,4-Dihydroxy-2-methylbenzaldehyde,
Catechol,
Catechin,
Epicatechin,
Quebecol,
(erythro, erythro)-1-[4-[2-hydroxy-2-(4-hydroxy-3-methoxyphenyl)-1-(hydroxymethyl)ethoxy]-3,5-dimethoxyphenyl]-1,2,3-propanetriol,
(erythro, threo)-1-[4-[2-hydroxy-2-(4-hydroxy-3-methoxyphenyl)-1-(hydroxymethyl)ethoxy]-3,5-dimethoxyphenyl]-1,2,3-propanetriol,
(threo, etythro)-1-[4-[(2-hydroxy-2-(4-hydroxy-3-methoxyphenyl)-1-(hydroxymethyl)ethoxy]-3-methoxyphenyl]-1,2,3-propanetriol,
(threo, threo)-1-[4-[(2-hydroxy-2-(4-hydroxy-3-methoxyphenyl)-1-(hydroxymethyl)ethoxy]-3-methoxyphenyl]-1,2,3-propanetriol,
threo-guaiacylglycerol-β-O-4′-dihydroconiferyl alcohol,
erythro-1-(4-hydroxy-3-methoxyphenyl)-2-[4-(3-hydroxypropyl)-2,6-dimethoxyphenoxy]-1,3-propanediol,
2-[4-[2,3-dihydro-3-(hydroxymethyl)-5-(3-hydroxypropyl)-7-methoxy-2-benzofuranyl]-2,6-dimethoxyphenoxy]-1-(4-hydroxy-3-methoxyphenyl)-1,3-propanediol,
Acerkinol,
Leptolepisol D,
Buddlenol E,
(1S,2R)-2-[2,6-dimethoxy-4-[(1S,3aR,4S,6aR)-tetrahydro-4-(4-hydroxy-3,5-dimethoxyphenyl)-1H,3H-furo[3,4-c]furan-1-yl]phenoxy]-1-(4-hydroxy-3-methoxyphenyl)-1,3-propanediol,
Syringaresinol,
Icariside E4,
Sakuraresinol,
1,2-diguaiacyl-1,3-propanediol
protocatechuic acid,
4-(dimethoxymethyl)-pyrocatechol,
Tyrosol,
4-hydroxycatechol,
Phaseic acid,
Nortrachelogenin 8′-O-β-D-glucopyranoside,
3-O-galloyl-1,5-anhydro-D-glucitol,
4-O-galloyl-1,5-anhydro-D-glucitol,
2,4-di-O-galloyl-1,5-anhydro-D-glucitol,
Quercetin-3-O-α-rhamnopyranoside,
2,3-di-O-galloyl-1,5-anhydro-D-glucitol,
3-Methoxy-4-hydroxyphenol 1-O-β-D-(6′-O-galloyl)-glucopyranoside,
3,5-Dihydroxy-4-methoxybenzoic acid methyl ester,
7,8-Dihydroxy-6-methoxycoumarin,
4-Methoxy-3,5-dihydroxybenzoic acid,
Methyl 3,5-dimethoxy-4-hydroxybenzoate,
Methyl vanillate,
Methyl gallate,
3,6-di-O-galloyl-1,5-anhydro-D-glucitol,
(+)-Epicatechin-(4β-8,2β-O-7)-catechin,
Epicatechin gallate,
(+)-Epicatechin-(4β-8,2β-O-7)-epicatechin,
Quercetin-3-O-(3″-O-galloyl)-α-rhamnopyranoside,
Quercetin-3-O-(2″-O-galloyl)-α-rhamnopyranoside,
2,4,6-tri-O-galloyl-1,5-anhydro-D-glucitol,

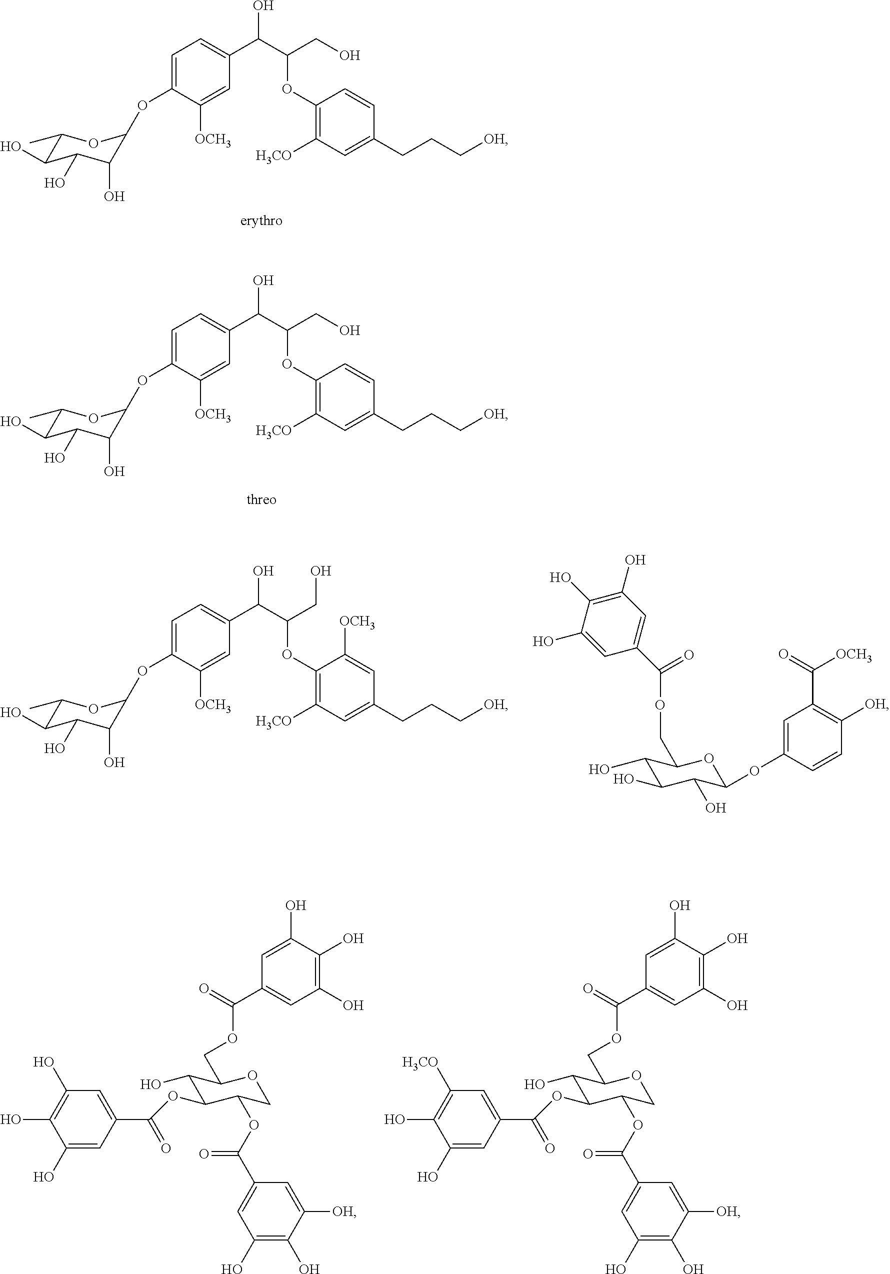 [SMILES: COc1cc(CCCO)ccc1OC(CO)C(O)c1ccc(OC2OC(C)C(O)C(O)C2O)c(OC)c1 ; COc1cc(CCCO)ccc1OC(CO)C(O)c1ccc(OC2OC(C)C(O)C(O)C2O)c(OC)c1 ; COc1cc(C(O)C(CO)Oc2c(OC)cc(CCCO)cc2OC)ccc1OC1OC(C)C(O)C(O)C1O ; COC(=O)c1cc(OC2OC(COC(=O)c3cc(O)c(O)c(O)c3)C(O)C(O)C2O)ccc1O ; O=C(OCC1OCC(OC(=O)c2cc(O)c(O)c(O)c2)C(OC(=O)c2cc(O)c(O)c(O)c2)C1O)c1cc(O)c(O)c(O)c1 ; COc1cc(C(=O)OC2C(OC(=O)c3cc(O)c(O)c(O)c3)COC(COC(=O)c3cc(O)c(O)c(O)c3)C2O)cc(O)c1O]

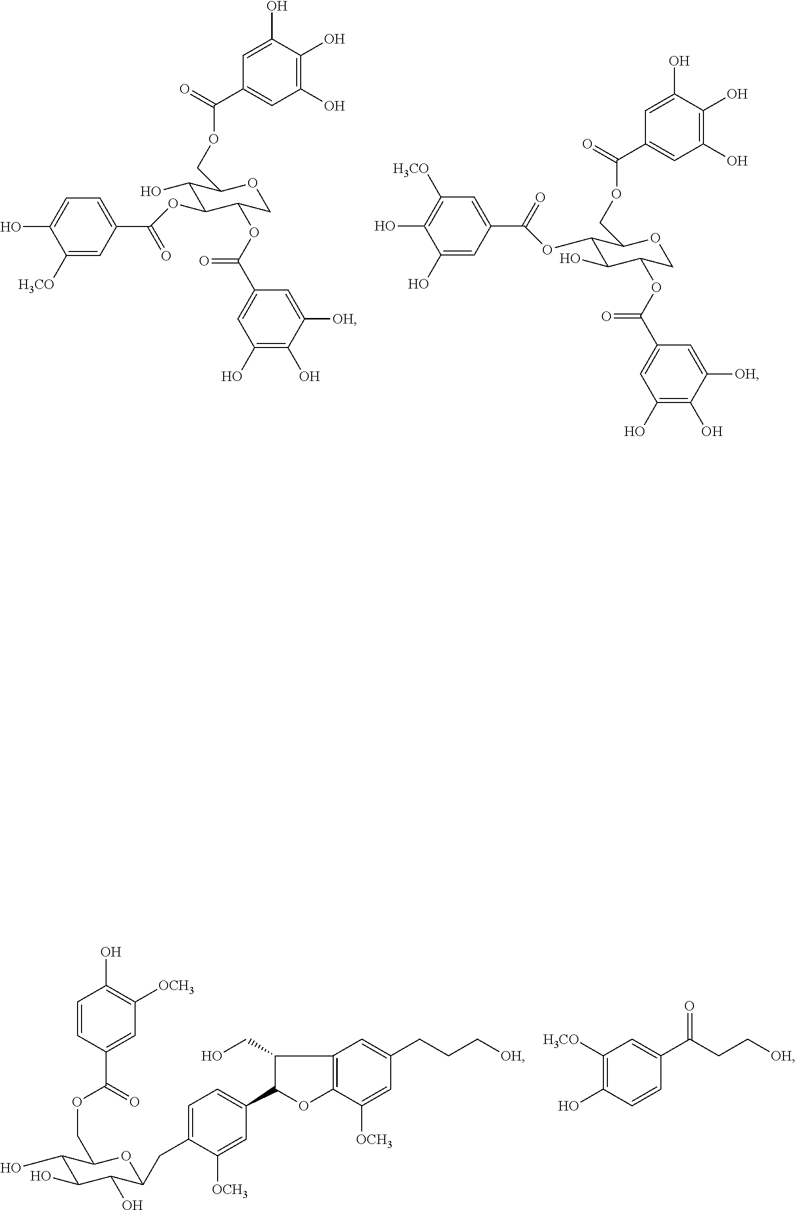

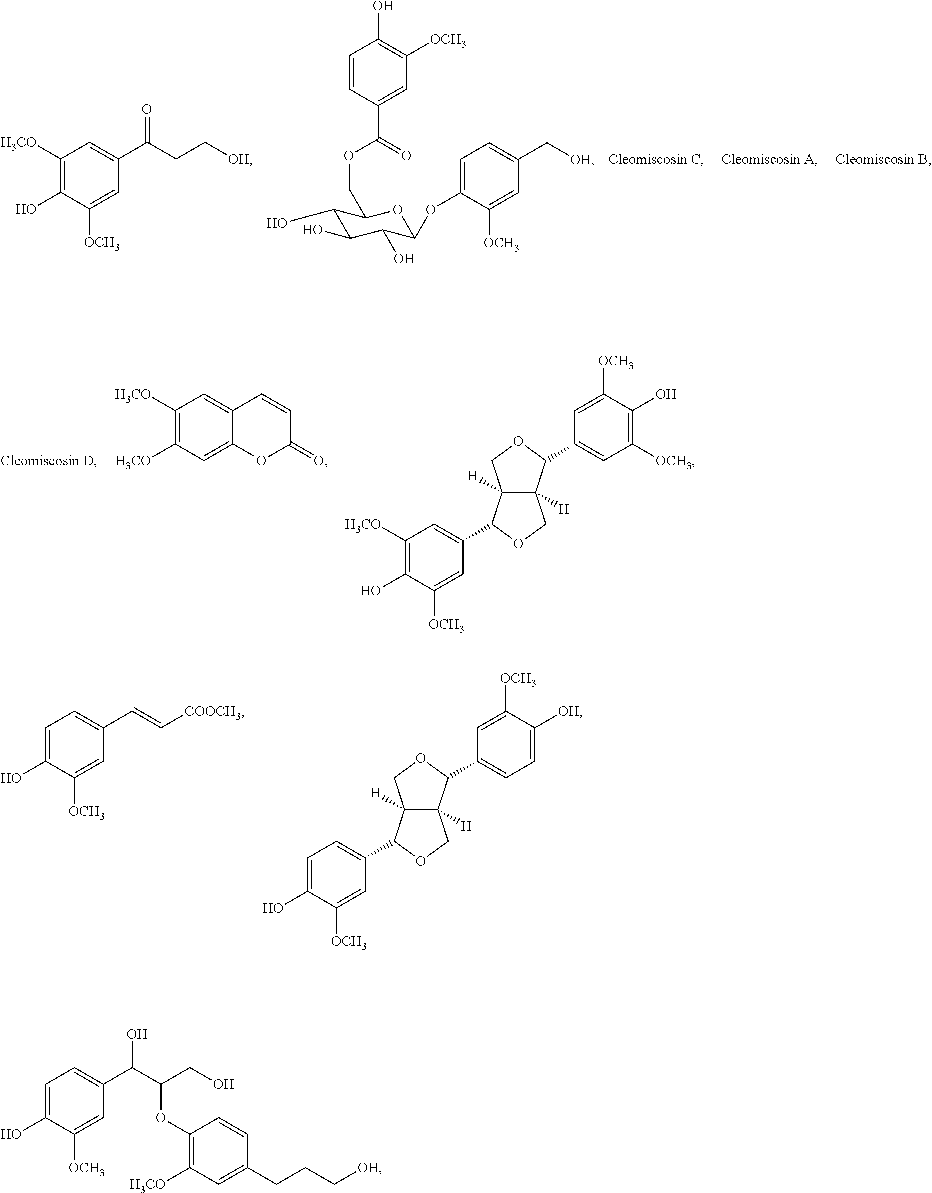

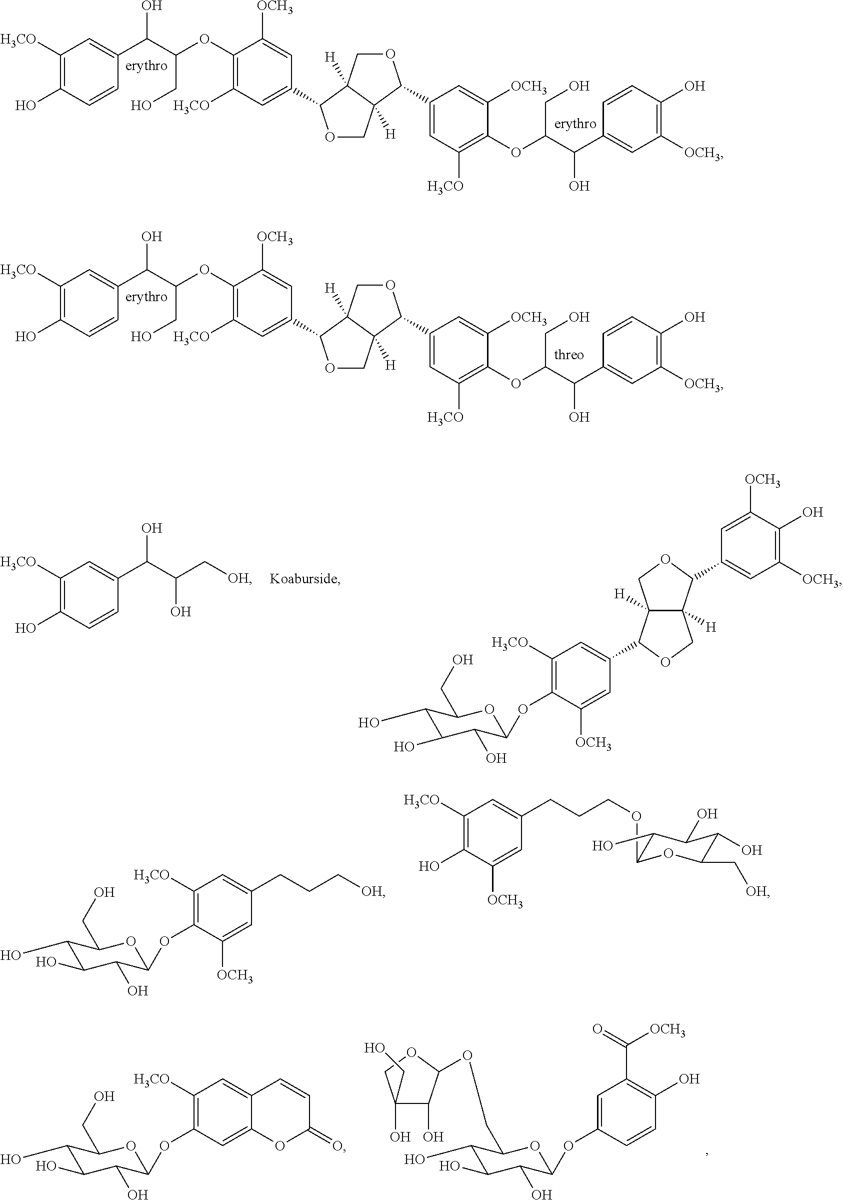

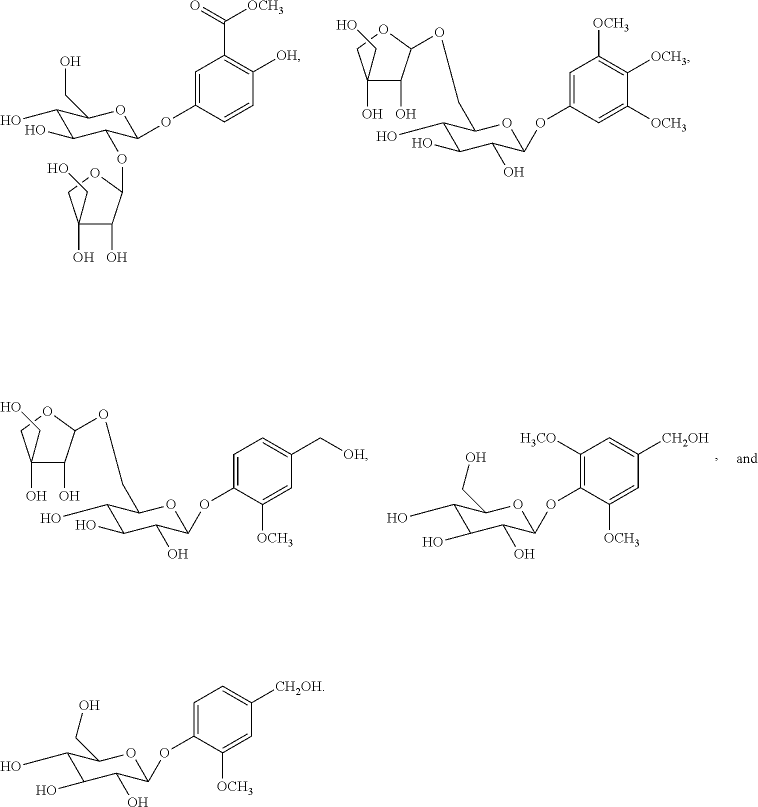

The residue of syrup or syrup-derived product production may comprise diatomaceous earth, celite, kieselguhr, silica, silicon dioxide, calcium, natural sugar sand, ground bones, slop, clay and the like.

The composition may be chosen from a nutraceutical composition, a cosmeceutical composition, a pharmaceutical composition and a functional food composition.

According to an embodiment, there is provided a method of prophylaxis and/or treatment of an ailment comprising administering to a subject in need thereof a composition according to the present invention.

The ailment may be a diabetes, a cancer, an arthritis, a micro-organism infection, a neurodegenerative disease, an inflammatory disease, an oxidative stress related disease, a heart disease, Alzheimer's diseases, a liver disorder a metabolic syndrome, a damaged hepatic function, a hepatic and liver dyslipidemia, a hepatitis, a liver cancer, an atherosclerosis, a hypertension, a skin disease, an eczema, and a psoriasis.

According to an embodiment, there is provided a use of a composition according to the present invention for the prophylaxis and/or treatment of an ailment.

The ailment may be a diabetes, a cancer, an arthritis, a micro-organism infection, a neurodegenerative disease, an inflammatory disease, an oxidative stress related disease, a heart disease, Alzheimer's diseases, a liver disorder a metabolic syndrome, a damaged hepatic function, a hepatic and liver dyslipidemia, a hepatitis, a liver cancer, an atherosclerosis, a hypertension, a skin disease, an eczema, and a psoriasis.

According to an embodiment, there is provided an ingredient composition comprising an extract of an Acer tree in association with an acceptable carrier.

The extract of Acer tree may be an extract from a non-concentrated or concentrated sap, a samara fruit, a samara seed, a stems of leaf, a stem of a samara, a twig, a root, a leaf, a bark, a heartwood, a sapwood, a whole branch, a bark of a branch, a wood of a branch, a sugar a syrup, a syrup extract, a syrup-derived product, a rejection of syrup or syrup-derived product production, a residue of syrup or syrup-derived product production, or combinations thereof.

The syrup derived product may comprise butter, granulated sugar, hardened sugar, soft sugar, taffy, flakes, an extract from lyophilisation of a sap, a maple concentrate or a maple syrup, an extract from drying of a sap, a maple concentrate or a maple syrup, an extract from crystallization of a sap, a maple concentrate or a maple syrup, an extract from pulverization of a sap, a maple concentrate or a maple syrup, an extract from atomization of a sap, a maple concentrate or a maple syrup, an extract from centrigugation of a sap, a maple concentrate or a maple syrup or combinations thereof.

The syrup extract may be chosen from a methanol extract, a butanol extract, a butanol extract with sugar, a butanol extract without sugar, an ethyl acetate extract, an ethanol extract, a 95% ethanol/5% hot water extract, or combinations thereof.

The extract of Acer tree may comprise an extract from concentrated Acer tree water issued from reverse osmosis, a concentrated Acer tree water issued from pre-boiling nanofiltration, a pasteurized Acer tree water, a sterilized Acer tree water, an Acer tree water issued from a high pressure processing, an Acer tree water sterilized by UV irradiation, an Acer tree water sterilized by microwave irradiation and combinations thereof.

The extract of Acer tree may comprise an Acer tree molecule.

The Acer tree molecule may comprise:
Lyoniresinol,
Isolariciresinol,
secoisolariciresinol,
Dehydroconiferyl alcohol,
5′-methoxy-dehydroconiferyl alcohol,
erythro-guaiacylglycerol-β-O-4′-coniferyl alcohol,
erythro-guaiacylglycerol-β-O-4′-dihydroconiferyl alcohol,
[3-[4-[(6-deoxy-α-L-mannopyranosyl)oxy]-3-methoxyphenyl]methyl]-5-(3,4-dimethoxyphenyl)dihydro-3-hydroxy-4-(hydroxymethyl)-2(3H)-furanone,
5-(3″,4″-dimethoxyphenyl)-3-hydroxy-3-(4′-hydroxy-3′-methoxybenzyl)-4-hydroxymethyl-dihydrofuran-2-one,
Scopoletin,
Fraxetin,
Isofraxidin,
Gallic acid,
Ginnalin A (acertannin),
Syringic acid,
Ginnalin B,
Ginnalin C,
Trimethyl gallic acid methyl ester
(E)-3,3′-dimethoxy-4,4′-dihydroxy stilbene,
p-coumaric acid,
Ferulic acid,
(E)-Coniferol,
Syringenin,
Dihydroconiferyl alcohol,
C-veratroylglycol,
2,3-dihydroxy-1-(3,4-dihydroxyphenyl)-1-propanone
2,3-Dihydroxy-1-(4-hydroxy-3,5-dimethoxyphenyl)-1-propanone,
3-Hydroxy-1-(4-hydroxy-3,5-dimethoxyphenyl)propan-1-one,
3′,4′,5′-Trihydroxyacetophenone,
4-Acetylcatechol,
2,4,5-Trihydroxyacetophenone,
1-(2,3,4-trihydroxy-5-methylphenyl)-ethanone,
2-Hydroxy-3′,4′-dihydroxyacetophenone,
Vanillin,
Syringaldehyde,
Catechaldehyde,
3,4-Dihydroxy-2-methylbenzaldehyde,
Catechol,
Catechin,
Epicatechin,
Quebecol,
(erythro, erythro)-1-[4-[2-hydroxy-2-(4-hydroxy-3-methoxyphenyl)-1-(hydroxymethyl)ethoxy]-3,5-dimethoxyphenyl]-1,2,3-propanetriol,
(erythro, threo)-1-[4-[2-hydroxy-2-(4-hydroxy-3-methoxyphenyl)-1-(hydroxymethyl)ethoxy]-3,5-dimethoxyphenyl]-1,2,3-propanetriol,
(threo, erythro)-1-[4-[(2-hydroxy-2-(4-hydroxy-3-methoxyphenyl)-1-(hydroxymethyl)ethoxy]-3-methoxyphenyl]-1,2,3-propanetriol,
(threo, threo)-1-[4-[(2-hydroxy-2-(4-hydroxy-3-methoxyphenyl)-1-(hydroxymethyl)ethoxy]-3-methoxyphenyl]-1,2,3-propanetriol,
threo-guaiacylglycerol-β-O-4′-dihydroconiferyl alcohol,
erythro-1-(4-hydroxy-3-methoxyphenyl)-2-[4-(3-hydroxypropyl)-2,6-dimethoxyphenoxy]-1,3-propanediol,
2-[4-[2,3-dihydro-3-(hydroxymethyl)-5-(3-hydroxypropyl)-7-methoxy-2-benzofuranyl]-2,6-dimethoxyphenoxy]-1-(4-hydroxy-3-methoxyphenyl)-1,3-propanediol,
Acerkinol,
Leptolepisol D,
Buddlenol E,
(1S,2R)-2-[2,6-dimethoxy-4-[(1S,3aR,4S,6aR)-tetrahydro-4-(4-hydroxy-3,5-dimethoxyphenyl)-1H,3H-furo[3,4-c]furan-1-yl]phenoxy]-1-(4-hydroxy-3-methoxyphenyl)-1,3-propanediol,
Syringaresinol,
Icariside E4,
Sakuraresinol,
1,2-diguaiacyl-1,3-propanediol
protocatechuic acid,
4-(dimethoxymethyl)-pyrocatechol,
Tyrosol,
4-hydroxycatechol,
Phaseic acid,
Nortrachelogenin 8′-O-β-D-glucopyranoside,
3-O-galloyl-1,5-anhydro-D-glucitol,
4-O-galloyl-1,5-anhydro-D-glucitol,
2,4-di-O-galloyl-1,5-anhydro-D-glucitol,
Quercetin-3-O-α-rhamnopyranoside,
2,3-di-O-galloyl-1,5-anhydro-D-glucitol,
3-Methoxy-4-hydroxyphenol 1-O-β-D-(6′-O-galloyl)-glucopyranoside,
3,5-Dihydroxy-4-methoxybenzoic acid methyl ester,
7,8-Dihydroxy-6-methoxycoumarin,
4-Methoxy-3,5-dihydroxybenzoic acid,
Methyl 3,5-dimethoxy-4-hydroxybenzoate,
Methyl vanillate,
Methyl gallate,
3,6-di-O-galloyl-1,5-anhydro-D-glucitol,
(+)-Epicatechin-(4β-8,2β-O-7)-catechin,
Epicatechin gallate,
(+)-Epicatechin-(4β-8,2β-O-7)-epicatechin,
Quercetin-3-O-(3″-O-galloyl)-α-rhamnopyranoside,
Quercetin-3-O-(2″-O-galloyl)-α-rhamnopyranoside,
2,4,64′-O-galloyl-1,5-anhydro-D-glucitol,

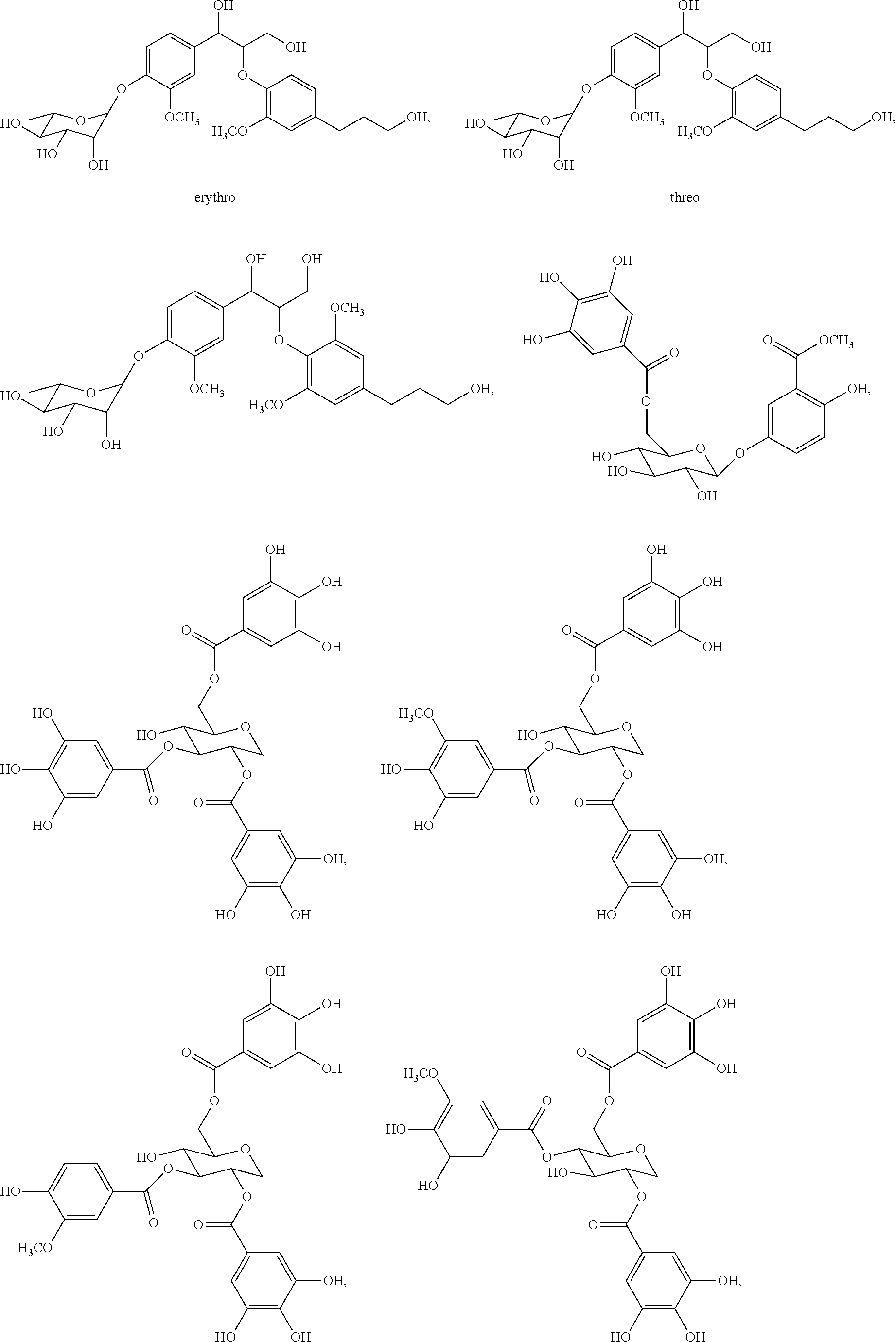

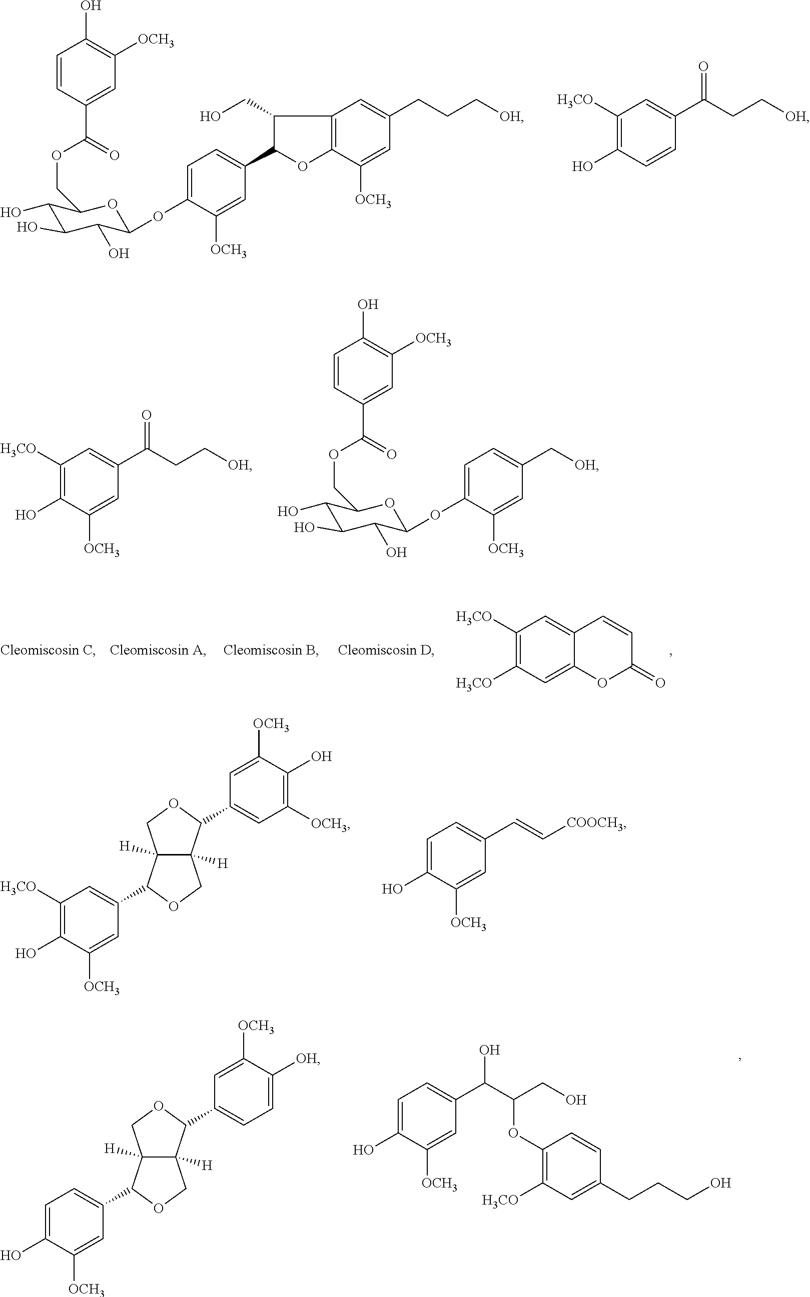

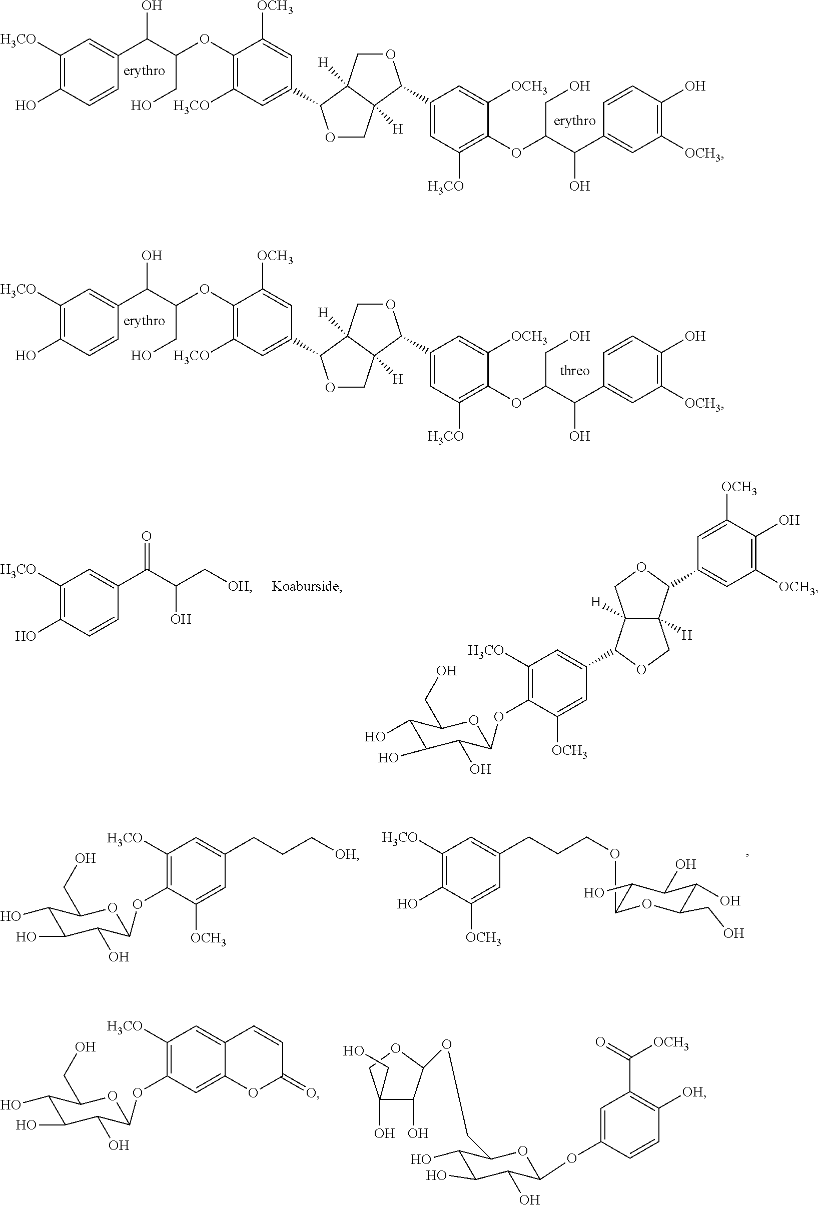

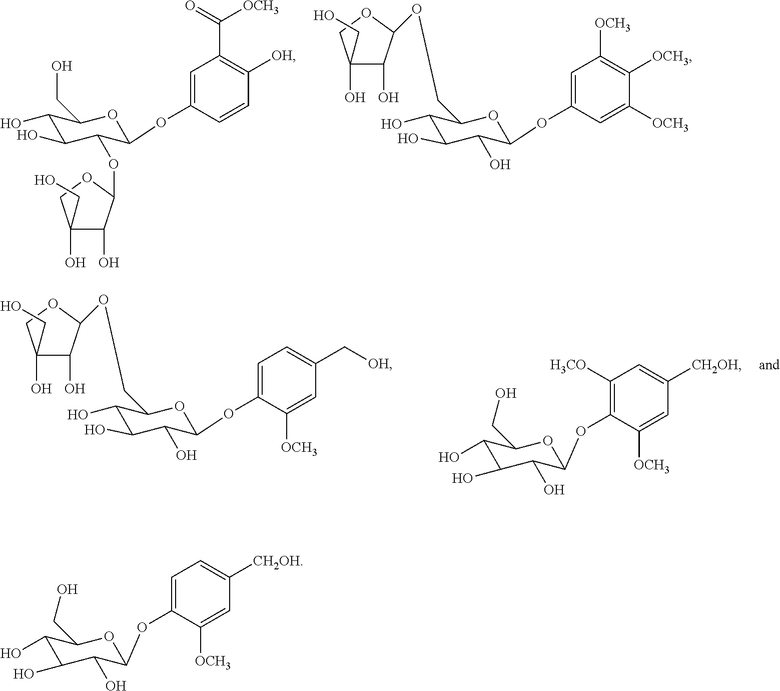

The residue of syrup or syrup-derived product production may comprise diatomaceous earth, celite, kieselguhr, silica, silicon dioxide, calcium, natural sugar sand, ground bones, slop, clay and the like.

The Acer tree may be chosen from Acer nigrum, Acer lanum, Acer acuminatum, Acer albopurpurascens, Acer argutum, Acer barbinerve, Acer buergerianum, Acer caesium, Acer campbellii, Acer campestre, Acer capillipes, Acer cappadocicum, Acer carpinifolium, Acer caudatifolium, Acer caudatum, Acer cinnamomifolium, Acer circinatum, Acer cissifolium, Acer crassum, Acer crataegifolium, Acer davidii, Acer decandrum, Acer diabolicum, Acer distylum, Acer divergens, Acer erianthum, Acer erythranthum, Acer fabri, Acer garrettii, Acer glabrum, Acer grandidentatum, Acer griseum, Acer heldreichii, Acer henryi, Acer hyrcanum, Acer ibericum, Acer japonicum, Acer kungshanense, Acer kweilinense, Acer laevigatum, Acer laurinum, Acer lobelii, Acer lucidum, Acer macrophyllum, Acer mandshuricum, Acer maximowiczianum, Acer miaoshanicum, Acer micranthum, Acer miyabei, Acer mono, Acer mono×Acer truncatum, Acer monspessulanum, Acer negundo, Acer ningpoense, Acer nipponicum, Acer oblongum, Acer obtusifolium, Acer oliverianum, Acer opalus, Acer palmatum, Acer paxii, Acer pectinatum, Acer pensylvanicum, Acer pentaphyllum, Acer pentapomicum, Acer pictum, Acer pilosum, Acer platanoides, Acer poliophyllum, Acer pseudoplatanus, Acer pseudosieboldianum, Acer pubinerve, Acer pycnanthum, Acer rubrum, Acer rufinerve, Acer saccharinum, Acer saccharum, Acer sempervirens, Acer shirasawanum, Acer sieboldianum, Acer sinopurpurescens, Acer spicatum, Acer stachyophyllum, Acer sterculiaceum, Acer takesimense, Acer tataricum, Acer tegmentosum, Acer tenuifolium, Acer tetramerum, Acer trautvetteri, Acer triflorum, Acer truncatum, Acer tschonoskii, Acer turcomanicum, Acer ukurunduense, Acer velutinum, Acer wardii, Acer x peronai, and Acer x pseudoheldreichii.

The Acer tree may be chosen from Acer Saccharum and Acer Rubrum L.

The ingredient composition may be a food ingredient composition, a non-food ingredient composition, or combination thereof.

According to an embodiment, there is provided a method of seasoning a food comprising administering to a food an ingredient composition according to the present invention.

According to an embodiment, there is provided an Acer tree essential oil composition comprising

a hydrophobic fraction extracted from an Acer tree biomass; and

a suitable solvent.

The Acer tree biomass may be from at least one of a samara fruit, a samara seed, a stem of leaf, a stem of a samara, a twig, a root, a leaf, a bark, a heartwood, a sapwood, a whole branch, a bark of a branch, a wood of a branch.

The hydrophobic fraction extracted from an Acer tree biomass may comprise an Acer tree molecule.

The Acer tree molecule may comprise:
Lyoniresinol,
Isolariciresinol,
secoisolariciresinol,
Dehydroconiferyl alcohol,
5′-methoxy-dehydroconiferyl alcohol,
erythro-guaiacylglycerol-O-β-4′-coniferyl alcohol,
erythro-guaiacylglycerol-β-O-4′-dihydroconiferyl alcohol,
[3-[4-[(6-deoxy-α-L-mannopyranosyl)oxy]-3-methoxyphenyl]methyl]-5-(3,4-dimethoxyphenyl)dihydro-3-hydroxy-4-(hydroxymethyl)-2(3H)-furanone,
5-(3″,4″-dimethoxyphenyl)-3-hydroxy-3-(4′-hydroxy-3′-methoxybenzyl)-4-hydroxymethyl-dihydrofuran-2-one,
Scopoletin,
Fraxetin,
Isofraxidin,
Gallic acid,
Ginnalin A (acertannin),
Syringic acid,
Ginnalin B,
Ginnalin C,
Trimethyl gallic acid methyl ester
(E)-3,3′-dimethoxy-4,4′-dihydroxy stilbene,
p-coumaric acid,
Ferulic acid,
(E)-Coniferol,
Syringenin,
Dihydroconiferyl alcohol,
C-veratroylglycol,
2,3-dihydroxy-1-(3,4-dihydroxyphenyl)-1-propanone
2,3-Dihydroxy-1-(4-hydroxy-3,5-dimethoxyphenyl)-1-propanone,
3-Hydroxy-1-(4-hydroxy-3,5-dimethoxyphenyl)propan-1-one,
3′,4′,5′-Trihydroxyacetophenone,
4-Acetylcatechol,
2,4,5-Trihydroxyacetophenone,
1-(2,3,4-trihydroxy-5-methylphenyl)-ethanone,
2-Hydroxy-3′,4′-dihydroxyacetophenone,
Vanillin,
Syringaldehyde,
Catechaldehyde,
3,4-Dihydroxy-2-methylbenzaldehyde,
Catechol,
Catechin,
Epicatechin,
Quebecol,
(erythro, erythro)-1-[4-[2-hydroxy-2-(4-hydroxy-3-methoxyphenyl)-1-(hydroxymethyl)ethoxy]-3,5-dimethoxyphenyl]-1,2,3-propanetriol,
(erythro, threo)-1-[4-[2-hydroxy-2-(4-hydroxy-3-methoxyphenyl)-1-(hydroxymethyl)ethoxy]-3,5-dimethoxyphenyl]-1,2,3-propanetriol,
(threo, erythro)-1-[4-[(2-hydroxy-2-(4-hydroxy-3-methoxyphenyl)-1-(hydroxymethyl)ethoxy]-3-methoxyphenyl]-1,2,3-propanetriol,
(threo, threo)-1-[4-[(2-hydroxy-2-(4-hydroxy-3-methoxyphenyl)-1-(hydroxymethyl)ethoxy]-3-methoxyphenyl]-1,2,3-propanetriol,
threo-guaiacylglycerol-β-O-4′-dihydroconiferyl alcohol,
erythro-1-(4-hydroxy-3-methoxyphenyl)-2-[4-(3-hydroxypropyl)-2,6-dimethoxyphenoxy]-1,3-propanediol,
2-[4-[2,3-dihydro-3-(hydroxymethyl)-5-(3-hydroxypropyl)-7-methoxy-2-benzofuranyl]-2,6-dimethoxyphenoxy]-1-(4-hydroxy-3-methoxyphenyl)-1,3-propanediol,
Acerkinol,
Leptolepisol D,
Buddlenol E,
(1S,2R)-2-[2,6-dimethoxy-4-[(1S,3aR,4S,6aR)-tetrahydro-4-(4-hydroxy-3,5-dimethoxyphenyl)-1H,3H-furo[3,4-c]furan-1-yl]phenoxy]-1-(4-hydroxy-3-methoxyphenyl)-1,3-propanediol,
Syringaresinol,
Icariside E4,
Sakuraresinol,
1,2-diguaiacyl-1,3-propanediol
protocatechuic acid,
4-(dimethoxymethyl)-pyrocatechol,
Tyrosol,
4-hydroxycatechol,
Phaseic acid,
Nortrachelogenin 8′-O-β-D-glucopyranoside,
3-O-galloyl-1,5-anhydro-D-glucitol,
4-O-galloyl-1,5-anhydro-D-glucitol,
2,4-di-O-galloyl-1,5-anhydro-D-glucitol,
Quercetin-3-O-α-rhamnopyranoside,
2,3-di-O-galloyl-1,5-anhydro-D-glucitol,
3-Methoxy-4-hydroxyphenol 1-O-β-D-(6′-O-galloyl)-glucopyranoside,
3,5-Dihydroxy-4-methoxybenzoic acid methyl ester,
7,8-Dihydroxy-6-methoxycoumarin,
4-Methoxy-3,5-dihydroxybenzoic acid,
Methyl 3,5-dimethoxy-4-hydroxybenzoate,
Methyl vanillate,
Methyl gallate,
3,6-di-O-galloyl-1,5-anhydro-D-glucitol,
(+)-Epicatechin-(4β-8,2β-O-7)-catechin,
Epicatechin gallate,
(+)-Epicatechin-(4β-8,2β-O-7)-epicatechin,
Quercetin-3-O-(3″-O-galloyl)-α-rhamnopyranoside,
Quercetin-3-O-(2″-O-galloyl)-α-rhamnopyranoside,
2,4,6-tri-O-galloyl-1,5-anhydro-D-glucitol,

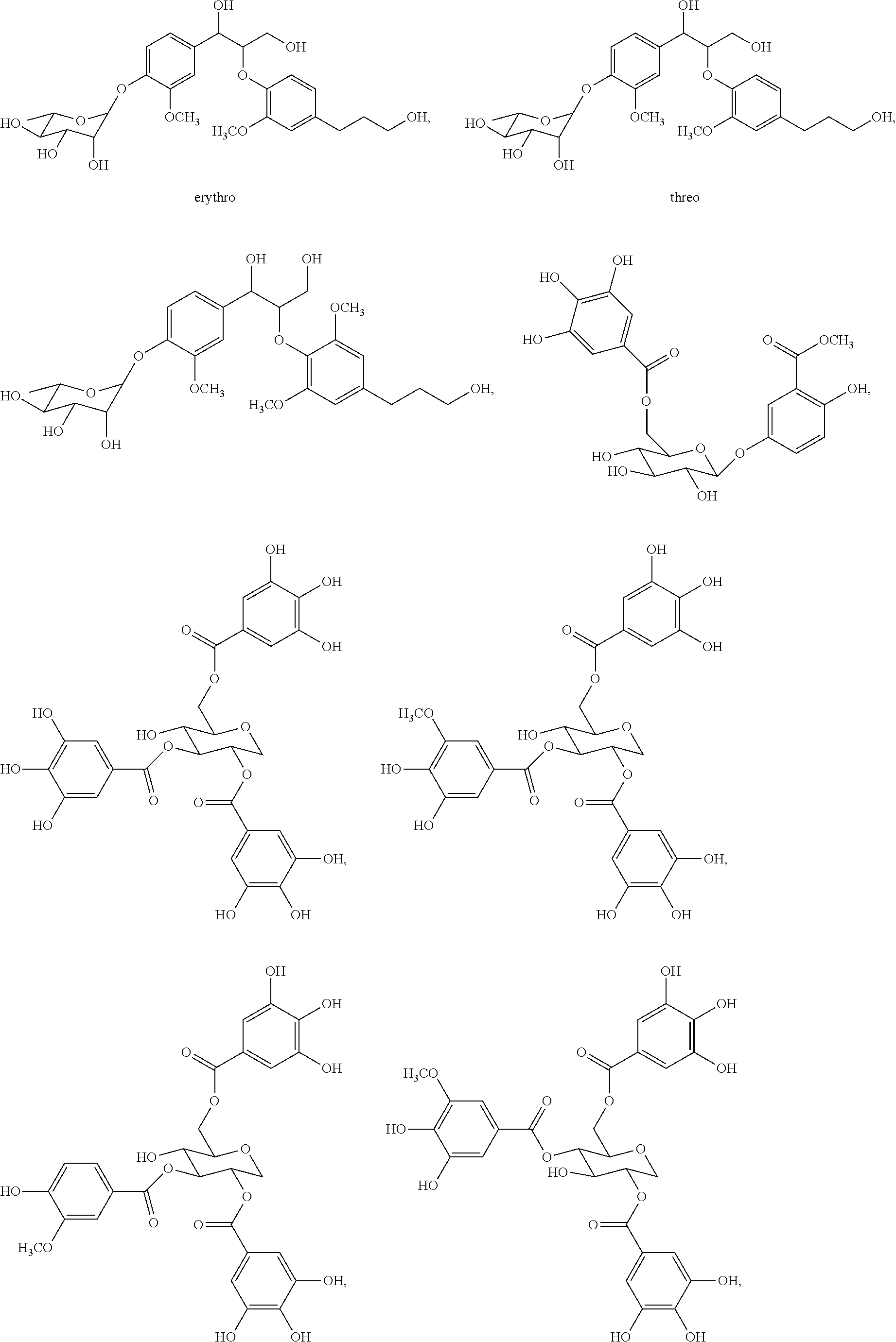

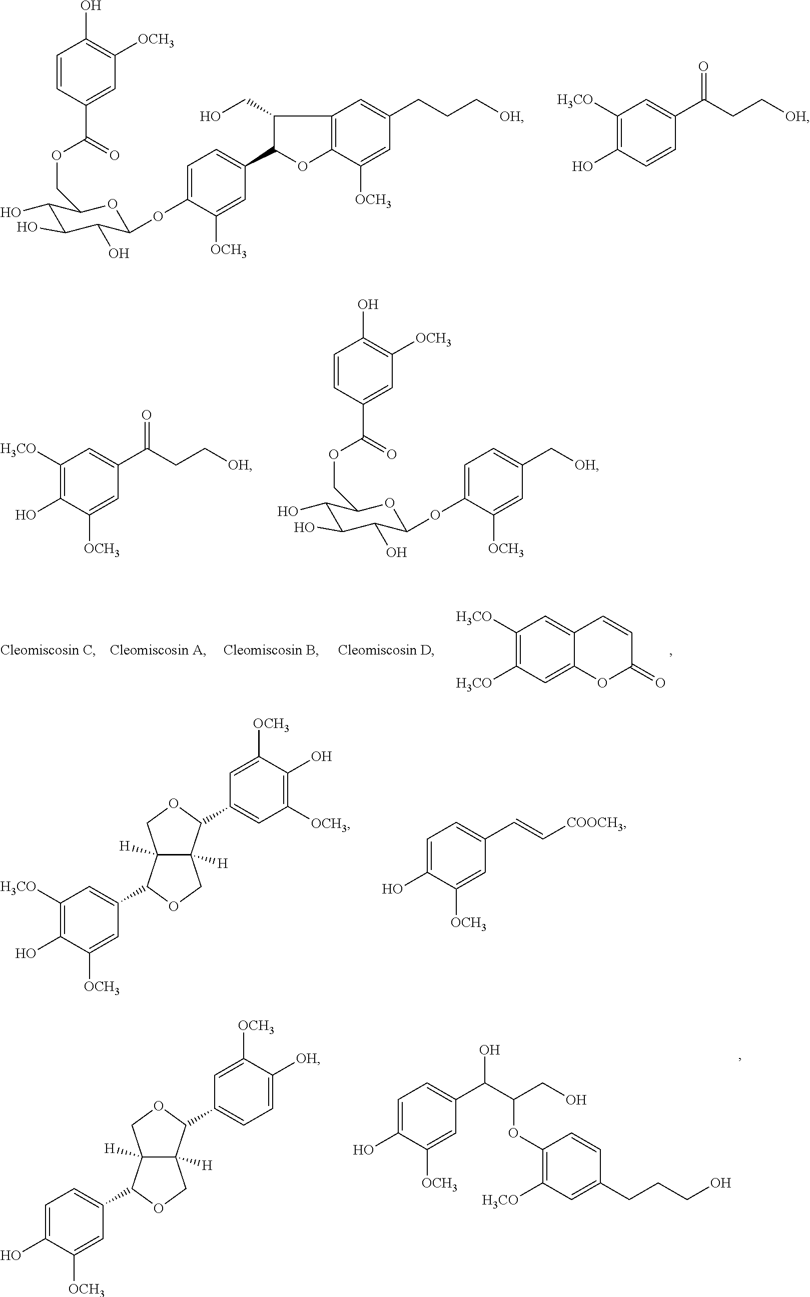

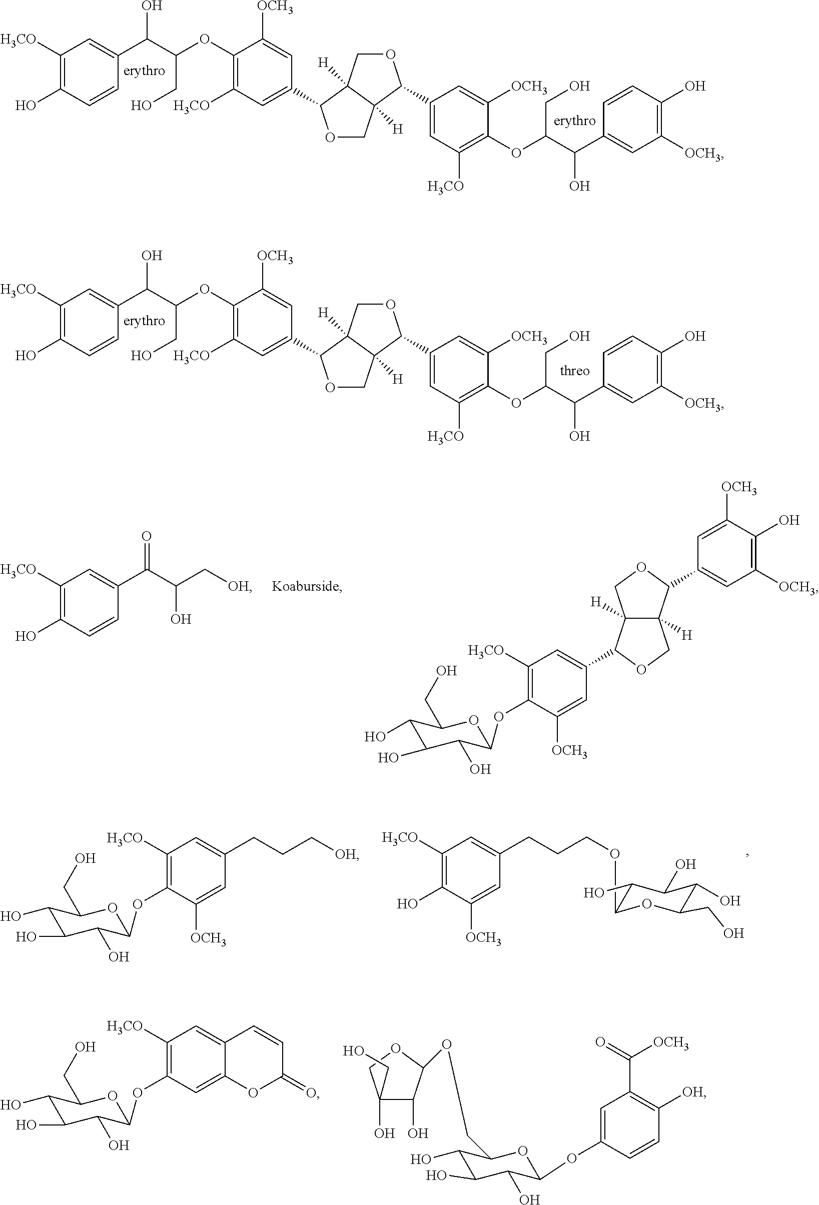

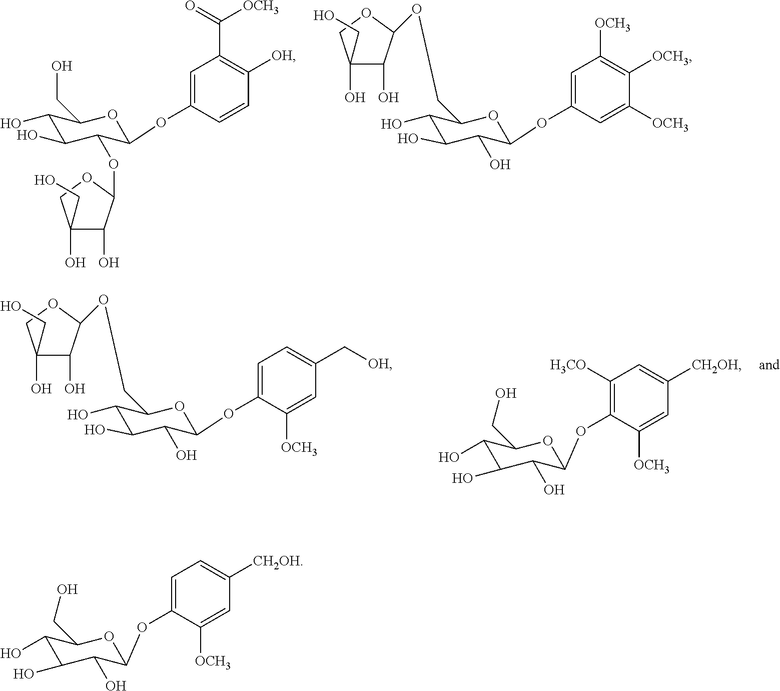

The suitable solvent may be at least one of ethanol, polyethylene glycol, or a pharmaceutically acceptable carrier oil.

The pharmaceutically acceptable carrier oil may be at least one of sweet almond oil, kukui nut oil, apricot kernel oil, macadamia nut oil, avocado oil, meadowfoam oil, borage seed oil, olive oil, camellia seed oil, peanut oil, cranberry seed oil, pecan oil, evening primrose oil, pomegranate seed oil, fractionated coconut oil, rose hip oil, grapeseed oil, seabuckthorn berry oil, hazelnut oil, sesame oil, hemp seed oil, sunflower oil, jojoba, and watermelon seed oil.

According to an embodiement, there is provided a method of preparing a maple syrup comprising adding a hydrophobic fraction extracted from an Acer tree biomass before or during the preparation of a maple syrup.

According to an embodiement, there is provided a food composition comprising:
a plurality of Acer tree samara.

The Acer tree samara may be germinated.

The Acer tree samara may comprise a fruit.

The Acer tree samara may comprise a seed.

The Acer tree samara may be dried and/or fermented.

The Acer tree samara may be dessicated.

The germinated Acer tree samara may be marinated.

The food composition may further comprise a seasoning ingredient chosen from a salt, a pepper, a cheese, an oil, a vinegar, a salad sauce, and a vinaigrette.

The salt may be chosen from sodium chloride, a sea salt, and sodium acetate.

According to an embodiment, there is provided a solid sweetening composition for oral consumption comprising:

a Acer tree sugar extract;

at least one sweetener, and
a dietary acceptable filler.

The Acer tree sugar extract may be at least one of non-concentrated or concentrated sap, syrup, a syrup, a syrup extract, a syrup-derived product, a rejection of syrup or syrup-derived product production, a residue of syrup or syrup-derived product production, taffy, flakes, sugar, spread, an extract from lyophilisation of a sap, a maple concentrate or a maple syrup, an extract from drying of a sap, a maple concentrate or a maple syrup, an extract from crystallization of a sap, a maple concentrate or a maple syrup, an extract from pulverization of a sap, a maple concentrate or a maple syrup, an extract from atomization of a sap, a maple concentrate or a maple syrup, an extract from centrifugation of a sap, a maple concentrate or a maple syrup or combinations thereof.

The syrup derived products may comprise butter, granulated sugar, hardened sugar, soft sugar, taffy, flakes, or combinations thereof.

The syrup extracts may be chosen from a methanol extract, a butanol extract, a butanol extract with sugar, a butanol extract without sugar, an ethyl acetate extract, an ethanol extract, a 95% ethanol/5% hot water extract, or combinations thereof.

The Acer tree sugar extract may comprise an Acer tree molecule.

The Acer tree molecule may comprise:
Lyoniresinol,
Isolariciresinol,
secoisolariciresinol,
Dehydroconiferyl alcohol,
5′-methoxy-dehydroconiferyl alcohol,
erythro-guaiacylglycerol-β-O-4′-coniferyl alcohol,
erythro-gualacylglycerol-β-O-4′-dihydroconiferyl alcohol,
[3-[4-[(6-deoxy-α-L-mannopyranosyl)oxy]-3-methoxyphenyl]methyl]-5-(3,4-dimethoxyphenyl)dihydro-3-hydroxy-4-(hydroxymethyl)-2(3H)-furanone,
5-(3″,4″-dimethoxyphenyl)-3-hydroxy-3-(4′-hydroxy-3′-methoxybenzyl)-4-hydroxymethyl-dihydrofuran-2-one,
Scopoletin,
Fraxetin,
Isofraxidin,
Gallic acid,
Ginnalin A (acertannin),
Syringic acid,
Ginnalin B,
Ginnalin C,
Trimethyl gallic acid methyl ester
(E)-3,3′-dimethoxy-4,4′-dihydroxy stilbene,
p-coumaric acid,
Ferulic acid,
(E)-Coniferol,
Syringenin,
Dihydroconiferyl alcohol,
C-veratroylglycol,
2,3-dihydroxy-1-(3,4-dihydroxyphenyl)-1-propanone
2,3-Dihydroxy-1-(4-hydroxy-3,5-dimethoxyphenyl)-1-propanone,
3-Hydroxy-1-(4-hydroxy-3,5-dimethoxyphenyl)propan-1-one,
3′,4′,5′-Trihydroxyacetophenone,
4-Acetylcatechol,
2,4,5-Trihydroxyacetophenone,
1-(2,3,4-trihydroxy-5-methylphenyl)-ethanone,
2-Hydroxy-3′,4′-dihydroxyacetophenone,
Vanillin,
Syringaldehyde,
Catechaldehyde,
3,4-Dihydroxy-2-methylbenzaldehyde,
Catechol,
Catechin,
Epicatechin,
Quebecol,
(erythro, erythro)-1-[4-[2-hydroxy-2-(4-hydroxy-3-methoxyphenyl)-1-(hydroxymethyl)ethoxy]-3,5-dimethoxyphenyl]-1,2,3-propanetriol,
(erythro, threo)-1-[4-[2-hydroxy-2-(4-hydroxy-3-methoxyphenyl)-1-(hydroxymethyl)ethoxy]-3,5-dimethoxyphenyl]-1,2,3-propanetriol,
(threo, erythro)-1-[4-[(2-hydroxy-2-(4-hydroxy-3-methoxyphenyl)-1-(hydroxymethyl)ethoxy]-3-methoxyphenyl]-1,2,3-propanetriol,
(threo, threo)-1-[44(2-hydroxy-2-(4-hydroxy-3-methoxyphenyl)-1-(hydroxymethyl)ethoxy]-3-methoxyphenyl]-1,2,3-propanetriol,
threo-guaiacylglycerol-(3-O-4′-dihydroconiferyl alcohol,
erythro-1-(4-hydroxy-3-methoxyphenyl)-2-[4-(3-hydroxypropyl)-2,6-dimethoxyphenoxy]-1,3-propanediol,
2-[4-[2,3-dihydro-3-(hydroxymethyl)-5-(3-hydroxypropyl)-7-methoxy-2-benzofuranyl]-2,6-dimethoxyphenoxy]-1-(4-hydroxy-3-methoxyphenyl)-1,3-propanediol,
Acerkinol,
Leptolepisol D,
Buddlenol E,
(1S,2R)-2-[2,6-dimethoxy-4-[(1S,3aR,4S,6aR)-tetrahydro-4-(4-hydroxy-3,5-dimethoxyphenyl)-1H,3H-furo[3,4-c]furan-1-yl]phenoxy]-1-(4-hydroxy-3-methoxyphenyl)-1,3-propanediol,
Syringaresinol,
Icariside E4,
Sakuraresinol,
1,2-diguaiacyl-1,3-propanediol
protocatechuic acid,
4-(dimethoxymethyl)-pyrocatechol,
Tyrosol,
4-hydroxycatechol,
Phaseic acid,
Nortrachelogenin 8′-O-β-D-glucopyranoside,
3-O-galloyl-1,5-anhydro-D-glucitol,
4-O-galloyl-1,5-anhydro-D-glucitol,
2,4-di-O-galloyl-1,5-anhydro-D-glucitol,
Quercetin-3-O-α-rhamnopyranoside,
2,3-di-O-galloyl-1,5-anhydro-D-glucitol,
3-Methoxy-4-hydroxyphenol 1-O-β-D-(6′-O-galloyl)-glucopyranoside,
3,5-Dihydroxy-4-methoxybenzoic acid methyl ester,
7,8-Dihydroxy-6-methoxycoumarin,
4-Methoxy-3,5-dihydroxybenzoic acid,
Methyl 3,5-dimethoxy-4-hydroxybenzoate,
Methyl vanillate,
Methyl gallate,
3,6-di-O-galloyl-1,5-anhydro-D-glucitol,
(+)-Epicatechin-(4β-8,2β-O-7)-catechin,
Epicatechin gallate,
(+)-Epicatechin-(4β-8,2β-O-7)-epicatechin,
Quercetin-3-O-(3″-O-galloyl)-α-rhamnopyranoside,
Quercetin-3-O-(2″-O-galloyl)-α-rhamnopyranoside,
2,4,6-tri-O-galloyl-1,5-anhydro-D-glucitol,

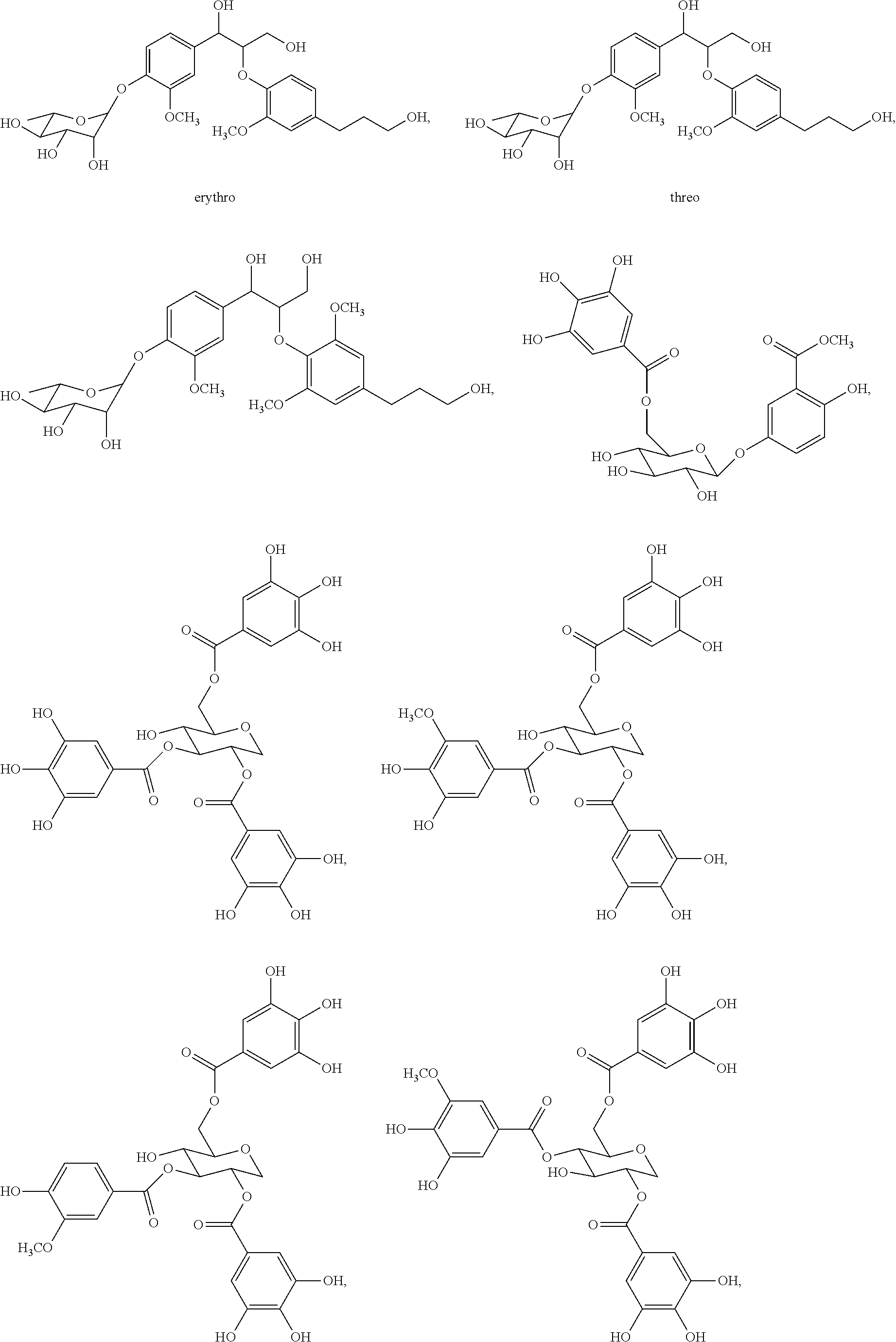

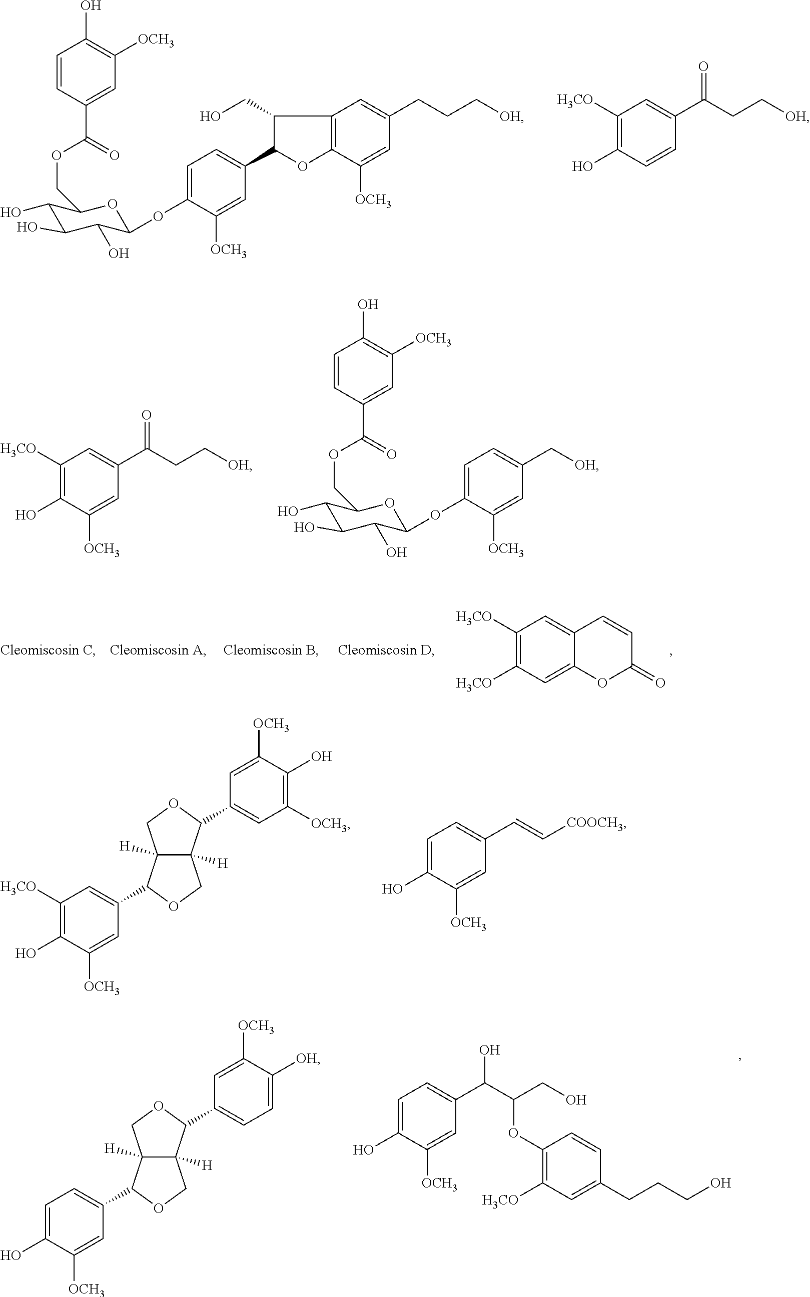

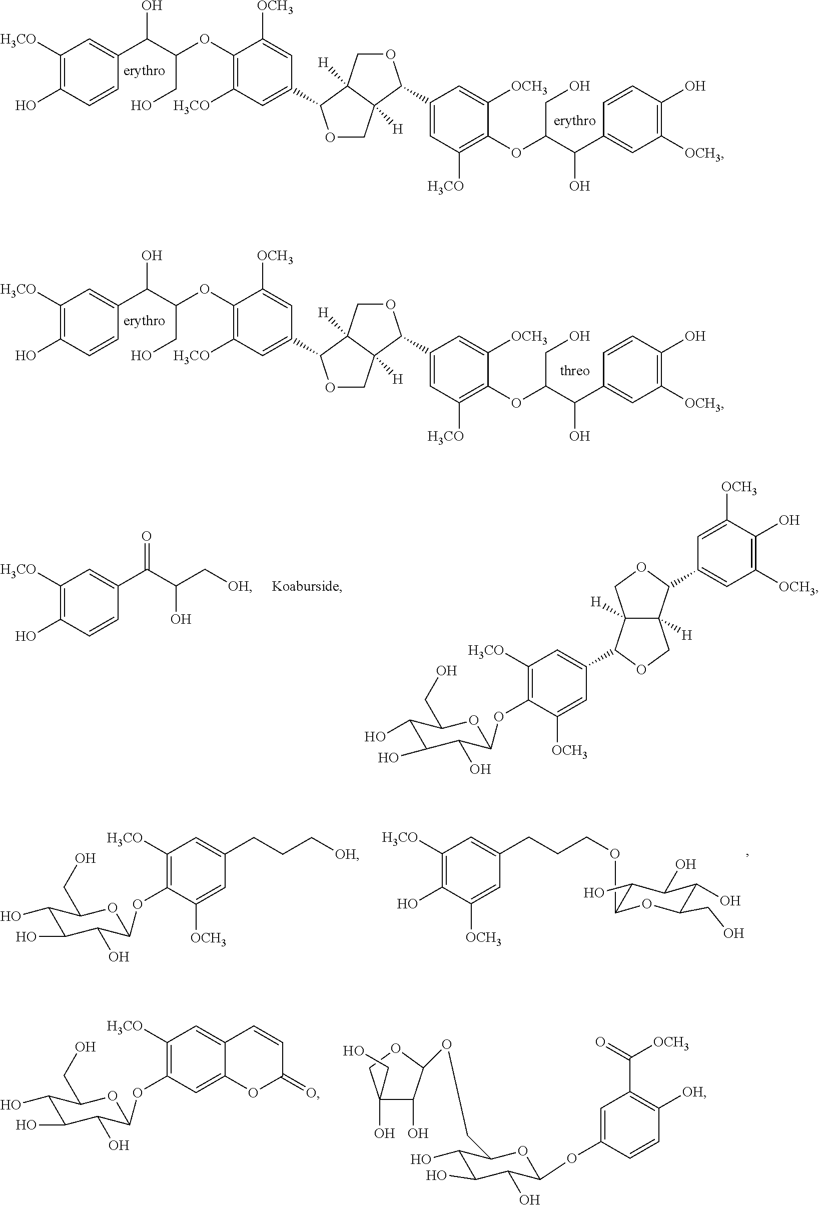

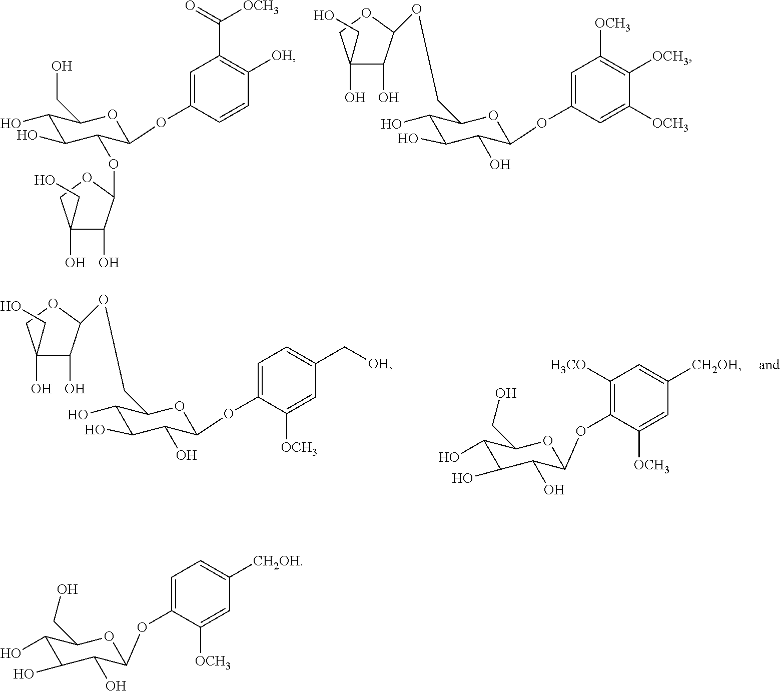

The residue of syrup or syrup-derived product production may comprise diatomaceous earth, celite, kieselguhr, silica, silicon dioxide, calcium, natural sugar sand, ground bones, slop, clay and the like.

The at least one sweetener may be chosen from a nutritive sweetener and a non-nutritive sweetener.

The nutritive sweetener may be at least one of honey, birch syrup, pine syrup, hickory syrup, poplar syrup, palm syrup, sugar beet syrup, sorghum syrup, corn syrup, cane syrup, golden syrup, barley malt syrup, a molasse, brown rice syrup, agave nectar, yacon syrup, fructose, maltitol, brown sugar, Okinawa syrup or combinations thereof.

The non-nutritive sweetener may be at least one of adenosine monophosphate, acesulfame potassium, alitame, aspartame, anethole, cyclamate, glycyrrhizin, lo han guo, miraculin, neotame, perillartine, saccharin, selligueain A, a Stevia rebaudiana extract, sucralose, thaumatin, neohesperdine DC, thavmatin, brazzein and inulin.

The Stevia rebaudiana extract may comprise at least one of stevioside, rebaudioside A, rebaudioside B, and rebaudioside C.

According to an embodiement, there is provided an infusion composition for the preparation of a beverage comprising:
an extract of an Acer tree leaf, samara fruit, samara seed, root, samara stem leaf stem, twig, heartwood, sapwood, a whole branch, a bark of a branch, a wood of a branch, and/or bark.

The infusion composition may further comprise a herbal component.

The herbal component may be at least one of a tea, and a herbal tea.

The tea may be at least one of Bai Hao Yinzhen tea, Bai Mu Dan tea, Pai Mu Tan tea, Gong Mei tea, Shou Mei tea, White Puerh tea, Ceylon White tea, Darjeeling White tea, Assam White tea, African White tea, Junshan Yinzhen tea, Huoshan Huangya tea, Meng Ding tea, Huangya tea, Da Ye Qing tea, Huang Tang tea, Junshan Yinzhen tea, Longjing tea, Hui Ming tea, Long Ding tea, Hua Ding tea, Qing Ding tea, Gunpowder tea, Bi Luo Chun tea, Rain Flower tea, Shui Xi Cui Bo tea, Camellia Sinensis tea, Yu Lu tea, Xin Yang Mao Jian tea, Chun Mee tea, Gou Gu Nao tea, Yun Wu tea, Da Fang tea, Huangshan Maofeng tea, Lu'An Guapian tea, Hou Kui tea, Tun Lu tea, Huo Qing tea, Wuliqing tea, Hyson tea, Zhu Ye Qing tea, Meng Ding Can Lu tea, Genmaicha tea, Gyokuro tea, Kabusecha tea, Sencha tea, Fukamushicha tea, Tamaryokucha tea, Bancha tea, Kamairicha tea, Kukicha tea, Mecha tea, Konacha tea, Matcha tea, Genmaicha tea, Bancha tea, Höjicha tea, Tencha tea, Aracha tea, Shincha tea, funmatsucha tea or combinations thereof.

The herbal tea may be at least one of anise tea, artichoke tea, roasted barley tea, bee balm tea, boldo tea, cannabis tea, catnip tea, Hex causue leaves tea, cinnamon tea, coffee leaves tea, coffee cherry tea, Cerasse tea, dried chamomile blossoms tea, chrysanthemum tea, citrus peel tea, bergamot tea, orange peel tea, dandelion tea, dill tea, echinacea tea, essiac tea, fennel tea, gentian tea, ginger root tea, ginseng tea, hawthorn tea, hibiscus tea, rose hip tea, honeybush tea, horehound tea, hydrangea tea, Jiaogulan tea, Kapor tea, Kava root tea, Ku Ding tea, Labrador tea, Lapacho tea, lemon balm tea, lemon grass tea, licorice root tea, lime blossom tea, yerba mate tea, mate de coca tea, mint tea, european mistletoe tea, neem leaf tea, nettle leaf tea, asiatic pennywort leaf tea, pennyroyal leaf tea, pine tea, red raspberry leaf tea, scorched rice tea, rooibos tea, roselle petals, rosemary memory herb tea, sage tea, skullcap tea, serendib tea, sobacha, spicebush leaf tea, spruce tea, staghorn sumac fruit tea, stevia tea, St. John's Wort tea, tulsi tea, uncaria tomentosa, valerian tea, Verbena tea, vetiver tea, roasted wheat tea, wax gourd tea, Wong Logat tea, woodruff tea, yarrow tea, yerba mate tea, yuen kut lam kam wo tea or combinations thereof.

According to an embodiment, there is provided a method of infusing an infusion composition according to the present invention, wherein said infusion composition is infused with a maple tree based matrix.

The maple tree based matrix may be chosen from a maple tree sap, a concentrated maple tree water, a maple tree syrup.

According to an embodiement, there is provided a compound of formula (I), or a pharmaceutically acceptable salt thereof:

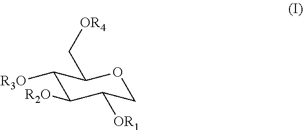

wherein R₁ and R₄ are each independently chosen from
(a) C₁₋₃ alkyl substituted with 0-3 groups selected from halogen, —OH, —OCH₃,
(b) —C(═O)H,
(c) —C(═O)C₁₋₃ alkyl substituted with 0-3 groups selected from halogen, —OH, —OCH₃,
(d) —CN,
(e) —HC—NOH,
(f) —(CH₃)C═NOH,
(g) —HC═NOC₁₋₃ alkyl substituted with 0-3 groups selected from halogen, —OH, —OCH₃,
(h) —(CH₃)C═NOC₁₋₃ alkyl substituted with 0-3 groups selected from halogen, —OH, —OCH₃,
(i) —C(═O)OC₁₋₃ alkyl substituted with 0-3 groups selected from halogen, —OH, —OCH₃,
(j) aryl substituted with 0-5 group selected from halogen, —OH, —OCH₃,
(k) —C(═O) aryl substituted with 0-5 group selected from halogen, —OH, —OCH₃,
(l) HET-aryl wherein HET is a 5 or 6-membered heteroaromatic ring containing 1-3 heteroatoms selected from O, N and S, wherein said HET-aryl is substituted with 0-5 group selected from halogen, —OH, —OCH₃,
(m) —C(═O) HET-aryl wherein HET is a 5 or 6-membered heteroaromatic ring containing 1-3 heteroatoms selected from O, N and S, wherein said HET-aryl is substituted with 0-5 group selected from halogen, —OH, —OCH₃,
(n) cinnamon acyl substituted with 0-5 group selected from halogen, —OH, —OCH₃, and

R₂ and R₃ are each independently chosen from
(o) C₁₋₃ alkyl substituted with 0-3 groups selected from halogen, —OH, —OCH₃,
(p) —C(═O)H,
(q) —C(═O)C₁₋₃ alkyl substituted with 0-3 groups selected from halogen, —OH, —OCH₃,
(r) —CN,
(s) —HC—NOH,
(t) —(CH₃)C═NOH,
(u) —HC═NOC₁₋₃ alkyl substituted with 0-3 groups selected from halogen, —OH, —OCH₃,
(v) —(CH₃)C═NOC₁₋₃ alkyl substituted with 0-3 groups selected from halogen, —OH, —OCH₃,
(w) —C(═O)OC₁₋₃ alkyl substituted with 0-3 groups selected from halogen, —OH, —OCH₃,
(x) aryl substituted with 0-5 group selected from halogen, —OH, —OCH₃,
(y) —C(═O) aryl substituted with 0-5 group selected from halogen, —OH, —OCH₃,
(z) HET-aryl wherein HET is a 5 or 6-membered heteroaromatic ring containing 1-3 heteroatoms selected from O, N and S, wherein said HET-aryl is substituted with 0-5 group selected from halogen, —OH, —OCH₃,
(aa) —C(═O) HET-aryl wherein HET is a 5 or 6-membered heteroaromatic ring containing 1-3 heteroatoms selected from O, N and S, wherein said HET-aryl is substituted with 0-5 group selected from halogen, —OH, —OCH₃,
(bb) cinnamon acyl substituted with 0-5 group selected from halogen, —OH, —OCH₃, and
(cc) —H.

According to an embodiment, there is provided a molecule consisting of:

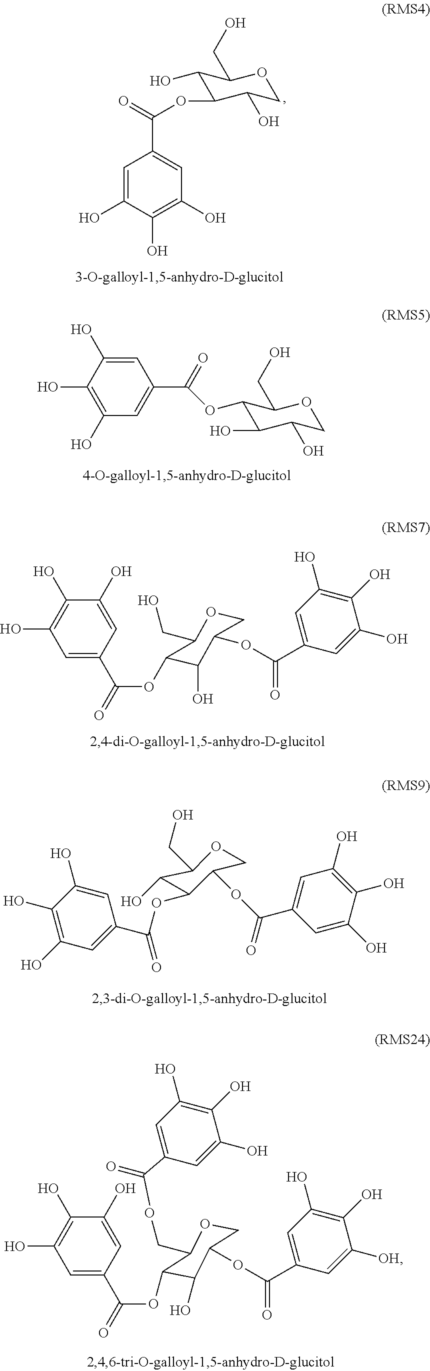 [SMILES: O=C(OC1C(O)COC(CO)C1O)c1cc(O)c(O)c(O)c1 ; O=C(OC1C(CO)OCC(O)C1O)c1cc(O)c(O)c(O)c1 ; O=C(OC1COC(CO)C(OC(=O)c2cc(O)c(O)c(O)c2)C1O)c1cc(O)c(O)c(O)c1 ; O=C(OC1COC(CO)C(O)C1OC(=O)c1cc(O)c(O)c(O)c1)c1cc(O)c(O)c(O)c1 ; O=C(OCC1OCC(OC(=O)c2cc(O)c(O)c(O)c2)C(O)C1OC(=O)c1cc(O)c(O)c(O)c1)c1cc(O)c(O)c(O)c1]

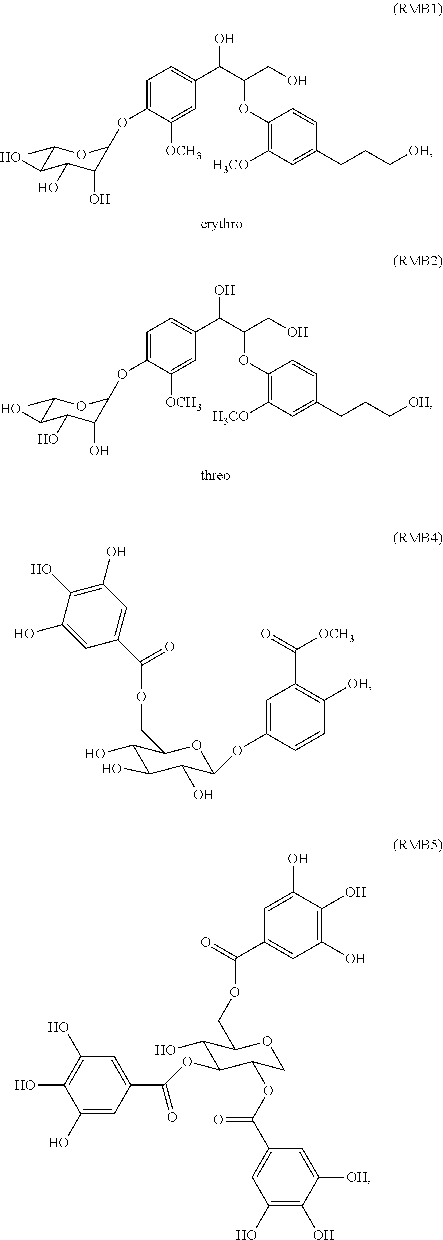 [SMILES: COc1cc(CCCO)ccc1OC(CO)C(O)c1ccc(OC2OC(C)C(O)C(O)C2O)c(OC)c1 ; COc1cc(CCCO)ccc1OC(CO)C(O)c1ccc(OC2OC(C)C(O)C(O)C2O)c(OC)c1 ; COC(=O)c1cc(OC2OC(COC(=O)c3cc(O)c(O)c(O)c3)C(O)C(O)C2O)ccc1O ; O=C(OCC1OCC(OC(=O)c2cc(O)c(O)c(O)c2)C(OC(=O)c2cc(O)c(O)c(O)c2)C1O)c1cc(O)c(O)c(O)c1]

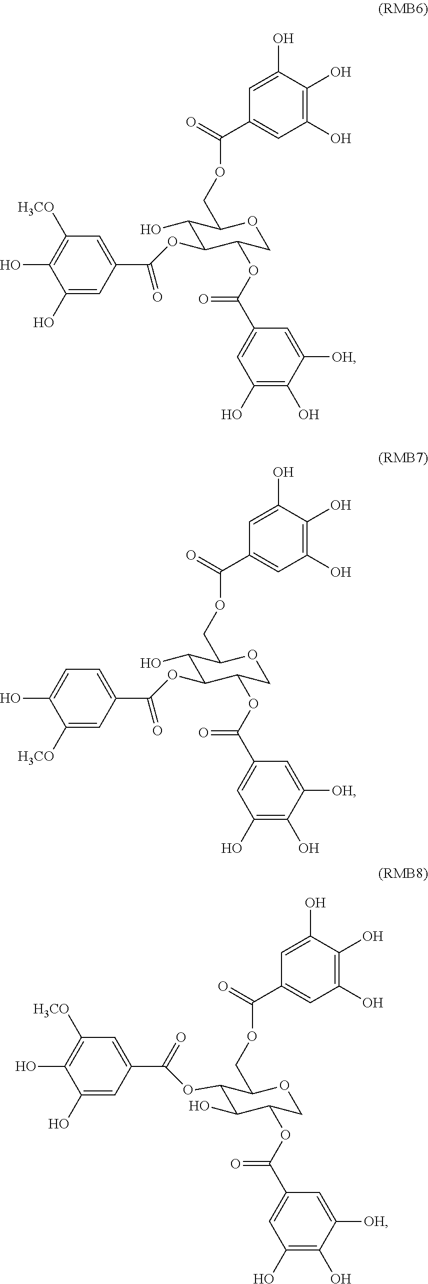 [SMILES: COc1cc(C(=O)OC2C(OC(=O)c3cc(O)c(O)c(O)c3)COC(COC(=O)c3cc(O)c(O)c(O)c3)C2O)cc(O)c1O ; COc1cc(C(=O)OC2C(OC(=O)c3cc(O)c(O)c(O)c3)COC(COC(=O)c3cc(O)c(O)c(O)c3)C2O)ccc1O ; COc1cc(C(=O)OC2C(COC(=O)c3cc(O)c(O)c(O)c3)OCC(OC(=O)c3cc(O)c(O)c(O)c3)C2O)cc(O)c1O]

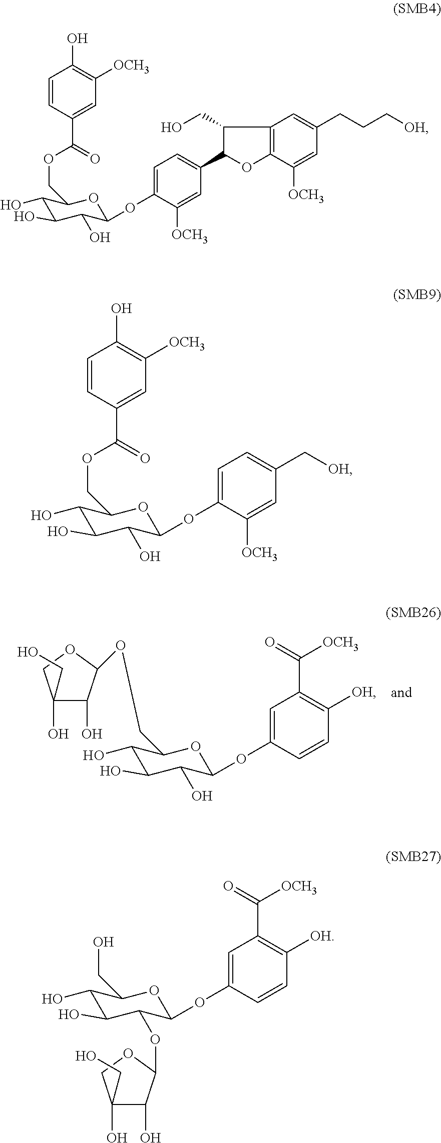

According to an embodiment, there is provided a method of treating an ailment comprising treating a subject with a therapeutically effective amount of a compound according to the present invention.

The ailment may be a diabetes, a cancer, an arthritis, a micro-organism infection, a neurodegenerative disease, an inflammatory disease, an oxidative stress related disease, a heart disease, Alzheimer's diseases, a liver disorder a metabolic syndrome, a damaged hepatic function, a hepatic and liver dyslipidemia, a hepatitis, a liver cancer, an atherosclerosis, a hypertension, a skin disease, an eczema, and a psoriasis.

According to an embodiment, there is provided a use of a compound according to the present invention for the preparation of a medicament for the treatment of an ailment.

According to the present invention, there is provided a use of a compound according to the present invention for the treatment of an ailment.

The ailment may be chosen from a diabetes, a cancer, an arthritis, a micro-organism infection, a neurodegenerative disease, an inflammatory disease, an oxidative stress related disease, a heart disease, Alzheimer's diseases, a liver disorder a metabolic syndrome, a damaged hepatic function, a hepatic and liver dyslipidemia, a hepatitis, a liver cancer, an atherosclerosis, a hypertension, a skin disease, an eczema, and a psoriasis.

The following terms are defined below.

The term “sugar plant” is intended to mean any plant used in the production of sugar. Such plants include, without limitation, maple tree, birch tree, sugar cane, sugar beet and agave, palm tree, among others.

The expressions “any variety of maple tree” or “an Acer tree” is intended to mean a maple tree of a species known to date, such as Acer nigrum, Acer lanum, Acer acuminatum, Acer albopurpurascens, Acer argutum, Acer barbinerve, Acer buergerianum, Acer caesium, Acer campbeffii, Acer campestre, Acer capillipes, Acer cappadocicum, Acer carpinifolium, Acer caudatifolium, Acer caudatum, Acer cinnamomifolium, Acer circinatum, Acer cissifolium, Acer crassum, Acer crataegifolium, Acer davidii, Acer decandrum, Acer diabolicum, Acer distylum, Acer divergens, Acer erianthum, Acer erythranthum, Acer fabri, Acer garrettii, Acer glabrum, Acer grandidentatum, Acer griseum, Acer heldreichii, Acer henryi, Acer hyrcanum, Acer ibericum, Acer japonicum, Acer kungshanense, Acer kweilinense, Acer laevigatum, Acer laurinum, Acer lobelii, Acer lucidum, Acer macrophyllum, Acer mandshuricum, Acer maximowiczianum, Acer miaoshanicum, Acer micranthum, Acer miyabei, Acer mono, Acer mono×Acer truncatum, Acer monspessulanum, Acer negundo, Acer ningpoense, Acer nipponicum, Acer oblongum, Acer obtusifolium, Acer oliverianum, Acer opalus, Acer palmatum, Acer paxii, Acer pectinatum, Acer pensylvanicum, Acer pentaphyllum, Acer pentapomicum, Acer pictum, Acer pilosum, Acer platanoides, Acer poliophyllum, Acer pseudoplatanus, Acer pseudosieboldianum, Acer pubinerve, Acer pycnanthum, Acer rubrum, Acer rufinerve, Acer saccharinum, Acer saccharum, Acer sempervirens, Acer shirasawanum, Acer sieboldianum, Acer sinopurpurescens, Acer spicatum, Acer stachyophyllum, Acer sterculiaceum, Acer takesimense, Acer tataricum, Acer tegmentosum, Acer tenuifolium, Acer tetramerum, Acer trautvetteri, Acer triflorum, Acer truncatum, Acer tschonoskii, Acer turcomanicum, Acer ukurunduense, Acer velutinum, Acer wardii, Acer x peronai, Acer x pseudoheldreichii or any new species not yet known.

The expression “maple-derived” or “maple tree-derived” is intended to mean that the product is derived from any parts (such as bark, leaves, branches, roots, fruits etc.) or any fluid (such as sap) of a member of the Acer genus, as well as extracts obtained from these parts or fluids.

The expression “high pressure processing” is intended to mean the use of physical pressure rather than heat, chemical or irradiation.

The term “extract” is intended to mean any substance made by extracting a part of a raw material (e.g. plant material and/or fluids as defined herein). The extraction method may be by using a solvent such as ethanol or water, or from pulverization, atomization, crystallization, lyophilization, centrifugation, etc of raw materials and/or fluids. Extracts may in solid (e.g. powder) form, semi-solid, semi-liquid, or liquid form. For example, extracts may be from non-concentrated or concentrated sap, a samara fruit, a samara seed, a stems of leaf, a stem of a samara, a twig, a root, a leaf, a bark, a heartwood, a sapwood, a whole branch, a bark of a branch, a wood of a branch, a sugar, a syrup, a syrup extract, a syrup-derived product, a rejection of syrup or syrup-derived product production, a residue of syrup or syrup-derived product production, or combinations thereof.

Features and advantages of the subject matter hereof will become more apparent in light of the following detailed description of selected embodiments, as illustrated in the accompanying figures. As will be realized, the subject matter disclosed and claimed is capable of modifications in various respects, all without departing from the scope of the claims. Accordingly, the drawings and the description are to be regarded as illustrative in nature, and not as restrictive and the full scope of the subject matter is set forth in the claims.

### BRIEF DESCRIPTION OF THE DRAWINGS

Further features and advantages of the present disclosure will become apparent from the following detailed description, taken in combination with the appended drawings, in which:

FIG. 1 shows yeast α-glucosidase inhibition of different phenolic-enriched maple syrup extracts, MS-EtOAc and MS-BuOH, standardized to the same phenolic content.

FIG. 2 shows rat α-glucosidase inhibitory activity of different phenolic-enriched maple syrup extracts, MS-EtOAc and MSBuOH, standardized to the same phenolic content.

FIG. 3 shows porcine α-amylase inhibitory activity of different phenolic-enriched maple syrup extracts, MS-EtOAc and MSBuOH, standardized to the same phenolic content.

FIG. 4 shows HPLC-UV chromatograms of the different phenolic-enriched maple syrup extracts, MS-EtOAc and MS-BuOH, shown in panels A and B respectively.

FIG. 5 shows total phenolic content of sugar and red maple leaves collected in summer and fall (Values with different letters are significantly different, p<0.05).

FIG. 6 shows HPLC phenolic profiles of leaves from red maple summer (A), red maple fall (B), sugar maple summer (C) and sugar maple fall (D). Chromatogram are extracted at 360 nm.

FIG. 7 shows dose-dependent rat α-glucosidase inhibition of sugar and red maple leaves collected in summer and fall (Values with different letters are significantly different, p<0.05).

FIG. 8 shows dose-dependent porcine α-amylase inhibition of sugar and red maple leaves collected in summer and fall (Values with different letters are significantly different, p<0.05).

FIG. 9 shows the chemical structures of ginnalin-A (1), ginnalin-B (2) and ginnalin-C (3) isolated from Red-leaf maple twigs/stems and used for standardization of the maple plant part extracts. The molecular weights of compounds I-3 are 468, 316, and 316 g/mol, respectively.

FIG. 10 shows HPLC-UV chromatograms of maple plant part extracts showing the presence of ginnalins A-C in the Red-leaf maple (FIG. 2A) and Sugar maple (FIG. 2B) species. HPLC profiles are as follows: a=leaf extracts; b=stem/twigs; c=bark extracts; d=sapwood extracts. Peak 1 (T_{R} of 26 min)=ginnalin A; peak 2/3 coeluting (T_{R} of 15/16 min)=ginnalins-B and C, respectively. Chromatograms are monitored at a wavelength of 280 nm.

FIG. 11 shows the analysis of cell cycle distribution of cell lines treated with different extracts. Distribution of cells in the G0/G1, S and G2/M phases at 72 h: (A) HCT-116 cells, (B) Caco-2 cells, (C) HT-29 cells, (D) CCD-18Co cells. Data are expressed as mean values±SD (n=3). *p<0.05 (two-tailed t test) indicate a significant difference compared to untreated cells.

FIG. 12 shows the structures of compounds RMS1-13.

FIG. 13 shows the (a) ¹H-¹H COSY (—) and key HMBC correlations (H→C) of RMS4; and (b) ¹H-¹H COSY (—) and key HMBC correlations (H→C) of RMS5.

### DETAILED DESCRIPTION OF THE PREFERRED EMBODIMENTS

In embodiments there are disclosed nutraceutical, functional food, food ingredients and non-food ingredients (ingredient having no nutritional value, but having other effects) composition as well as pure total or partial extracts. Neutraceuticals and functional food are food or food product that provides health and medical benefits, including the prevention and treatment of disease. Products according to the present invention may range from isolated nutrients, dietary supplements and specific diets and herbal products, and processed foods such as cereals, soups, and beverages. The composition of the present invention contain extracts of an Acer tree from sap, samara (including the fruits, the seeds as well as the stem), leaves (including the stem), twigs roots, heartwood and sap wood and/or the bark of the tree, or even maple syrup or maple sugar or maple sap, maple concentrate. The composition of the present invention also encompass synthetic reconstructions of extracts from Acer Saccharum or any other variety of maple tree. The composition of the present invention may be consumed for the prophylaxis of ailments such as diabetes, such as diabetes melitus, cancer, arthritis, micro-organism infection such as bacterial infections, neurodegenerative diseases, inflammatory diseases, oxidative stress related diseases, and heart diseases, as an anti-oxidant, anticancer, neurodegenerative diseases, Alzheimer's disease, liver disorders (such as metabolic syndrome, damaged hepatic function, hepatic and liver dyslipidemia, hepatitis, liver cancer), atherosclerosis, hypertension, and skin diseases (such as eczema, psoriasis and the likes).

In embodiments there is disclosed an Acer tree essential oil composition. An essential oil is a concentrated, hydrophobic liquid containing volatile aroma compounds from plants. An oil is essential in the sense that it carries a distinctive scent, or essence, of the plant. Essential oils do not as a group need to have any specific chemical properties in common, beyond conveying characteristic fragrances. Essential oils of the present invention are generally extracted by distillation, but they may be extracted by expression (i.e. pressing), or solvent extraction. The essential oil of the present invention may be used in perfumes, cosmetics, soap and other products, and even for flavouring food and drink, and for scenting incense and household cleaning products, and for use as anti-foaming agents in the production of maple syrup.

Various essential oils have been used medicinally at different periods in history. The essential oil of the present invention may be used in medical application ranging from skin treatments to remedies for cancer, and as cosmeceutical products (products that provide cosmetic functions as well as pharmaceutical functions), such as exfolients, masks, ointments, lotions, gels, creams, and the like.

The essential oils of the present invention contain the hydrophobic fraction extracted from an Acer tree biomass (sap, samara (including the fruits, the seeds as well as the stem), leaves (including the stem), twigs, roots, heartwood and sap wood, the bark of the tree.). Because of their concentrated nature, the essential oils of the present invention generally should not be applied directly to the skin in their undiluted form. They should rather be diluted in a suitable solvent, such as ethanol, polyethylene glycol, or a pharmaceutically acceptable carrier oil. Carrier oils are used to dilute essential and other oils prior to application. They “carry” the essential oil onto the skin. Suitable carrier oils include but are not limited to sweet almond oil, kukui nut oil, apricot kernel oil, macadamia nut oil, avocado oil, meadowfoam oil, borage seed oil, olive oil, camellia seed oil, peanut oil, cranberry seed oil, pecan oil, evening primrose oil, pomegranate seed oil, fractionated coconut oil, rose hip oil, grapeseed oil, seabuckthorn berry oil, hazelnut oil, sesame oil, hemp seed oil, sunflower oil, jojoba, and watermelon seed oil.

According to an embodiment of the persent invention, the essential oils of the present invention may be used as anti-foaming agents in the production of maple syrup. Other foaming agents are usually used in the production of maple syrup (e.g. vegetal oils certified “biologic”, with the exception of oils from soya, peanuts, nuts, or sesame seeds, due to their known allergenic potential). Thus, according to the present invention, essential oils extracted from maple tree biomass may be employed to replace these vegetal oils.

In embodiments there is disclosed a food product which comprises germinated and/or fermented Acer tree samara (including the fruits and the seeds). The samara from an Acer tree is a fruit in which a flattened wing of fibrous, papery tissue develops from the ovary wall. The samara is comestible and may be germinated to yield germinated an Acer tree samara.

Acer tree samara are produced naturally by maple trees as part of their reproductive cycle each year. They are produced in large quantities, most of which simply fall to the ground and degrade, representing a significant missed economical opportunity for sugar bush operators. Therefore, according to the present invention, Acer tree samara may be incorporated into food product in their germinated form, in food product such as salads accompanied with high quality oils or vinegars, salad sauces or vinaigrettes. They may even be incorporated in stir fries with meat and other vegatables.

According to the present invention the samara may also be marinated prior to consumption in vinegar, for example, or in any other suitable marinating solution which may preserve the marinated samara for extended periods of time.

According to the present invention, the seeds of the samara may also be dessicated to serve as as healthy food. Furthermore, oil may also be extracted from samara to be employed as a food with healthy properties.

In embodiments there is also disclosed a solid sweetening composition for oral consumption containing an Acer tree sugar extract; and other sweeteners. The Acer tree Saccharum sugar extract may be from maple syrup, maple taffy, flakes, maple sugar, maple spread. The sweetening composition may also be prepared from any synthetic source of maple sugars or from synthetic compositions recapitulating natural maple-derived products.

The sweetener may be chosen from a nutritive sweetener and a non-nutritive sweetener. Examples of nutritive sweeteners include but are not limited to honey, birch syrup, pine syrup, hickory syrup, poplar syrup, palm syrup, sugar beet syrup, sorghum syrup, corn syrup, cane syrup, golden syrup, barley malt syrup, a molasse, brown rice syrup, agave nectar, yacon syrup, fructose, maltitol, brown sugar, Okinawa syrup or combinations thereof.

The non-nutritive sweeteners may include but are not limited to adenosine monophosphate, acesulfame potassium, alitame, aspartame, anethole, cyclamate, glycyrrhizin, lo han guo, miraculin, neotame, perillartine, saccharin, selligueain A, a Stevia rebaudiana extract, sucralose, thaumatin neohesperdine DC, thavmatin, brazzein, and inulin. The Stevia rebaudiana extract may include stevioside, rebaudioside A, rebaudioside B, and rebaudioside C.

In use, the selection of certain maple-sugar, for example a maple taffy with other sweetener (e.g. stevia extract) allows for unique organoleptic qualities to be combined into novel combinations of natural sugars. These may present advantageous nutritional and tasteful qualities in a synergistic manner, and may therefore stimulate the individuals in unexpected manners.

In embodiments there is also disclosed an infusion composition for the preparation of a beverage. The infusion composition is provided in a dried form comprising an extract of Acer tree leaves, bark, roots, twigs of leaves or stems of samara, samara (fruits/seeds) and mixture thereof. The composition may also include other herbal component such as tea, and/or a herbal tea. The infusion composition of the present invention may comprise the extract of Acer tree leaves in combination with a number tea such as the following non-limiting examples including Bai Hao Yinzhen tea, Bai Mu Dan tea, Pai Mu Tan tea, Gong Mei tea, Shou Mei tea, White Puerh tea, Ceylon White tea, Darjeeling White tea, Assam White tea, African White tea, Junshan Yinzhen tea, Huoshan Huangya tea, Meng Ding tea, Huangya tea, Da Ye Qing tea, Huang Tang tea, Junshan Yinzhen tea, Longjing tea, Hui Ming tea, Long Ding tea, Hua Ding tea, Qing Ding tea, Gunpowder tea, Bi Luo Chun tea, Rain Flower tea, Shui Xi Cui Bo tea, Camellia Sinensis tea, Yu Lu tea, Xin Yang Mao Jian tea, Chun Mee tea, Gou Gu Nao tea, Yun Wu tea, Da Fang tea, Huangshan Maofeng tea, Lu'An Guapian tea, Hou Kui tea, Tun Lu tea, Huo Qing tea, Wuliqing tea, Hyson tea, Zhu Ye Qing tea, Meng Ding Gan Lu tea, Genmaicha tea, Gyokuro tea, Kabusecha tea, Sencha tea, Fukamushicha tea, Tamaryokucha tea, Bancha tea, Kamairicha tea, Kukicha tea, Mecha tea, Konacha tea, Matcha tea, Genmaicha tea, Bancha tea, Höjicha tea, Tencha tea, Aracha tea, Shincha tea, funmatsucha tea or combinations thereof.

The infusion composition may also include herbal tea which include but are not limited to anise tea, artichoke tea, roasted barley tea, bee balm tea, boldo tea, cannabis tea, catnip tea, Ilex causue leaves tea, cinnamon tea, coffee leaves tea, coffee cherry tea, Cerasse tea, dried chamomile blossoms tea, chrysanthemum tea, citrus peel tea, bergamot tea, orange peel tea, dandelion tea, dill tea, echinacea tea, essiac tea, fennel tea, gentian tea, ginger root tea, ginseng tea, hawthorn tea, hibiscus tea, rose hip tea, honeybush tea, horehound tea, hydrangea tea, Jiaogulan tea, Kapor tea, Kava root tea, Ku Ding tea, Labrador tea, Lapacho tea, lemon balm tea, lemon grass tea, licorice root tea, lime blossom tea, yerba mate tea, mate de coca tea, mint tea, european mistletoe tea, neem leaf tea, nettle leaf tea, asiatic pennywort leaf tea, pennyroyal leaf tea, pine tea, red raspberry leaf tea, scorched rice tea, rooibos tea, roselle petals, rosemary memory herb tea, sage tea, skullcap tea, serendib tea, sobacha, spicebush leaf tea, spruce tea, staghorn sumac fruit tea, stevia tea, St. John's Wort tea, tulsi tea, uncaria tomentosa, valerian tea, Verbena tea, vetiver tea, roasted wheat tea, wax gourd tea, Wong Logat tea, woodruff tea, yarrow tea, yerba mate tea, yuen kut lam kam wo tea or combinations thereof.

According to an embodiment, the infusion composition according to the present invention may be infused with a maple tree based matrix, such as a maple tree sap, a concentrated maple tree water, a maple tree syrup. The maple tree based matrix is believed to act as a nutriprotective carrier.

In embodiments, there is also disclosed a compound of formula (I), or a pharmaceutically acceptable salt thereof:

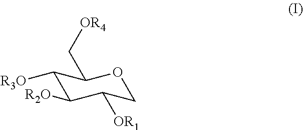

where R₁ and R₄ are each independently chosen from
(a) C₁₋₃ alkyl substituted with 0-3 groups selected from halogen, —OH, —OCH₃,
(b) —C(═O)H,
(c) —C(═O)C₁₋₃ alkyl substituted with 0-3 groups selected from halogen, —OH, —OCH₃,
(d) —CN,
(e) —HC—NOH,
(f) —(CH₃)C═NOH,
(g) —HC═NOC₁₋₃ alkyl substituted with 0-3 groups selected from halogen, —OH, —OCH₃,
(h) —(CH₃)C═NOC₁₋₃ alkyl substituted with 0-3 groups selected from halogen, —OH, —OCH₃,
(i) —C(═O)OC₁₋₃ alkyl substituted with 0-3 groups selected from halogen, —OH, —OCH₃,
(j) aryl substituted with 0-5 group selected from halogen, —OH, —OCH₃,
(k) —C(═O) aryl substituted with 0-5 group selected from halogen, —OH, —OCH₃,
(l) HET-aryl wherein HET is a 5 or 6-membered heteroaromatic ring containing 1-3 heteroatoms selected from O, N and S, wherein said HET-aryl is substituted with 0-5 group selected from halogen, —OH, —OCH₃,
(m) —C(═O)HET-aryl wherein HET is a 5 or 6-membered heteroaromatic ring containing 1-3 heteroatoms selected from O, N and S, wherein said HET-aryl is substituted with 0-5 group selected from halogen, —OH, —OCH₃,
(n) cinnamon acyl substituted with 0-5 group selected from halogen, —OH, —OCH₃, and
R₂ and R₃ are each independently chosen from
(o) C₁₋₃ alkyl substituted with 0-3 groups selected from halogen, —OH, —OCH₃,
(p) —C(═O)H,
(q) —C(═O)C₁₋₃ alkyl substituted with 0-3 groups selected from halogen, —OH, —OCH₃,
(r) —CN,
(s) —HC—NOH,
(t) —(CH₃)C═NOH,
(u) —HC═NOC₁₋₃ alkyl substituted with 0-3 groups selected from halogen, —OH, —OCH₃,
(v) —(CH₃)C═NOC₁₋₃ alkyl substituted with 0-3 groups selected from halogen, —OH, —OCH₃,
(w) —C(═O)OC₁₋₃ alkyl substituted with 0-3 groups selected from halogen, —OH, —OCH₃,
(x) aryl substituted with 0-5 group selected from halogen, —OH, —OCH₃,
(y) —C(═O) aryl substituted with 0-5 group selected from halogen, —OH, —OCH₃,
(z) HET-aryl wherein HET is a 5 or 6-membered heteroaromatic ring containing 1-3 heteroatoms selected from O, N and S, wherein said HET-aryl is substituted with 0-5 group selected from halogen, —OH, —OCH₃,
(aa) —C(═O)HET-aryl wherein HET is a 5 or 6-membered heteroaromatic ring containing 1-3 heteroatoms selected from O, N and S, wherein said HET-aryl is substituted with 0-5 group selected from halogen, —OH, —OCH₃,
(bb) cinnamon acyl substituted with 0-5 group selected from halogen, —OH, —OCH₃, and
(cc) —H.

In embodiments, there is also disclosed molecules consisting of:

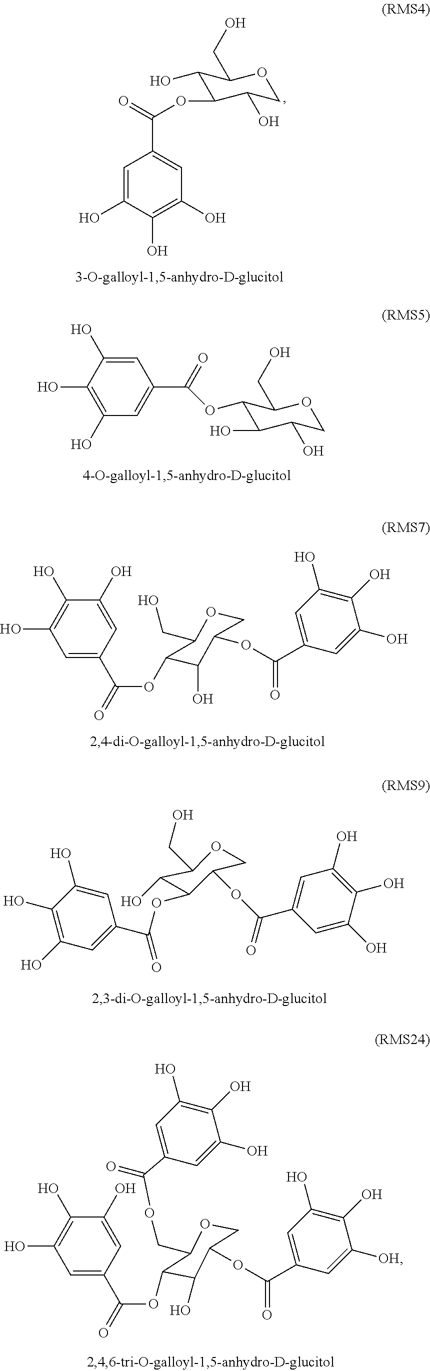 [SMILES: O=C(OC1C(O)COC(CO)C1O)c1cc(O)c(O)c(O)c1 ; O=C(OC1C(CO)OCC(O)C1O)c1cc(O)c(O)c(O)c1 ; O=C(OC1COC(CO)C(OC(=O)c2cc(O)c(O)c(O)c2)C1O)c1cc(O)c(O)c(O)c1 ; O=C(OC1COC(CO)C(O)C1OC(=O)c1cc(O)c(O)c(O)c1)c1cc(O)c(O)c(O)c1 ; O=C(OCC1OCC(OC(=O)c2cc(O)c(O)c(O)c2)C(O)C1OC(=O)c1cc(O)c(O)c(O)c1)c1cc(O)c(O)c(O)c1]

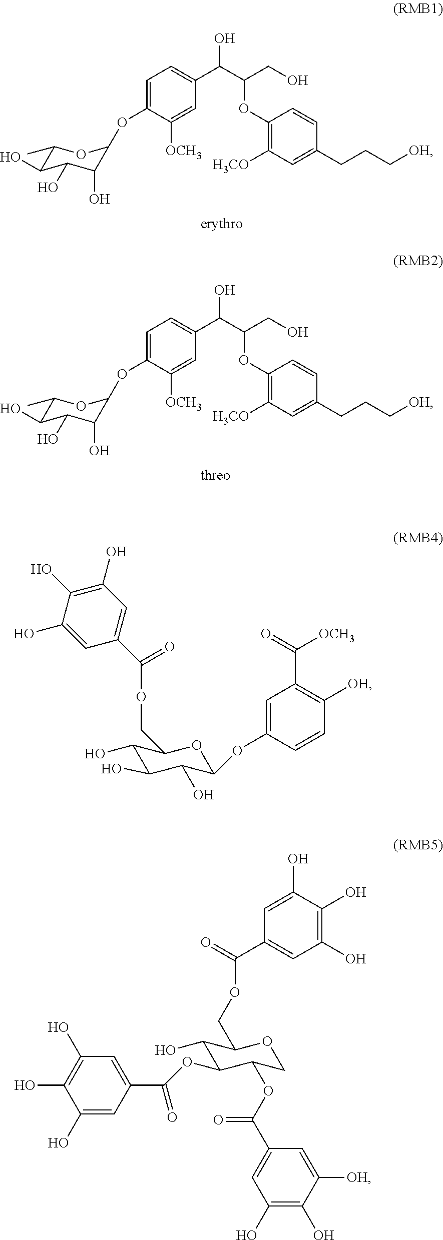 [SMILES: COc1cc(CCCO)ccc1OC(CO)C(O)c1ccc(OC2OC(C)C(O)C(O)C2O)c(OC)c1 ; COc1cc(CCCO)ccc1OC(CO)C(O)c1ccc(OC2OC(C)C(O)C(O)C2O)c(OC)c1 ; COC(=O)c1cc(OC2OC(COC(=O)c3cc(O)c(O)c(O)c3)C(O)C(O)C2O)ccc1O ; O=C(OCC1OCC(OC(=O)c2cc(O)c(O)c(O)c2)C(OC(=O)c2cc(O)c(O)c(O)c2)C1O)c1cc(O)c(O)c(O)c1]

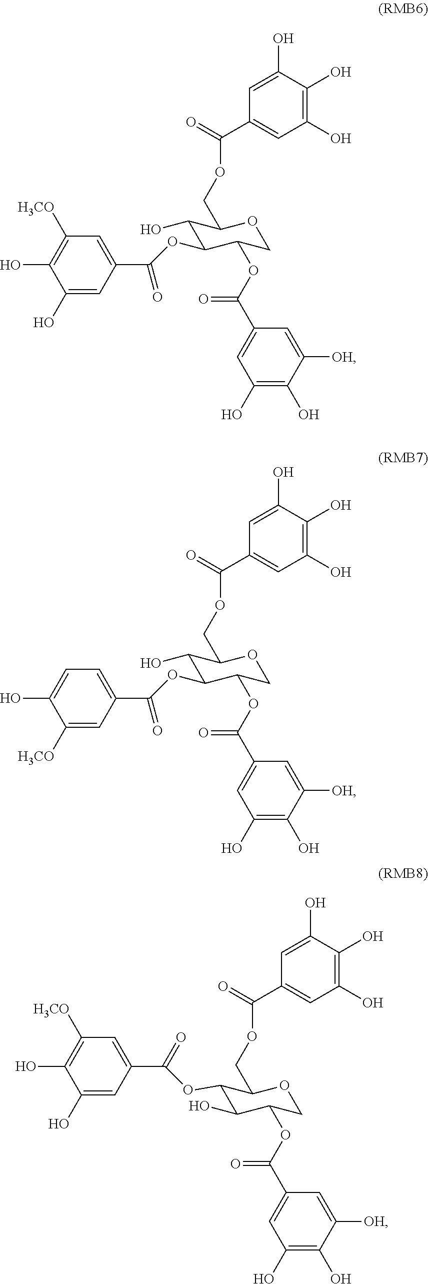 [SMILES: COc1cc(C(=O)OC2C(OC(=O)c3cc(O)c(O)c(O)c3)COC(COC(=O)c3cc(O)c(O)c(O)c3)C2O)cc(O)c1O ; COc1cc(C(=O)OC2C(OC(=O)c3cc(O)c(O)c(O)c3)COC(COC(=O)c3cc(O)c(O)c(O)c3)C2O)ccc1O ; COc1cc(C(=O)OC2C(COC(=O)c3cc(O)c(O)c(O)c3)OCC(OC(=O)c3cc(O)c(O)c(O)c3)C2O)cc(O)c1O]

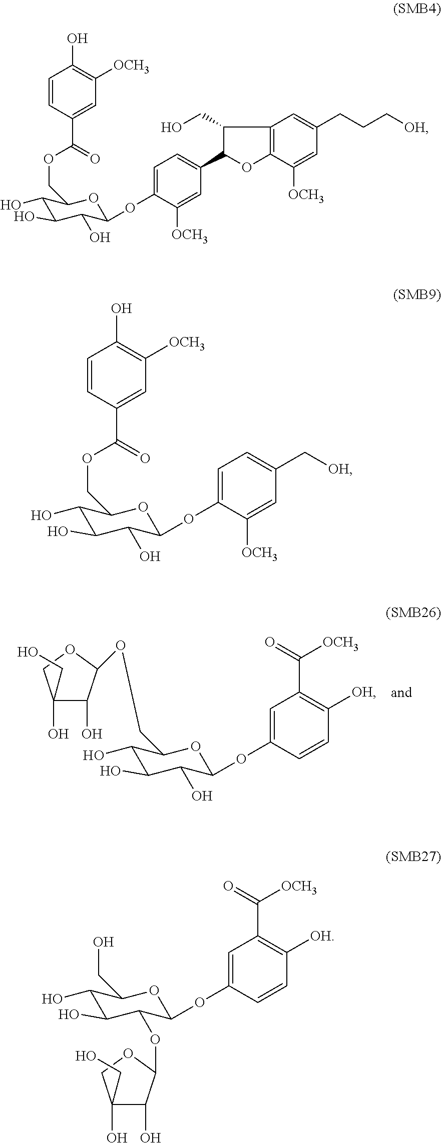

The compounds of formula (I) as well as the molecules are believed to be useful for the treatment of ailments, such as diabetes, cancers, arthritis, micro-organism infections, neurodegenerative diseases, inflammatory diseases, oxidative stress related diseases, heart diseases, Alzheimer's diseases, liver disorders, a metabolic syndromes, damaged hepatic functions, hepatic and liver dyslipidemias, hepatitis, liver cancers, atherosclerosis, hypertensions, skin diseases, eczema, and psoriasis.

The present invention will be more readily understood by referring to the following examples which are given to illustrate the invention rather than to limit its scope.

### Example 1

### In Vitro Evaluation of Phenolic-Enriched Maple Syrup Extracts for Inhibition of Carbohydrate Hydrolyzing Enzymes Relevant to Type 2 Diabetes Management

The objective of the current example is to evaluate the ability of phenolic-enriched extracts of Canadian maple syrup, namely ethyl acetate (MS-EtOAc) and butanol (MS-BuOH), to inhibit carbohydrate hydrolyzing enzymes relevant to type 2 diabetes management.

Extracts are standardized to phenolic contents by the Folin-Ciocalteau method and assayed for yeast α-glucosidase inhibitory activities. On normalization to phenolic content, MS-BuOH exhibited higher inhibitory activity than MS-EtOAc (IC₅₀=68.38 and 107.9 μg phenolics, respectively). The extracts are further assayed for inhibition of porcine α-amylase and rat α-glucosidase enzymes. MSBuOH exhibited higher rat α-glucosidase and porcine α-amylase inhibitory activities (IC₅₀=135 and 103 μg phenolics, respectively) than MS-EtOAC extract (IC₅₀>187 μg phenolics in both assays). These results suggest that maple syrup extracts have potential for phenolic-mediated type 2 diabetes management, with the MS-BuOH phenolic-enriched fraction having highest bioactivity.

Type 2 diabetes accounts for about 90-95% of all diagnosed cases of diabetes in adults (Centers for Disease Control and Prevention, 2010). Worldwide, at least 220 million people have diabetes and this figure is estimated to double by 2030 (World Health Organization, 2010). In the United States alone, in 2007, 23.7 million people (10% of American adults) had diabetes and this figure is expected to jump to 33% (i.e. one-third of all American adults) by 2050 (Centers for Disease Control and Prevention, 2010). Remarkably, the cost to manage diabetes by Americans in 2007 is $174 billion and this figure is expected to skyrocket based on the CDC's latest estimates (Centers for Disease Control and Prevention, 2010). Thus type 2 diabetes poses a major public health challenge with significant health care costs and burden.

The major source of blood glucose is hydrolyzed dietary carbohydrates such as starches. Dietary carbohydrates are hydrolyzed by pancreatic α-amylase with absorption aided by α-glucosidases in order to be absorbed by the small intestine (Elsenhans & Caspary, 1987). It is believed that inhibition of these enzymes can be an important strategy for management of type 2 diabetes (Krentz & Bailey, 2005).

Large polysaccharides (starch) are broken down by α-amylase which acts upon their internal bonds. Natural α-amylase inhibitors offer an attractive therapeutic approach to the treatment of postprandial hyperglycemia by ultimately decreasing glucose release from starch. The α-glucosidase enzyme catalyzes the final step of glucose absorption in the small intestine during the digestive process of carbohydrates, and hence α-glucosidase inhibitors could retard the rapid utilization of dietary carbohydrates and suppress postprandial hyperglycemia (Watanabe, Kawabata, Kurihara, & Niki, 1997). The clinical use of drug-inhibitors such as acarbose has been attempted for diabetic or obese patients. Acarbose has been shown to effectively reduce the intestinal absorption of sugars in humans (Cheng & Fantus, 2005; Jenkins et al., 1981). Recently, it has been shown that plant derived phenolics play a role in mediating α-glucosidase and α-amylase inhibition and thus have potential to contribute to the management of type 2 diabetes (Apostolidis, Kwon, & Shetty, 2006; Hogan et al., 2010; Kwon, Apostolidis, Kim, & Shetty, 2007; Kwon, Vattem, & Shetty, 2006).

Maple syrup is a natural sweetener and is the largest commercially available food product that is totally derived from the sap of deciduous trees. It is obtained by concentrating the sap collected from certain maple species including the sugar maple tree (Acer saccharum Marsh.) which is native to North America (Ball, 2007; Perkins & van der Berg, 2009). Maple syrup is produced primarily in North America with the vast majority of the world's supply coming from Canada (85%; primarily Quebec), followed by United States (Perkins & van der Berg, 2009). Previous reports have shown that maple syrup contains a wide variety of phenolic phytochemicals (Abou-Zaid, Nozzolillo, Tonon, Coppens, & Lombardo, 2008; Filipie & Underwood, 1964; Kermasha, Goetghebeur, & Dumont, 1995; Li & Seeram, 2010; Potter & Fagerson, 1992), which may have positive effects on human health. Recently, phenolic-enriched extracts of maple syrup are shown to have antioxidant, anti-mutagenic and human cancer cell anti-proliferative properties (Legault, Girard-Lalancette, Grenon, Dussault, & Pichette, 2010; Li & Seeram, 2010; Theriault, Caillet, Kermasha, & Lacroix, 2006). In addition, Honma, Koyama, and Yazawa (2010) reported that A. saccharum leaf extracts had phenolic-mediated potential for type 2 diabetes management via inhibition of the carbohydrate hydrolyzing enzyme-glucosidase.

A comprehensive evaluation of different sweeteners (including sugar cane, brown sugar, date sugar and corn syrup, among others) showed a phenolic-dependent α-glucosidase inhibitory activity, however, maple syrup is not evaluated (Ranilla, Kwon, Genovese, Lajolo, & Shetty, 2008). In addition, Ranilla et al. (2008) used crude water extracts, without previous removal of sugars that could possibly lead to substrate-related inhibition in the α-glucosidase bioassay. The objective of the current document is to evaluate the type 2 diabetes management potential, via inhibition of carbohydrate hydrolyzing enzymes, of phenolic-enriched extracts of maple syrup (namely, ethyl acetate and butanol) in which sugars are previously removed. For the identification of potential maple syrup compounds having type 2 diabetes management potential, it is important to determine the effects of different phenolic-enriched maple syrup extracts. This is because various organic solvents used for extraction of maple syrup will result in a different profile of phenolic compounds (Li & Seeram, 2010). Maple syrup is a plant-derived natural sweetener that contains a wide variety of natural phenolic compounds beyond its sugars (predominantly as sucrose). Thus, identification of the relevant maple syrup-derived compounds for type 2 diabetes management requires the evaluation of various maple syrup extracts that contains different phenolic profiles. In the present document, the potential of phenolic-standardized maple syrup extracts for type 2 diabetes management is reported for the first time. A clear knowledge of the relevant maple syrup compounds that contribute towards sugar absorption management in the gastrointestinal tract could potentially lead to the design of natural sweeteners with lower glycemic index.

Materials and Methods

Maple syrup (grade C) is provided by the Federation of Maple Syrup Producers of Quebec (Canada). The syrup is kept frozen in the laboratory until extraction. High performance liquid chromatography (HPLC) is performed on a Hitachi Elite LaChrom system consisting of a L2130 pump, L-2200 autosampler, and a L-2455 Diode Array Detector all operated by EZChrom Elite software. All solvents are of either ACS or HPLC grade and are purchased from Wilkem Scientific (Pawtucket, R.I.). α-Amylase (porcine pancreatic, EC 3.2.1.1), α-glucosidase (yeast, EC 3.2.1.20) and rat intestinal powder are purchased from Sigma-Aldrich (St. Louis, Mo.). Unless otherwise specified, all other chemicals are purchased from Sigma-Aldrich.

Sample Preparation

Preparation of phenolic-enriched extracts of maple syrup has been previously reported (Li & Seeram, 2010). Briefly, maple syrup (20 L) is subjected to liquid-liquid partitioning with ethyl acetate (10 L×3) and n-butanol (10 L×3) successively. The combined ethyl acetate extracts are dried under reduced pressure and accurate weights are obtained as 4.71 g (MS-EtOAc). After concentration, remaining sugars are removed from the combined n-butanol extracts (108 g) by further extraction with methanol (100 mL×3) at room temperature to afford 57 g (MS-BuOH). All samples are standardized to solid content of 125 mg/mL for further assaying.

Standardization of Maple Syrup Extracts

Standardization based on total phenolic content. Total phenolic content is determined according to the Folin-Ciocalteu's method and are measured as gallic acid equivalents (GAEs) as previously reported (Singleton & Esau, 1969). Briefly, the extracts are appropriately diluted with methanol/H₂O (1:1, v/v), and 200 μL of sample is incubated with 3 mL of methanol/H₂O (1:1, v/v) and 200 μL of Folin-Ciocalteau reagent for 10 min at 25° C. After this, 600 μL of 20% Na₂CO₃ solution is added to each tube and vortexed. Tubes are further incubated for 20 min at 40° C. After incubation, samples are immediately cooled in an ice bath to room temperature. Samples and standards (gallic acid) are processed identically and all tests are performed in triplicate. The absorbance is read at 755 nm, and the total phenolic content is calculated from the standard curve obtained from a Spectramax plate reader (Molecular Devices, Sunnyvale, Calif., USA).

Standardization Based on HPLC-UV Analyses.

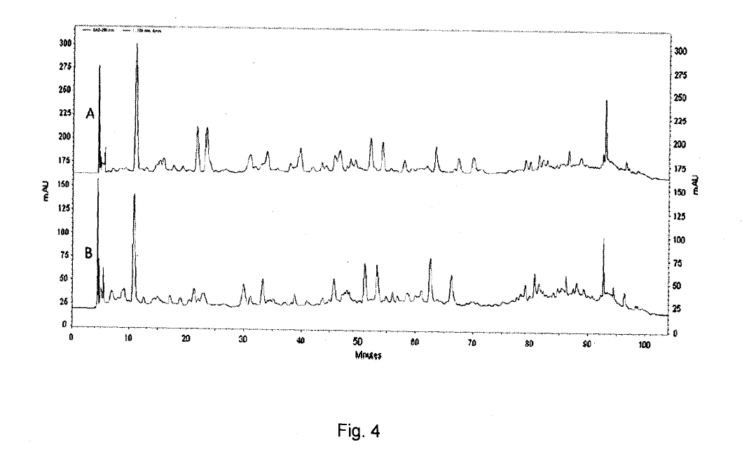

The HPLC-UV analyses are carried out as previously reported (Li & Seeram, 2010). Briefly, a Luna C18 column (250×4.6 mm i.d., 5 μM; Phenomenex) with a flow rate at 0.75 ml/min and injection volume of 20 μL for both extracts is used. The extracts are dissolved in dimethylsulphoxide (DMSO) and analyzed at equivalent phenolic contents. A gradient solvent system consisting of solvent A (0.1% aqueous trifluoroacetic acid) and solvent B (methanol, MeOH) is used as follows: 0-10 min, from 10% to 15% B; 10-20 min, 15% B; 20-40 min, from 15% to 30% B; 40-55 min, from 30% to 35% B; 55-65 min, 35% B; 65-85 min, from 35% to 60% B; 85-90 min, from 60% to 100% B; 90-93 min, 100% B; 93-94 min, from 100% to 10% B; 94-104 min, 10% B. FIG. 4 shows the HPLC-UV profiles at 280 nm of the maple syrup extracts as follows: MS-EtOAc (panel A) and MS-BuOH (panel B), respectively.

Antioxidant Activity by 1,1-diphenyl-2-picrylhydrazyl (DPPH) Radical Inhibition Assay

The antioxidant potentials of MS-EtOAC and MS-BuOH are determined on the basis of the ability to scavenge the DPPH radicals as previously described (Nanjo et al., 1996). The DPPH radical scavenging activity of ascorbic acid (vitamin C) and the synthetic commercial antioxidant, butylated hydroxytoluene (BHT), are also assayed as positive controls. The assay is conducted in a 96-well format using serial dilutions of 100 μL aliquots of test compounds (ranging from 2500 to 26 μg/mL), ascorbic acid (1000-10.4 μg/mL), and BHT (250,000-250 μg/mL). Then DPPH (150 μL) is added to each well to give a final DPPH concentration of 137 Absorbance is read after 30 min at 515 nm, and the scavenging capacity (SC) is calculated as SC %=[(A₀−A₁/A₀]×100, where A₀ is the absorbance of the reagent blank and A₁ is the absorbance with test samples. All tests are performed in triplicate. IC₅₀ values denote the concentration of sample required to scavenge 50% DPPH radicals.

Carbohydrate Hydrolysis Enzyme Inhibition Assays

Since phenolic phytochemicals have been extensively shown to have α-glucosidase inhibitory activity (Apostolidis & Lee, 2010; Apostolidis et al., 2006; Hogan et al. 2010; Honma et al., 2010; Khan, Tiwari, Ahmad, Srivastava, & Tripathi, 2004; Kwon et al., 2006, 2007), the extracts are standardized to a phenolic content of 3.75 mg GAE/mL to be evaluated on the same basis using the assay below.

Yeast α-glucosidase Inhibition Assay.

A mixture of 50 μL of extract and 100 μL of 0.1M phosphate buffer (pH 6.9) containing yeast α-glucosidase solution (1.0 U/ml) is incubated in 96 well plates at 25° C. for 10 min. After pre-incubation, 50 μL of 5 mM p-nitrophenyl-α-D-glucopyranoside solution in 0.1M phosphate buffer (pH 6.9) is added to each well at timed intervals. The reaction mixtures is incubated at 25° C. for 5 min. Before and after incubation, absorbance is recorded at 405 nm by a micro-plate reader (VMax, Molecular Device Co., Sunnyvale, Calif., USA) and compared to that of the control which had 50 μL buffer solution in place of the extract. The α-glucosidase inhibitory activity is expressed as inhibition % and is calculated as follows:

$$\% Inhibition = ( \frac{Δ {Abs}_{control} - Δ {Abs}_{sample}}{Δ Abscontrol} ) \times 100$$

Rat α-glucosidase Inhibition Assay.

To validate the yeast α-glucosidase inhibition results, the rat α-glucosidase assay with the fractions that resulted at the highest inhibition is used Rat intestinal α-glucosidase assay is referred to the method of Kwon et al. (2007) with a slight modification. A total of 1 g of rat-intestinal acetone powder is suspended in 10 mL of 0.9% saline, and the suspension is sonicated twelve times for 30 s at 4° C. After centrifugation (10,000 g, 30 min, 4° C.), the resulting supernatant is used for the assay. Sample solution (50 μL) and 0.1M phosphate buffer (pH 6.9, 100 μL) containing α-glucosidase solution is incubated at 25° C. for 10 min. After preincubation, 5 mM p-nitrophenyl-α-D-glucopyranoside solution (50 μL) in 0.1M phosphate buffer (pH 6.9) is added to each well at timed intervals. The reaction mixtures are incubated at 25° C. for 30 min and readings are recovered every 5 min. Before and after incubation, absorbance is read at 405 nm and compared to a control which has 50 μL of buffer solution in place of the extract by micro-plate reader. The α-glucosidase inhibitory activity is expressed as inhibition % and is calculated as follows:

$$\% Inhibition = ( \frac{Δ {Abs}_{control} - Δ {Abs}_{sample}}{Δ Abscontrol} ) \times 100$$

Porcine α-amylase Inhibition Assay.

A mixture of 50 μL of extract or acarbose and 50 μL 0.02 M sodium phosphate buffer (pH 6.9 with 0.006 M sodium chloride) containing α-amylase solution (13 U/mL) is incubated at 25° C. for 10 min using an 1.5 mL Eppendorf tube. After pre-incubation, 50 μL 1% soluble starch solution in 0.02 M sodium phosphate buffer (pH 6.9 with 0.006 M NaCl) is added to each well at timed intervals. The reaction mixtures are then incubated at 25° C. for 10 min followed by addition of 1 mL dinitrosalicylic acid colour reagent. The test tubes are then placed in a boiling water bath for 10 min to stop the reaction and cooled to room temperature. The reaction mixture is then diluted with 1 mL distilled water and absorbance is read at 540 nm using a 96-well mircoplate reader.

$$\% Inhibition = ( \frac{Δ {Abs}_{control} - Δ {Abs}_{sample}}{Δ Abscontrol} ) \times 100$$

Statistical Analysis

All experiments are performed twice and analysis for each experiment is carried out in triplicate. Means, standard deviations, the degree of significance (p<0.05 —One way ANOVA and t-test) are determined using Microsoft Excel XP. Inhibition concentration (IC₅₀) values are calculated using ED50plus vol. 1 developed by Vargas

Results and Discussion

Phenolic Content of Maple Syrup Extracts and their Antioxidant Activity

On a dry weight (DW) basis, the MS-EtOAc extract has the highest total phenolic content (340 mg/g DW) followed by the MS-BuOH (30 mg/g DW) extract (Table 1). Similarly, for the antioxidant activity as measured by the DPPH radical scavenging assay, the MS-EtOAc extract exhibits higher antioxidant activity (IC₅₀=77.5 ppm) compared to the MS-BuOH fractions (IC₅₀>1000 ppm) (Table 1).

The HPLC-UV chromatograms of MS-EtOAc and MS-BuOH (shown in FIGS. 4A and B, respectively), reveal the complexity of these extracts with regards to their phenolic constituents. While the isolation and identification of the phenolic constituents from MS-BuOH have been reported (Li & Seeram, 2010), those in the MS-EtOAc are not isolated in the current document. However, previous studies by Kermasha et al. (1995) have shown that when ethyl acetate is used as an extraction solvent of maple syrup, it results in a high recovery of phenolic compounds. This would explain the higher observed antioxidant activity of ethyl acetate compared to butanol extracts (Table 1).

TABLE 1
Total phenolic contents and DPPH free-radical
scavenging activity of phenolic-enriched maple syrup
extracts.

| | Total phenolic | DPPH free radical |
|---|---|---|
| | content | scavenging activity |
| Samples | (mg/g GAE DW | (IC₅₀) (ppm) |
| MS-EtOAc | 340 ± 4.2 | 75.5 |
| MS-BuOH | 30 ± 2.9 | >1000 |

It is previously reported that the ethyl acetate extract of maple syrup contains a wide variety of phenolic phytochemicals including small phenolic molecules and flavonoids, predominantly as flavonols and flavanols (Abou-Zaid et al., 2008; Kermasha et al., 1995). Recently, it has been reported that the butanol extract of maple syrup (MS-BuOH) predominantly contained lignans, coumarins, and a stilbene, along with several previously reported small phenolic compounds (Li & Seeram, 2010). It should be noted that similar to other food matrices, the utilization of different organic solvents for extraction of maple syrup yields extracts with differing phenolic profiles. While both MS-EtOAc and MS-BuOH contain predominantly phenolic compounds, their individual phenolic constituents are quite different as previously reported (Abou-Zaid et al., 2008; Li & Seeram, 2010).

Yeast/Rat α-Glucosidase and Porcine α-Amylase Inhibition Assay

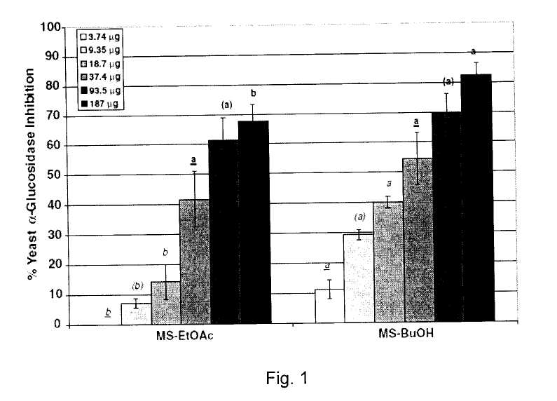

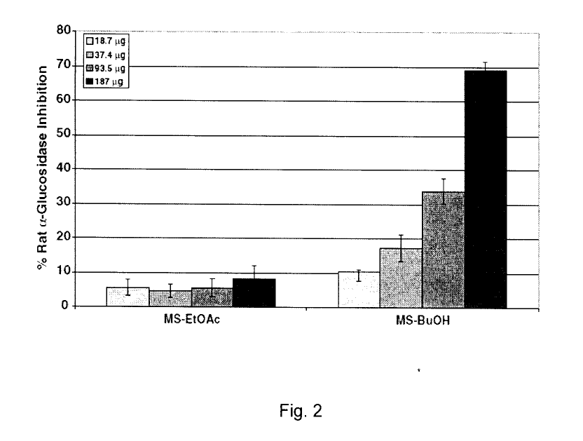

The extracts are standardized to phenolic content of 3.75 mg GAE/mL and assayed for yeast α-glucosidase inhibition. Both extracts have a dose-dependent α-glucosidase inhibitory activity with the MS-BuOH having highest (82% at highest dose, IC₅₀ 68.38 μg phenolics) followed by MS-EtOAc (67% at highest dose, IC₅₀ 107.9 μg phenolics) (FIG. 1). Yeast α-glucosidase assay can be an inexpensive and rapid method to screen for potential α-glucosidase inhibitors as done in the initial assays reported here. However, based on the observed inhibitory activities in the yeast α-glucosidase assay, MS-EtOAc and MS-BuOH are further evaluated for rat α-glucosidase inhibition. The results in the rat α-glucosidase assay show that MS-BuOH extract has a higher dose dependent inhibitory activity than the MS-EtOAC extract (69% at the highest dose, IC₅₀ 135 μg phenolics and 8% at the highest dose, IC₅₀>187 μg phenolics, respectively) (FIG. 2). It is important to note that the MS-EtOAC extract has almost no activity, since no dose-dependency is indicated and the observed results could be due to the limitation of the assay at very low inhibitory activities (FIG. 2).

The above findings indicate that when the extracts are evaluated at equivalent phenolic content, the MS-BuOH exhibited higher α-glucosidase inhibition potential in the yeast based assay (FIG. 1). Similarly, when MS-EtOAC and MS-BuOH are further evaluated for rat α-glucosidase inhibition, it is clear that MS-BuOH fraction had higher potential for α-glucosidase inhibition in the rat-based assay (FIG. 2). These results suggest that the unique combination of phenolic phytochemicals present in the MS-BuOH extract has higher potential for α-glucosidase inhibition (Li & Seeram, 2010). The importance of phenolic profiles and the potential synergies among individual phenolic phytochemicals for α-glucosidase inhibition and other biological effects has been well established (Apostolidis et al., 2006; Kwon et al., 2006; Seeram, Adams, Hardy, & Heber, 2004).

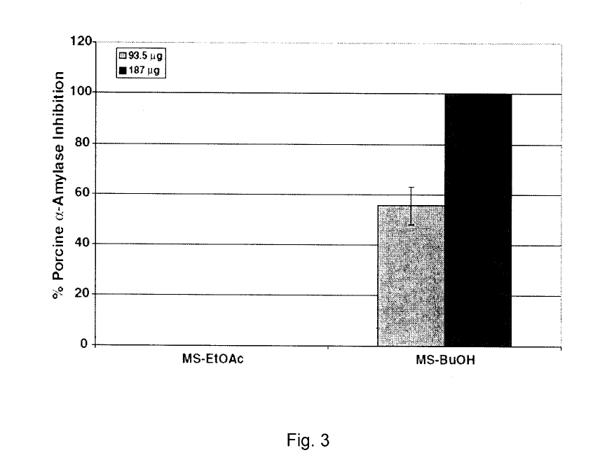

The phenolic standardized MS-BuOH and MS-EtOAc extracts are further assayed for α-amylase inhibition in a porcine based assay. At the test concentrations, the MS-EtOAc extract has no inhibitory activity (IC₅₀>187 μg) while the MS-BuOH extract has α-amylase inhibition with IC₅₀=103 μg phenolics (FIG. 3). Previous reports have indicated that phenolic compounds have lower α-amylase inhibitory activity and a stronger inhibition activity against yeast α-glucosidase (Apostolidis & Lee, 2010; Kwon et al., 2006). The MS-BuOH extract of maple syrup has significantly milder α-amylase inhibitory activity (FIG. 3) compared to its observed yeast α-glucosidase inhibitory activity (FIG. 1), however, it appears to have a rat α-glucosidase inhibitory activity at similar levels (FIG. 2). Optimum inhibition of both α-amylase and α-glucosidase enzymatic activities could result in slower oligosaccharide release from starch, with subsequent slower glucose absorption in the small intestine, thus better moderating postprandial blood glucose increase.

Phenolic compounds are secondary metabolites of plant origin which constitute one of the most abundant and ubiquitous groups of natural metabolites and form an important part of both human and animal diets (Bravo, 1998; Crozier et al., 2000; Vattem, Ghaedian, & Shetty, 2005). Many studies have shown that phenolic phytochemicals have high antioxidant activity and other biological properties (Al-Farsi, Alsalvar, Morris, Baron, & Shahidi, 2005; Seeram et al., 2005; Shahidi & Ho, 2005; Yahia, 2010). Various researchers have identified the phenolic constituents of maple syrup in different extracts (Abou-Zaid et al., 2008; Filipie & Underwood, 1964; Kermasha et al., 1995; Li & Seeram, 2010; Potter & Fagerson, 1992) and are further related to antioxidant (Legault et al., 2010; Li & Seeram, 2010; Theriault et al., 2006; Yosikawa, Kawahara, Arihara, & Hashimoto, 2010) human cancer cell antiproliferative (Legault et al., 2010; Theriault et al., 2006) and anti-inflammatory properties (Legault et al., 2010). The present document show that maple syrup phenolic-enriched extracts have type 2 diabetes management potential, via inhibition of carbohydrate hydrolyzing enzymes, with the MSBuOH fraction having the highest bioactivity. There are therefore potential bioactivities unique to the MS-BuOH phenolic phytochemicals in relation to type 2 diabetes management.

During the production of maple syrup, apart from natural phenolic constituents, other unique phenolic and non-phenolic compounds are formed during the intensive heating involved in transforming sap into syrup (Li & Seeram, 2010). For example, a novel process-derived phenolic compound in MS-BuOH has recently been identified (Li & Seeram, 2011). Thus these process-derived compounds may impart additional biological effects to maple syrup, and therefore contribute to the observed health benefits and biological activities of maple syrup.

### CONCLUSION

The present document is the first report of the type 2 diabetes management potential of maple syrup. These findings indicate that compared to MS-EtOAC, the MS-BuOH is the most active extract and it has a particular phenolic profile and related bioactivities. The understanding of the mechanism of action and identification of compounds responsible for the observed α-glucosidase and α-amylase inhibitory activities coupled with animal and clinical trials could lead to the development of a maple syrup sweetener with lower glycemic index designed for type 2 diabetes prevention.

### Example 2

### In Vitro Phenolic-Mediated Anti-hyperglycemic Properties of Sugar and Red Maple Leaf Extracts

The objective of the current example is to evaluate In Vitro Phenolic-Mediated Anti-hyperglycemic Properties of Sugar and Red Maple Leaf Extracts.

Red maple and sugar maple (Acer rubrum and Acer saccharum, respectively) leaves are collected in the summer and fall of 2010 from Canada and are evaluated for seasonal variation in terms of phenolic contents, antioxidant activities, and α-glucosidase and α-amylase inhibitory activities, relevant to type 2 diabetes management. Dried leaves are extracted in methanol, dried under vacuum and suspended in DMSO. The phenolic contents of summer red maple leaves (RML-S) and summer sugar maple leaves (SML-S) are higher than red and sugar maple leaves collected in the fall (RML-F and SML-F, respectively). The extracts are also assayed for α-glucosidase inhibitory activities with SML-S extracts having the highest inhibitory activity (IC₅₀=21 μg/mL). The α-glucosidase inhibitory activities are dependent on both phenolic content and phenolic profile. When the α-amylase inhibitory activity is evaluated, a non-phenolic dependent seasonal variation is observed only with red maple leaves, with RML-F having the highest inhibitory activity (IC₅₀ 7.3 mg/mL). These results show that sugar and red maple leaf extracts have potential for phenolic-mediated α-glucosidase inhibition, relevant to type 2 diabetes management, with SML-S extract having the highest bioactivity, which could be related to unique phenolic compounds identified in this research.

Introduction

Non-insulin dependent diabetes mellitus, a common disorder of glucose and fat metabolism, is strongly associated with diets high in calories and linked to changes in dietary pattern towards high calorie sweetened foods with disaccharides such as maltose and sucrose (Garg et al. 1994). Worldwide, at least 220 million people have diabetes and this figure is estimated to double by 2030 (World Health Organization 2011). In the United States alone, in 2007, 23.7 million people (10% of American adults) had diabetes and this figure is expected to jump to 33% (i.e. one-third of all American adults) by 2050 (Center for Disease Control 2011).

Hyperglycemia is a condition characterized by a rapid rise in blood glucose levels subsequent to hydrolysis of starch by pancreatic α-amylase and intestinal α-glucosidase-mediated absorption of glucose in the small intestine. One of the therapeutic approaches for decreasing postprandial hyperglycemia is to retard absorption of glucose by the inhibition of carbohydrate hydrolyzing enzymes, α-amylase and α-glucosidase, in the digestive organs (Deshpande et al. 2009). Therefore, inhibition of these enzymes can significantly decrease the postprandial hyperglycemia after a mixed carbohydrate diet and can be a key strategy in the control of diabetes mellitus (Hirsh et al. 1997). Recent research findings have shown that plant-derived phenolics play a role in mediating α-glucosidase and α-amylase inhibition and thus have potential to contribute to the management of type-2 diabetes (Hogan et al. 2010; Apostolidis et al. 2011a; Apostolidis et al. 2011b; Apostolidis and Lee 2010; Kwon et al. 2007).

Maple syrup is a natural sweetener and is the largest commercially available food product that is totally derived from the sap of deciduous trees. It is obtained by concentrating the sap collected from certain maple species including the sugar maple (Acer saccharum Marsh.) and red maple (Acer rubrum L.) trees which are both native to North America (Ball 2007; Van Den Berg and Perkins 2007). Maple syrup is produced primarily in North America with the vast majority of the world's supply coming from Canada (85%; primarily Quebec), followed by United States (Perkins and Van Der Berg 2009). Previous reports have shown that maple syrup contains a wide variety of phenolic phytochemicals (Li and Seeram 2011; Li and Seeram 2010; Abou-Zaid et al. 2008), which may have positive effects on human health. Recently, phenolic-enriched extracts of maple syrup are shown to have antioxidant, anti-mutagenic and human cancer cell anti-proliferative properties (Li and Seeram 2011; Li and Seeram 2010; Legault et al. 2010; Theriault et al. 2006). In addition, Apostolidis et al. (2011a) reported that maple syrup extracts had phenolic-mediated potential for type 2 diabetes management via inhibition of the carbohydrate hydrolyzing enzyme α-glucosidase.

The sugar maple and red maple species are native to Northeastern American forests and their leaves are responsible for most of the red and orange autumn coloration of these forests. The variation in color pigmentation occurs due to changes in three plant pigments among the trees (Schaberg et al. 2008). Two of these classes of pigments, chlorophylls that appear green and carotenoids that appear yellow, are synthesized during the growing season to enable or protect photosynthetic light capture (Schaberg et al. 2008). In contrast, anthocyanin pigments that give leaves a red or purple color are often synthesized toward the end of the leaf's lifespan (Field et al. 2001; Matile 2000). Anthocyanins are a non-functional by-product of leaf senescence (Archetti 2000; Matile 2000). Their biosynthesis is induced due to exposure to a wide variety of stresses such as UV-B radiation (Mendez at al. 1999), osmotic stress (Kaliamoorthy and Rao 1994), drought (Balakumar at al. 1993), low temperatures (Krol et al. 1995), nutrient deficiencies (Rajendran et al. 1992), wounding (Ferreres et al. 1997), pathogen infection (Dixon et al. 1994) and exposure to ozone (Foot et al. 1996). The observed anthocyanin buildup following exposure to stress raises the possibility that anthocyanins may function, in part, to prevent stress-induced damage. The phenolic variation in sugar maple leaves harvested in fall (autumn) and summer is evaluated by Baldwin et al. (1987) and the results showed that during fall, sugar maple leaves have higher phenolic contents when compared to summer.

Although sugar and red maple belong to the same family (Aceraceae) of trees, their leaf phenolic profile has certain differences and similarities. Both red and sugar maple leaves contain small amounts of methyl gallate (Abou-Zaid et al. 2009), while only red maple contains a rare galloyl sugar, galloyl rhamnose (Abou-Zaid and Nozzolillo 1995). In addition, red maple is highly resistant to forest tent caterpillar, in contrast to sugar maple, due to the presence of ethyl-m-digallate at high amount (Abou-Zaid et al. 2001). Recently, Honma et al (2010) reported that Sugar maple leaf extracts had phenolic-mediated potential for type 2 diabetes management via inhibition of the carbohydrate hydrolyzing enzyme α-glucosidase and identified acertanin (ginnalin A) as the active compound. On the other hand, Japanese red maple (Acer pycnanthum K. Koch) appeared to have similar effect against type 2 diabetes, but the active compounds for the observed effect are identified as ginnalins B and C (Honma et al. 2011).

The phenolic polymorphism of red and sugar maple leaves and the potential health benefits that derive from them have significant applications. The aim of this document is to evaluate the differences between sugar and red maple leaves, collected in summer and fall in terms of phenolic contents, antioxidant activities, and type 2 diabetes management via inhibition of the carbohydrate hydrolysis enzymes, α-glucosidase and α-amylase.

Materials and Methods

General Experimental Procedures.

High performance liquid chromatography (HPLC) is performed on a Hitachi Elite LaChrom system consisting of a L2130 pump, L-2200 autosampler, and a L-2455 Diode Array Detector all operated by EZChrom Elite software. All solvents are of either ACS or HPLC grade and are purchased from Wilkem Scientific (Pawtucket, R.I.). α-Amylase (porcine pancreatic, EC 3.2.1.1), α-glucosidase (yeast, EC 3.2.1.20) and rat intestinal powder are purchased from Sigma-Aldrich (St. Louis, Mo.). Unless otherwise specified, all other chemicals of analytical grade are purchased from Sigma-Aldrich.

Sample Preparation and HPLC Phenolic Profiling.

Sugar maple leaves (SML) and red maple leaves (RML) are collected in Canada in 2010 during summer (SML-S and RML-S) and fall (SML-F and RML-F) and are to the laboratory as previously reported (González-Sarrias et al. 2011). Voucher specimens are deposited at the Bioactive Botanical Research Laboratory in the University of Rhode Island, R.I., USA. Leaves are kept frozen in the laboratory until extraction. Air-dried leaves of red maple and sugar maple (8.5 g) are extracted by sonication with methanol (150 mL) at room temperature for 40 min. The methanol extracts are dried under reduced vacuum. The SML and RML leaf extracts (10 mg/mL in DMSO) are injected into HPLC system with a Luna C18 column (250×4.6 mm i.d., 5 μM; Phenomenex) and 15 μL injection volume. A gradient solvent system consisting of solvent A (0.1% aqueous trifluoroacetic acid) and solvent B (methanol, MeOH) is used at a flow rate of 0.75 mL/min as follows: 0-30 min, 10% to 60% B; 30-35 min, 60% to 100% B; 35-40 min, 100% B; 40-41 min, 100% to 10% B; 41-51 min, 100% B. A linear standard curve between ginnalin A concentration and UV absorbance area at 280 nm is constructed for quantification and standardization purposes (r²=0.9942).

Total Phenolic Content.

The total phenolics are determined following the procedure modified from Shetty et al (1995). Briefly, 1 mL extract is transferred into a test tube and mixed with 1 mL 95% ethanol and 5 mL distilled water. To each sample, 0.5 mL 50% (v/v) Folin-Ciocalteu reagent is added and vortex mixed. After 5 min, 1 mL 5% Na₂CO₃ is added to the reaction mixture and allowed to stand for 60 min. The absorbance is read at 725 nm using a Thermo Scientific Genesys 10uv spectrophotometer (Madison, Wis.). The absorbance values are converted to total phenolics and are expressed in mg gallic acid/g sample dry weight (DW). A standard curve is established using varying concentrations of gallic acid in ethanol.

Antioxidant Assay.

The antioxidant potential of the extracts is determined on the basis of the ability to scavenge the 1,1-diphenyl-2-picrylhydrazyl (DPPH) radicals as previously described (Nanjo et al. 1996). The DPPH radical scavenging activity of ascorbic acid (vitamin C) and the synthetic commercial antioxidant, butylated hydroxytoluene (BHT), are also assayed as positive controls. The assay is conducted in a 96-well format using serial dilutions of 100 μL aliquots of test compounds (ranging from 2500 to 26 μg/mL), ascorbic acid (1000-10.4 μg/mL), and BHT (250,000-250 μg/mL). Then DPPH (150 μL) is added to each well to give a final DPPH concentration of 137 μM. Absorbance is read after 30 min at 515 nm, and the scavenging capacity (SC) is calculated as SC %=(A₀−A₁/A₀)×100, where A₀ is the absorbance of the reagent blank and A₁ is the absorbance of test samples. All tests are performed in triplicate. IC₅₀ value denotes the concentration of sample required to scavenge 50% of DPPH radicals.

Yeast α-glucosidase Inhibition Assay.

A mixture of 50 μL of extract and 100 μl of 0.1 M phosphate buffer (pH 6.9) containing yeast α-glucosidase solution (1.0 U/ml) is incubated in 96 well plates at 25° C. for 10 min. After pre-incubation, 50 μl of 5 mM p-nitrophenyl-α-D-glucopyranoside solution in 0.1M phosphate buffer (pH 6.9) is added to each well at timed intervals. The reaction mixtures are incubated at 25° C. for 5 min. Before and after incubation, absorbance is recorded at 405 nm by a micro-plate reader (VMax, Molecular Device Co., Sunnyvale, Calif., USA) and compared to that of the control which had 50 μL buffer solution in place of the extract. The α-glucosidase inhibitory activity is expressed as % inhibition and is calculated as follows:

$$\% Inhibition = ( \frac{Δ {Abs}_{control} - Δ {Abs}_{sample}}{Δ {Abs}_{control}} ) \times 100$$

Rat α-glucosidase Inhibition Assay.

The rat intestinal α-glucosidase assay is conducted according to the method of Kwon et al (2007) with a slight modification. A total of 1 g of rat-intestinal acetone powder is suspended in 10 mL of 0.9% saline, and the suspension is sonicated twelve times for 30 sec at 4° C. After centrifugation (10000×g, 30 min, 4° C.), the resulting supernatant is used for the assay. Sample solution (50 μL) and 0.1 M phosphate buffer (pH 6.9, 100 μL) containing α-glucosidase solution is incubated at 25° C. for 10 min. After preincubation, 5 mM p-nitrophenyl-α-D-glucopyranoside solution (50 μL) in 0.1M phosphate buffer (pH 6.9) using a multi-channel pipette. The reaction mixtures are incubated at 25° C. Before and after incubation, absorbance is read at 405 nm and compared to a control which had 50 μL of buffer solution in place of the extract by micro-plate reader. The α-glucosidase inhibitory activity is expressed as % inhibition and is calculated as follows:

$$\% Inhibition = ( \frac{Δ {Abs}_{control} - Δ {Abs}_{sample}}{Δ {Abs}_{control}} ) \times 100$$

Porcine α-amylase Inhibition Assay.

A mixture of 50 μL of extract and 50 μL 0.02 M sodium phosphate buffer (pH 6.9 with 0.006 M sodium chloride) containing α-amylase solution (13 U/mL) is incubated at 25° C. for 10 min using an 1.5 mL Eppendorf tube. After pre-incubation, 50 μL of 1% soluble starch solution in 0.02 M sodium phosphate buffer (pH 6.9 with 0.006 M NaCl) is added to each well at timed intervals. The reaction mixtures are then incubated at 25° C. for 10 min followed by addition of 1 mL dinitrosalicylic acid color reagent. The test tubes are then placed in a boiling water bath for 10 min to stop the reaction and cooled to room temperature. The reaction mixture is then diluted with 1 mL distilled water and absorbance is read at 540 nm using a 96-well mircoplate reader.

$$\% Inhibition = ( \frac{Δ {Abs}_{control} - Δ {Abs}_{sample}}{Δ {Abs}_{control}} ) \times 100$$

Statistical Analyses.

All experiments are performed twice and analyses for each experiment are carried out in triplicate. Means, standard deviations, the degree of significance (p<0.05—One Way ANOVA and t-Test) are determined using Microsoft Excel XP. Inhibition concentration (IC₅₀) is calculated using ED50plus vol. 1 developed by Vargas

Results and Discussion

Total Phenolic Content, Phenolic Profile and Antioxidant Activity.

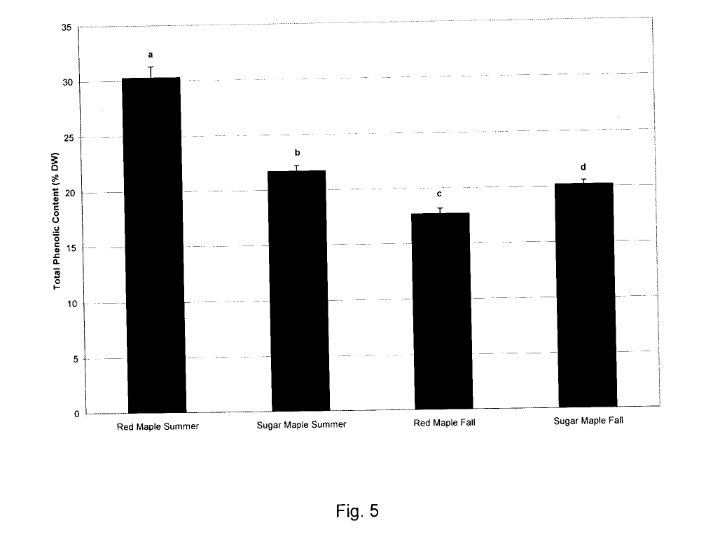

The total phenolic contents in SML and RML collected in summer and fall are evaluated. Seasonal variation between leaves collected in the summer and fall are observed both in SML and RML (FIG. 5). More specifically, RML-S yielded a total phenolic content of 30% dry weight (DW), which is significantly higher than RML-F (17% DW) (FIG. 5). When the total phenolic content in SML is evaluated, it is noted that SML-S had a higher total phenolic content (22% DW) than SML-F (20% DW) (FIG. 5).

Previous reports have shown that fall (autumn) sugar maple leaves have higher phenolic contents than summer (Baldwin et al. 1987). In addition, phenolic biosynthesis has been shown to depend on a variety of environmental factors such as UV-B radiation (Mendez et al. 1999), osmotic stress (Kaliamoorthy and Rao 1994), drought (Balakumar et al. 1993), low temperatures (Krol et al. 1995), nutrient deficiencies (Rajendran et al. 1992), wounding (Ferreres et al. 1997), pathogen infection (Dixon et al. 1994) and exposure to ozone (Foot et al. 1996). The present results show that SML and RML are most probably under certain stress during the summer months, which significantly affects their phenolic biosynthesis. After a brief temperature survey for the temperature fluctuation in the summer and fall months in Canada for years 2007-2010 (Weather Underground 2011), it is noted that in July 2010 a high mean temperature is observed (81° F. compared to 76°, 76° and 73° F. observed for 2007, 2008 and 2009 respectively). This discrepancy could be a factor contributing to the higher phenolic content observed in the summer specimens (FIG. 5).

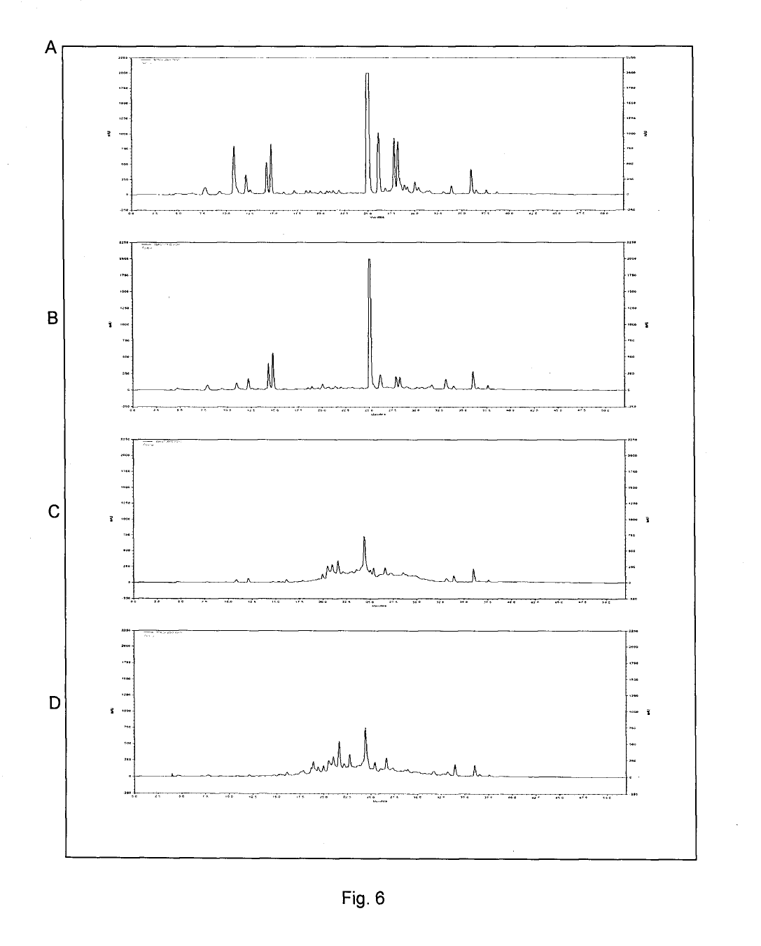

The phenolic profiles of the tested extracts are obtained by determining the phenolic constituents of SML and RML and a seasonal effect on the observed compounds are also studied (FIG. 6). The chromatograms show that there is a difference in the phenolic profile between SML and RML (FIG. 6). The major phenolic compounds identified in RML are ginnalin A (25 min) and ginnalins B & C (14-15 min) as previously reported (González-Sarrias et al. 2011). In SML, ginnalin A is identified, while a group of unknown compounds eluted between 18-23 min (FIG. 6). When the RML-S and RML-F chromatograms are compared, it is noted that the profiles are very similar. However, in SML-S extracts a few peaks observed in SML-F between 18-20 min do not exist, but 2 peaks (absent in SML-F) between 10.5-12.5 min appear (that are absent in SML-F) (FIG. 6). In addition, small peaks before and after ginnalin A are absent in SML-F but appear in SML-S (FIG. 2). These observations suggest that there are differences in the phenolic profile between RML and SML, as well as between SML-S and SML-F.

The antioxidant activity is evaluated based on the DPPH free-radical scavenging activity. The results show that both RML and SML summer leaves have higher antioxidant activities than the fall leaves (Table 1). More specifically, RML-S has an IC₅₀ of 8.5 ppm while the IC₅₀ for RML-F is 15.3 ppm (Table 2), while SML-S has a higher antioxidant activity than SML-F (15 and 19.1 ppm, respectively) (Table 2).

TABLE 2
IC₅₀ values for DPPH free-radical scavenging activity of sugar and
red maple leaves collected in summer and fall.

| Samples | IC₅₀ (ppm) |
|---|---|
| Red Maple Leaves—Summer (RML-S) | 8.53 |
| Sugar Maple Leaves—Summer (SML-S) | 15.0 |
| Red Maple Leaves—Fall (RML-F) | 15.3 |
| Sugar Maple Leaves—Fall (SML-F) | 19.1 |

These results correlate with the observed total phenolic contents since the summer months in both SML and RML have both higher phenolic contents and DPPH free-radical scavenging activities.

Yeast and Rat Intestine α-glucosidase Inhibition.

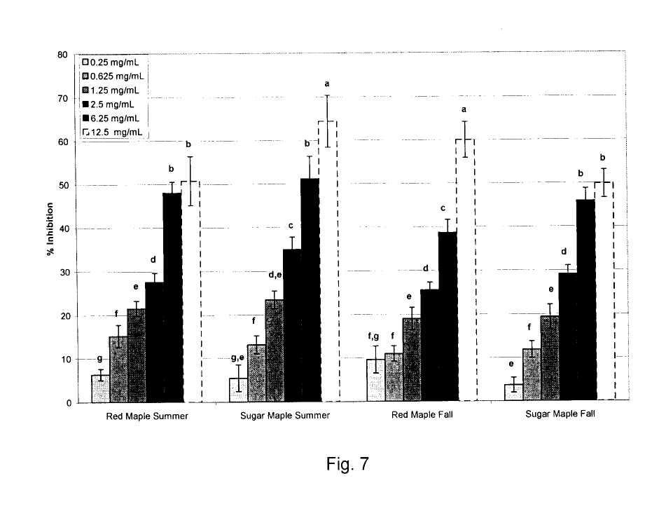

The α-glucosidase inhibitory activities of the collected samples are evaluated using both yeast and rat intestinal enzyme sources. Due to absorbance reading being interfered by the dark color of the samples, the maximum concentration tested in this assay is 12.5 mg/mL. At the tested doses, the observed inhibitory activities are not sufficient to give an accurate IC₅₀ value (FIG. 7). These dose-dependent observations indicate that SML-S has the highest inhibitory potential, among all samples tested, while all other samples show similar activities (FIG. 7).

Based on previous observations, natural compounds tend to have higher yeast α-glucosidase inhibitory activities, than the rat intestinal α-glucosidase activities (Apostolidis et al. 2011a). In order to observe a better dose-dependent inhibitory effect and estimate the accurate IC₅₀ values, the yeast α-glucosidase inhibitory activities of the samples are determined (Table 3). Similarly to rat α-glucosidase inhibition results, SML-S has the highest inhibitory potential (IC₅₀ 21 μg/mL). In addition, both summer SML and RML samples have higher inhibitory activities than the fall samples (IC₅₀: SML-F-39 μg/mL, RML-S-115 μg/mL, RML-F-128 μg/mL) (Table 3).

TABLE 3
IC₅₀ values for yeast α-glucosidase and porcine α-amylase inhibitory
activities of sugar and red maple leaves collected in summer and fall.

| | Yeast α-Glucosidase | Porcine α-Amylase |
|---|---|---|
| Samples | IC₅₀ (μg/mL) | IC₅₀ (mg/mL) |
| Red Maple Leaves— | 115 | 8.4 |
| Summer (RML-S) | | |
| Sugar Maple Leaves— | 21.4 | 10.4 |
| Summer (SML-S) | | |
| Red Maple Leaves—Fall | 128 | 7.3 |
| (RML-F) | | |
| Sugar Maple Leaves— | 38.8 | 10.8 |
| Fall (SML-F) | | |

In terms of seasonal variation all summer samples have higher phenolic contents (FIG. 5) and yeast α-glucosidase inhibitory activities (Table 3). Among samples, RML-S has the highest phenolic content, while SML-S and SML-F has higher α-glucosidase inhibitory activities (FIG. 7 and Table 3). Previously, it is shown that ginnalins A, B and C have α-glucosidase inhibitory activity (Honma et al. 2011; Honam et al. 2010). However, these observations indicate that a group of unknown phenolic compounds that are present only in SML and eluted between 18-23 min, has a more pronounced effect on α-glucosidase inhibition. The observed high α-glucosidase inhibitory activities could be due to the synergistic effects of the different phenolic compounds detected in SML and deserves further investigation. The importance of phenolic profiles and the potential synergies among individual phenolic phytochemicals for α-glucosidase activities and other biological effects has been well established (Apostolidis et al. 2006; Seeram et al. 2004).

Porcine α-amylase Inhibition.

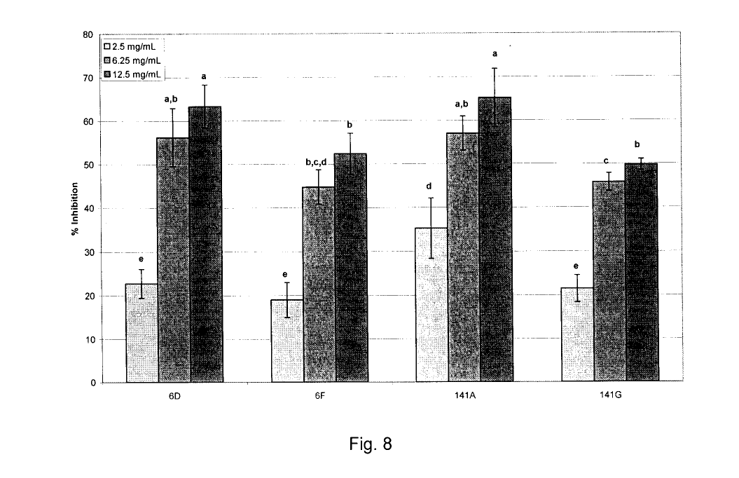

The effect of the extracts on the inhibition of porcine α-amylase are evaluated and all samples appear to have a dose-dependent inhibitory activity. More specifically, no significant difference is observed between SML-S and SML-F (IC₅₀ 10.4 and 10.8 mg/mL, respectively) (FIG. 8 and Table 2). On the other hand, RML-F has a higher α-amylase inhibitory activity than RML-S (IC₅₀ 8.4 and 7.3 mg/mL, respectively) (FIG. 8 and Table 3).

The observed results show that there is seasonal variation in α-amylase inhibitory activity only in RML and that this variation is not phenolic-dependent (FIG. 5, FIG. 8, Table 3). Also, it should be noted that SML-S extracts, that have the highest α-glucosidase inhibitory activity (Table 2 and FIG. 3), appear to have milder α-amylase inhibition (Table 3 and FIG. 8). Previous reports have indicated that phenolic compounds have a low α-amylase inhibitory activity, but a strong inhibitory activity against yeast α-glucosidase (Apostolidis et al. 2007a; Apostolidis and Lee 2010; Kwon et al. 2007). It is important to point out that acarbose is a chemical drug specifically designed for α-glucosidase inhibition and has various side effects which include abdominal distention, flatulence, meteorism and possibly diarrhea (Bischoff et al. 1985). It has been suggested that such side effects are caused by the excessive inhibition of pancreatic α-amylase resulting in the abnormal bacterial fermentation of undigested carbohydrates in the colon (Horii et al. 1987; Bischoff et al. 1985). Optimum inhibition of both α-amylase and α-glucosidase activities could result in slower oligosaccharide release from starch, with subsequent slower glucose absorption in the small intestine, thus better moderating postprandial blood glucose increase.

Conclusions

The present document reports the seasonal variation in sugar and red maple leaves harvested in summer and fall, in terms of total phenolic contents and corresponding antioxidant and carbohydrate hydrolyzing enzyme inhibitory activities. Based on the above-mentioned observations, sugar maple leaves collected in the summer of 2010 have superior potential for α-glucosidase inhibition, relevant to type 2 diabetes management. Additionally, this effect is dependent on both the phenolic contents and the individual phenolic profiles. The understanding of the mechanism of action and identification of compounds responsible for the observed α-glucosidase and α-amylase inhibitory activities coupled with animal and clinical trials could lead to the development of maple tree leaf ingredients designed for type-2 diabetes prevention.

### Example 3

### Sugar and Red-Leaf Maple Tree Extracts Inhibit Growth of Human Tumorigenic but not Non-Tumorigenic Colon Cells Mediated Through Cell Cycle Arrest

### Methods and Materials

General Experimental Procedures

Nuclear Magnetic Resonance (NMR) spectra for all compounds are recorded on a Bruker 400 MHz Biospin spectrometer (¹H: 400 MHz, ¹³C: 100 MHz) using deuterated methanol (methanol-d₄) as solvent. Mass Spectral (MS) data are carried out on a Q-Star Elite (Applied Biosystems MDS) mass spectrometer equipped with a Turbo Ionspray source and are obtained by direct infusion of pure compounds. High performance liquid chromatography (HPLC) are performed on a Hitachi Elite LaChrom system consisting of a L2130 pump, L-2200 autosampler, and a L-2455 Diode Array Detector all operated by EZChrom Elite software. All solvents are either ACS or HPLC grade and are obtained from through Wilkem Scientific (Pawcatuck, R.I.). Unless otherwise stated, all reagents including the MTS salt [3-(4,5-dimethylthiazol-2-yl)-5-(3-carboxymethoxyphenyl)-2-(4-sulfenyl)-2H-tetrazolium salt], gallic acid, Folin-Ciocalteu reagent and etoposide standards are obtained from Sigma-Aldrich.

Plant Materials

Plant materials are collected in summer of 2009 by the Federation of Maple Syrup Producers of Quebec (Quebec, Canada), shipped to our laboratory in August 2009, and identified by Mr. J. Peter Morgan (Senior Gardener, College of Pharmacy, University of Rhode Island). Voucher specimens for all plant materials are assigned unique identification codes and are deposited in the Heber Youngken Medicinal Garden and Greenhouse (College of Pharmacy, University of Rhode Island). Voucher specimen codes for the Sugar maple tree parts are: leaves JPMCL1; twigs/stem, JPMCS1; bark JPMCB1; sapwood/heartwood, JPMCH1, and fruit JPMCF2. Voucher specimen codes for the Red-leaf maple tree parts are: leaves, JPMCL2; stem/twigs, JPMCS2; bark JPMCB2; sapwood/heartwood JPMCH2 and fruit JPMCF2.

Preparation of Extracts

Briefly, all plant extracts are prepared using dried and pulverized parts of harvested plants. For each dried and ground maple plant material (ca. 10.0 g), extractions are performed using methanol (3×100 ml) to afford a dried methanol extract, after solvent removal in vacuo. The dried weights of the extracts obtained from the Sugar and Red-leaf maple species are: leaves=0.7 g and 3.3 g; twigs/stem=0.3 g and 0.69 g, bark=0.85 g and 0.80 g; sapwood/heartwood=0.05 g and 0.13 g, fruit 0.6 g and 1.8 g respectively.

Determination of Total Phenolic Content

The total phenolic contents of the maple extracts are determined according to the Folin-Ciocalteu method and is measured as gallic acid equivalents (GAEs) as previously reported by our laboratory (Li et al., 2009). Briefly, the extracts are diluted 1:100, or as appropriate, with methanol/H₂O (1:1, v/v), and 200 μl of sample is incubated with 3 ml of methanol/H₂O (1:1, v/v) and 200 μl of Folin-Ciocalteau reagent for 10 min at 25° C. After this, 600 μl of 20% Na₂CO₃ solution is added to each tube and vortexed. Tubes are further incubated for 20 min at 40° C. After incubation, samples are immediately cooled in an ice bath to room temperature. Samples and standards (gallic acid) are processed identically. The absorbance is determined at 755 nm, and final results are calculated from the standard curve obtained from a Spectramax plate reader.

Analytical HPL-UV Analyses of the Maple Extracts

A Luna C18 column (250×4.6 mm i.d., 5 μM; Phenomenex) with a flow rate at 0.75 ml/min and injection volume of 20 μl for all samples (extracts and ginnalin-A) is used. A gradient solvent system consisting of solvent A (0.1% aqueous trifluoroacetic acid) and solvent B (methanol, MeOH) is used as follows: 0-10 min, from 10 to 15% B; 10-20 min, 15% B; 20-40 min, from 15 to 30% B; 40-55 min, from 30 to 35% B; 55-65 min, 35% B; 65-85 min, from 35 to 60% B; 85-90 min, from 60 to 100% B; 90-93 min, 100% B; 93-94 min, from 100 to 10% B; 94-104 min, 10% B. FIG. 2 shows the HPLC-UV profiles of the maple plant part extracts from the Red-leaf maple (FIG. 2A) and Sugar maple (FIG. 2B) trees, respectively.

HPLC-UV Standardization of Maple Extracts to Ginnalin-A Content A stock solution of 1 mg/ml of a pure standard of ginnalin A (isolated as described below) is prepared in DMSO and then serially diluted to afford samples of 0.5, 0.25, 0.125, 0.0625, 0.03125 mg/ml concentrations, respectively. Each sample is injected in triplicate and a linear six-point calibration curve (r²=0.9997) is constructed by plotting the mean peak area percentage against concentration. Plant extracts are prepared at stock solutions of 2.2 mg/ml in DMSO. All HPLC-UV analyses are carried out with 20 μl injection volumes on a Luna C18 column (250×4.6 mm i.d., 5 μM; Phenomenex) and monitored at a wavelength of 280 nm. A gradient solvent system consisting of solvent A (0.1% aqueous trifluoroacetic acid) and solvent B (methanol, MeOH) is used with a flow rate at 0.75 ml/min as follows: 0-30 min, 10% to 60% B; 30-35 min, 60% to 100% B; 35-40 min, 100% B; 40-41 min, 100% to 10% B; 41-51 min, 100% B. The ginnalin-A concentrations of the maple extracts are quantified based on the standard curve.

Isolation and Identification of Ginnalins A, B and C.

Air-dried and ground twigs/stems (547 g) of the Red-leaf maple species are extracted with methanol (700 ml×3) at room temperature to yield 37 g of dried extract after solvent removal in vacuo. A portion of the dried methanol extract (35 g) is reconstituted in water and subjected to liquid-liquid partitioning sequentially with varying solvents, hexane (500 ml×3), ethyl acetate (500 ml×3) and butanol (500 ml×3). The combined butanol extract, after solvent removal in vacuo, yielded 16.1 g of dried extract. A portion of the dried butanol extract (4 g) is chromatographed on a Sephadex-LH-20 column (4.5×64 cm), eluting with a gradient system of methanol/water (7/3 v/v to 100/0 v/v), and then with acetone/water (7/3 v/v). On the basis of analytical HPLC-UV profiles, fourteen combined fractions (Fr. 1-14) are obtained. Ginnalin-A (also known as acertannin, aceritannin, or 2,6-di-O-galloyl-1,5-anhydro-D-glucitol) (1, 306 mg, brown solid) is obtained from Fr. 5 and identified by NMR (¹H and ¹³C) and mass spectral data which corresponded with literature reports (Song et al., 1982; Honma et al., 2010). Similarly Fr. 2 (1.55 g), which contained a mixture of ginnalins B and C is further purified by semipreparative HPLC-UV. Briefly a portion of Fr. 2 (60 mg) is purified on a Waters Sunfire Prep C18 column (250×19 mm i.d., 5 μm) with a gradient solvent system of MeOH/H₂O and flow rate of 2 ml/min. Both ginnalin-B (2, 17 mg, brown solid) and ginnalin-C (3, 15.7 mg, brown solid) are identified by their by ¹H and ¹³C-NMR data which corresponded with literature (Song et al., 1982).

Cell Lines and Culture Conditions

The extracts are solubilized in DMSO and normalized based on their phenolic content to evaluate their antiproliferative activities against the colon cell lines. Human colon cancer cell lines Caco-2 (adenocarcinoma), HT-29 (adenocarcinoma) and HCT-116 (carcinoma) and the normal colon cells CCD-18Co are obtained from American Type Culture Collection (Rockville, USA). Caco-2 cells are grown in EMEM medium supplemented with 10% v/v fetal bovine serum, 1% v/v nonessential amino acids, 1% v/v L-glutamine and 1% v/v antibiotic solution (Sigma). HT-29 and HCT-116 cells are grown in McCoy's 5a medium supplemented with 10% v/v fetal bovine serum, 1% v/v nonessential amino acids, 2% v/v HEPES and 1% v/v antibiotic solution. CCD-18Co cells are grown in EMEM medium supplemented with 10% v/v fetal bovine serum, 1% v/v nonessential amino acids, 1% v/v L-glutamine and 1% v/v antibiotic solution and are used from PDL between 26 to 35 for all experiments. Cells are maintained at 37° C. in an incubator under a 5% CO₂/95% air atmosphere at constant humidity. The pH of the culture medium is determined using pH indicator paper (pHydrion™ Brilliant, pH 5.5-9.0, Micro Essential Laboratory, NY, USA) inside the incubator. Cells are counted using a hemacytometer and are plated at 3,000-5,000 cells per well, in a 96-well format for 24 or 48 h prior to sample treatment depending on the cell line. All of the test samples are solubilized in DMSO (<0.5% in the culture medium) by sonication and are filter sterilised (0.2 μm) prior to addition to the culture media. Control cells are also run in parallel and subjected to the same changes in medium with 0.5% DMSO.

Cell Proliferation and Viability Tests (Trypan Blue Exclusion and MTS Assays)

At the end of either 48 or 72 h of sample treatment, trypsinised cells (2.5 g/l trypsin, 0.2 g/l EDTA) are suspended in cell culture medium, counted using a Neubauer haemacytometer (Bad Mergentheim, Germany) and viability measured using Trypan blue dye exclusion. Results of proliferation and viability in extract-treated cells are expressed as percentage of those values obtained for control (0.5% DMSO) cells. All experiments are performed in triplicate.

The MTS assay is carried out as described previously (Li et al., 2009) with modifications. At the end of 48 or 72 h of treatment with serially diluted test samples, 20 μl of the MTS reagent, in combination with the electron coupling agent, phenazine methosulfate, is added to the wells and cells are incubated at 37° C. in a humidified incubator for 3 h. Absorbance at 490 nm (OD₄₉₀) is monitored with a spectrophotometer (SpectraMax M2, Molecular Devices Corp., operated by SoftmaxPro v.4.6 software, Sunnyvale, Calif., USA), to obtain the number of cells relative to control populations. 20 μl of etoposide 4 mg/ml (Sigma) is assayed as a negative control of proliferation. The results are expressed as the concentration that inhibit growth of cell by 50% vs. control cells (control medium used as negative control), IC₅₀. Data are presented as the mean±S.D. of three separated experiments on each cell line. Etoposide provided consistent IC₅₀ values of 10-20 μM (HT29, HCT116 and Caco-2) and 30-40 μM for the CCD-18Co cells.

Flow Cytometry Analysis of Cell Cycle

Cells (2×10⁵) are collected after the corresponding experimental periods, fixed in ice-cold ethanol:PBS (70:30) for 30 min at 4° C., further resuspended in PBS with 100 μg/ml RNAse and 40 μg/ml propidium iodide, and incubated at 37° C. for 30 min. DNA content (10,000 cells) is analysed using a FACS Calibur instrument equipped with FACStation running FACS Calibur software (BD Biosciences, San Diego, Calif., USA). The analyses of cell cycle distribution are performed in triplicate for each treatment. The coefficient of variation, according to the ModFit LT Version 2 acquisition software package (Verity Software House, Topsham, Me., USA), is always less than 5%.

Morphological Evaluation of Apoptosis

Cells (2.5×10⁴/ml) are treated for 48 and 72 h and fixed with methanol: acetic acid (3:1, v/v) and stained with 50 mg/ml Hoechst 33242 dye at 37° C. for 20 min. Afterwards, the cells are examined under a Nikon Eclipse TE2000-E inverted microscope (Nikon, N.Y., USA). Etoposide (Sigma) 20 μM is assayed as a standard inducer of apoptosis. Morphological evaluation of apoptosis is carried twice for each sample.

Statistical Analysis

Two-tailed unpaired student's t-test is used for statistical analysis of the data. A p value <0.05 is considered significant.

Results

Standardization of Maple Plant Part Extracts

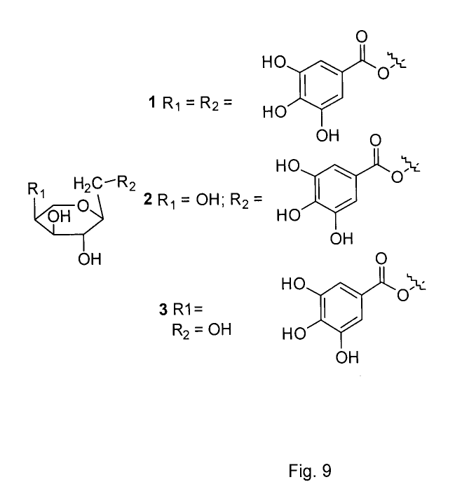

In the current document, various plant parts of the two maple species are subjected to established extraction protocols to enrich them in phenolic contents (Li et al. 2009). The total phenolic content of all of the extracts are evaluated by the Folin-Ciocalteu method and is measured as gallic acid equivalents (GAEs) which ranged from 28.65 to 63.73 mg/L (Table 4). The extracts are further standardized to ginnalin-A (1), ginnalin-B (2) and ginnalin-C (3) contents (chemical structures shown in FIG. 9). These phenolic compounds have previously been isolated from Acer (maple) species (Honma et al., 2010; Song et al., 1982).

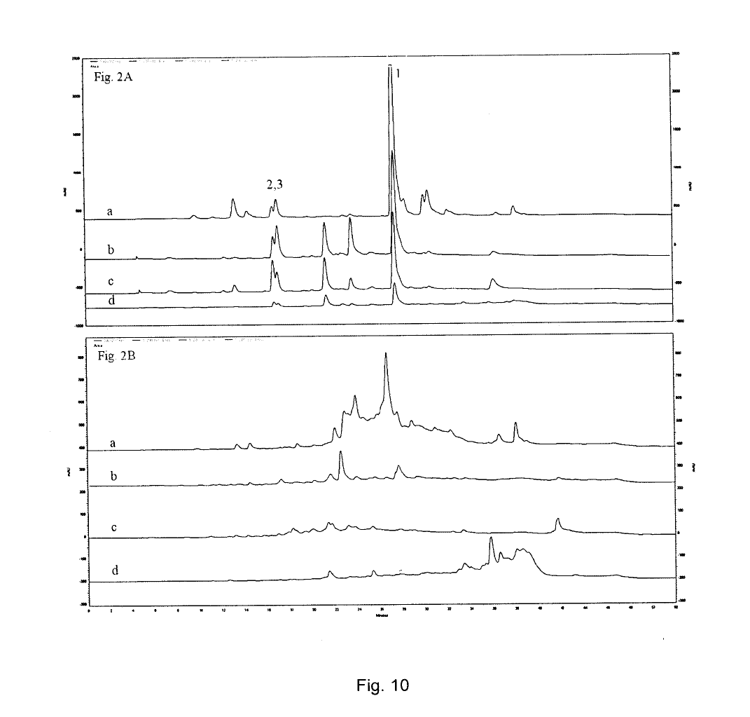

The HPLC-UV chromatograms of the extracts from the different plant parts of the Red-leaf maple and Sugar maple are shown in FIGS. 10A and 10B, respectively. Peaks 1, 2, and 3 correspond to ginnalins-A, B, and C, respectively. Due to the similarity in chemical structures of ginnalins-B and C, it is not surprising to observe that peaks 2 and 3 co-eluted in the HPLC chromatogram. Among these compounds, ginnalin A is the predominant constituent present in the maple extracts. Among the extracts, the leaf extract from the Red-leaf maple species (which is the most active extract in the antiproliferative assay) contained the highest level of ginnalin-A of 45% by weight. On the contrary, the leaf extract of the Sugar maple species contained lower quantities of ginnalin A, estimated from the standard curve to be <3% by weight. The twigs/stem of the Red-leaf maple tree contained the second highest level of ginnalin-A of 24.9% by weight.

Antiproliferative Activity on Cancer Colon Cells by Extracts

The extracts are normalized to deliver equivalent amount of phenolics (50% dry weight) in the antiproliferative assays. All of the maple extracts inhibited the proliferation of HCT-116, Caco-2 and HT-29 cell lines in a time-dependent manner but did not have the similar effect on the normal colon CCD-18Co cells (Table 5). Overall, among the extracts, the leaves and stem extracts showed greater effects than the bark, fruit and sapwood extracts. Also, between the two maple species, extracts of the Red-leaf maple tree showed greater antiproliferative activity than from the Sugar maple tree. In all cases, cell viability is always above 90% at tested doses so extracts are not cytotoxic (results not shown).

After 72 h, the highest antiproliferative effects against the colon cancer cell lines are observed from the leaves and stem extracts of the Red-leaf maple with IC₅₀ values ranging from 35-91 μg/ml and from 55-111 μg/ml, respectively. On the other hand, the IC₅₀ values after treatment with the bark extracts from the Red and Sugar maple tree ranged from 52-91 and from 59-92 μg/ml, respectively. Moderate activity is found in the leaves and stem extracts from the Sugar maple tree (IC₅₀=87-134 and 101-146 μg/I, respectively). Finally, extracts from heartwood and fruits of both species of maple tree showed IC₅₀ values ranging from 127-183) (Table 5).

Among the colon cancer cells, the HCT-116 cells are most sensitive to all of the maple extract treatments compared to the Caco-2 and HT-29 cell lines (Table 5). There is a significant difference between the IC₅₀ values of the extracts against the colon cancer cells compared to the CCD-18Co normal cells (over 2-fold). These results indicate a possible selectivity of the extracts towards colon cancer cells suggesting that these extracts may have potential as colon cancer chemopreventive agents. However further studies would be required to confirm this.

Antiproliferative Activity on Cancer Colon Cells by Ginnalins

Table 6 shows the antiproliferative activities of ginnalins-A, B and C on the colon cancer and normal colon cells. Among the three purified compounds, ginnalin A showed the best activity with IC₅₀ values ranging from 16-24 μg/ml. Among the cell lines, the HCT-116 colon cancer cells are most sensitive to this compound. All ginnalins showed selective activity towards the colon cancer cells than the normal colon cells similar to the maple extracts.

Cell Cycle Distribution Analysis

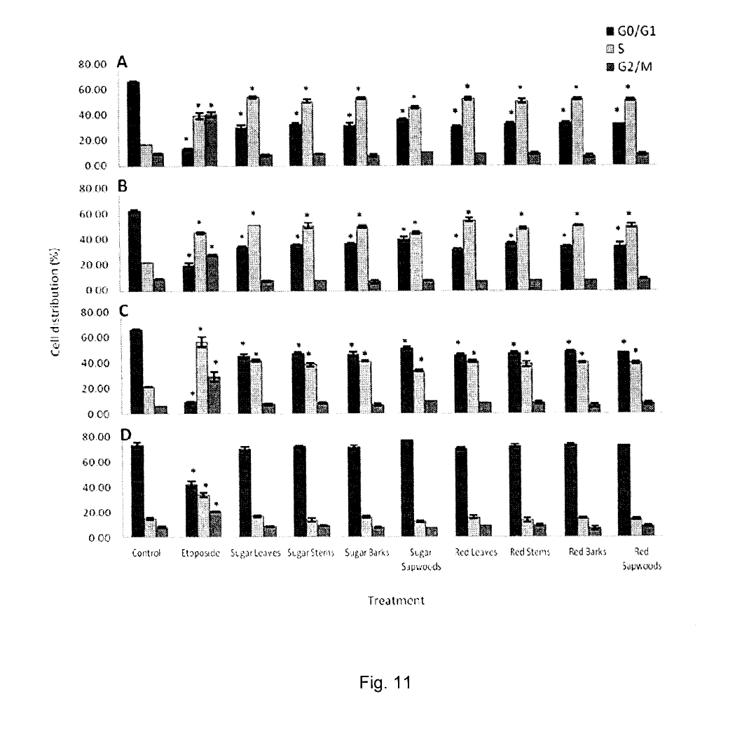

Inhibition of proliferation is further examined by measuring cell cycle distribution. At 48 h of the experiment, HCT-116, Caco-2 and HT-29 control cells are distributed as follows: 58.7±3.6% in G0/G1 phase, 30.8±1.7% in S phase and 10.5±2.0% in G2/M phase; 56.2±2.1% in G0/G1 phase, 31.0±2.4% in S phase and 12.8±0.40% in G2/M phase; and 59.0±1.1% in G0/G1 phase, 31.1±0.9% in S phase and 9.9±0.5% in G2/M phase, respectively (data not shown). At 72 h of the experiment, the proportion of these control cells in the G0/G1 phase increased to 66.3-70.9% whereas cells in the S and G2/M phases decreased to 18.2-23.2% and to 7.2-9.7%, respectively (FIG. 11A-C), indicating that there are no detectable effects of each cell line on cell cycle distribution.

At 48 h treatment with the maple extracts (at doses corresponding to their IC₅₀ values) an increase of cells in S phase (p<0.05) concomitant with a decrease in G₀/G₁ (p<0.05) and a slight increase in G₂/M phase are observed. In accordance with the HCT116 cells being most sensitive among the cell lines in terms of reduced cell growth, changes observed in cell cycle distribution are more pronounced in these HCT-116 cells, with a clear arrest in the S-phase with a range of 45.8-55% (p<0.05). This increase is maintained during the 72 h of sample treatment to 48.6-57.3% (p<0.05), a 150% increase when compared to control cells, in the S phase accompanied by a decrease of cells in G0/G1 phase (range 34.6-42.2%) (p<0.05) whereas no significant changes of the G2/M ratio are observed (FIG. 11A). A similar trend is observed in the Caco-2 and HT-29 colon cancer cells treated with the maple extracts with 84 and 118% increase, and a 72 and 96% increase, in the S arrest at 48 and 72 h, respectively (FIGS. 11B and 11C).

It should be noted that incubation of the normal colon CCD-18Co cells with the various maple plant part extracts for 48 and 72 h did not cause significant changes in cell cycle when compared with control cells (69.3±1.1% in G0/G1 phase, 17.6±0.9% in S phase and 13.1±1.0% in G2/M phase; 76.5±2.0% in G0/G1 phase, 15.2±0.9% in S phase and 8.3±1.1% in G2/M phase, respectively), except with the incubation of etoposide (50 μM) used as a positive control (FIG. 11D).

These results indicate that the compounds present in the maple tree extracts, at subtoxic levels, can inhibit the proliferation of colon cancer cells by blocking the progression of cell cycle at S-phase.

Apoptosis Assessment

Another possible mechanism related to the antiproliferative activity derived from the maple plant part extracts in the colon cancer cells could be mediated by the induction of apoptosis. Therefore, we carried out the morphological evaluation of apoptosis by monitoring for changes in nuclear chromatin distribution that can be stained by the DNA-binding fluorochrome Hoechst 33242 dye. Incubation of the colon cancer cells and normal colon cells with extracts mirrored the pattern followed by untreated cells, thus indicating the absence of apoptosis (data not shown).

TABLE 4
Total polyphenols content (mg/l) and percentage of maple tree
extracts estimated by Folin-Ciocalteu method in 125 mg/l of each sample.

| Source | mg/l | % |
|---|---|---|
| Red-leaf maple leaves | 56.63 | 45.30 |
| Red-leaf maple stems | 63.73 | 50.98 |
| Red-leaf maple barks | 40.30 | 32.24 |
| Red-leaf maple sapwoods | 32.40 | 25.92 |
| Red-leaf maple fruit | 36.36 | 29.08 |
| Sugar maple leaves | 43.79 | 35.04 |
| Sugar maple stems | 54.57 | 43.65 |
| Sugar maple barks | 41.06 | 32.85 |
| Sugar maple sapwoods | 32.24 | 25.79 |
| Sugar maple fruit | 28.65 | 22.92 |

TABLE 5
Antiproliferative data for extracts against human colon cell lines after
48 and 72 h treatment

| | HCT-116 | | HT-29 | |
|---|---|---|---|---|
| | 48 h | 72h | 48 h | 72h |
| Source | IC₅₀ ^{a} | IC₅₀ ^{a} | IC₅₀ ^{a} | IC₅₀ ^{a} |
| Sugar maple leaves | 46.7 ± 4.1 | 35.3 ± 3.0 | 144.1 ± 5.3 | 91.6 ± 8.5 |
| Red-leaf maple leaves | 97.4 ± 3.6 | 87.6 ± 3.7 | 166.0 ± 8.7 | 134.0 ± 12.1 |
| Sugar maple stems | 75.6 ± 5.1 | 55.7 ± 4.0 | 163.3 ± 8.1 | 111.1 ± 2.0 |
| Red-leaf maple stems | 159.3 ± 11.8 | 101.6 ± 8.9 | 183.8 ± 7.2 | 146.3 ± 9.0 |
| Sugar maple barks | 89.0 ± 4.9 | 52.2 ± 3.9 | 125.3 ± 6.0 | 91.7 ± 8.2 |
| Red-leaf maple barks | 82.4 ± 1.7 | 59.8 ± 1.9 | 104.6 ± 2.0 | 92.5 ± 2.3 |
| Sugar maple | 226.3 ± 7.9 | 165.3 ± 4.3 | 249.0 ± 6.5 | 178.3 ± 9.4 |
| sapwoods | | | | |
| Red-leaf maple | 173.5 ± 3.9 | 147.9 ± 3.0 | 188.9 ± 2.8 | 154.9 ± 2.5 |
| sapwoods | | | | |
| Sugar maple fruit | 223.4 ± 4.4 | 127.6 ± 3.2 | 258.4 ± 2.2 | 128.6 ± 2.8 |
| Red-leaf maple fruit | 244.8 ± 6.5 | 166.2 ± 7.9 | 269.1 ± 5.2 | 176.4 ± 6.2 |
| | Caco-2 | | CCD-18Co | |
| | 48 h | 72 h | 48 h | 72 h |
| Source | IC₅₀ ^{a} | IC₅₀ ^{a} | IC₅₀ ^{a} | IC₅₀ ^{a} |
| Sugar maple leaves | 127.0 ± 9.6 | 80.7 ± 4.7 | 305.7 ± 7.3 | 190.3 ± 9.3 |
| Red-leaf maple leaves | 149.1 ± 9.8 | 98.0 ± 5.1 | n.d. | 251.9 ± 6.3 |
| Sugar maple stems | 149.3 ± 8.6 | 101.2 ± 7.1 | 334.8 ± 9.8 | 220.6 ± 8.8 |
| Red-leaf maple stems | 170.2 ± 5.8 | 145.5 ± 7.4 | n.d. | 347.9 ± |
| | | | | 10.5 |
| Sugar maple barks | 100.6 ± 5.8 | 77.5 ± 3.8 | 235.8 ± 7.0 | 188.0 ± 5.2 |
| Red-leaf maple barks | 101.4 ± 2.6 | 85.8 ± 2.6 | n.d. | 191.1 ± 5.0 |
| Sugar maple | 243.0 ± 7.1 | 179.6 ± 4.9 | n.d. | 287.6 ± 8.5 |
| sapwoods | | | | |
| Red-leaf maple | 169.7 ± 4.3 | 137.2 ± 3.4 | 357.8 ± 7.0 | 252.9 ± 5.7 |
| sapwoods | | | | |
| Sugar maple fruit | 225.7 ± 2.4 | 120.4 ± 2.0 | 346.2 ± 3.1 | 263.5 ± 3.2 |
| Red-leaf maple fruit | 263.4 ± 7.0 | 183.3 ± 5.9 | n.d. | 283.8 ± 7.0 |

^{a} IC₅₀ (in μg/ml) is defined as the concentration required to achieve 50% inhibition over control cells (DMSO 0.5%); IC₅₀ values are shown as mean ± S.D. from three independent experiments.
n.d. not determined or not detected.

TABLE 6
Antiproliferative data for ginnalins A, B and C against human colon
cell lines after 48 and 72 h treatment

| | HCT-116 | | HT-29 | |
|---|---|---|---|---|
| | 48 h | 72 h | 48 h | 72 h |
| Compounds | IC₅₀^{a} | IC₅₀^{a} | IC₅₀^{a} | IC₅₀^{a} |
| Ginnalin A | 21.5 ± 1.6 | 16.3 ± 2.1 | 31.0 ± 2.6 | 24.1 ± 1.3 |
| Ginnalin B | 25.1 ± 1.8 | 20.0 ± 2.0 | 36.2 ± 1.5 | 27.3 ± 0.6 |
| Ginnalin C | 27.0 ± 1.9 | 22.3 ± 2.4 | 33.8 ± 2.0 | 30.1 ± 1.3 |
| | Caco-2 | | CCD-18Co | |
| | 48 h | 72 h | 48 h | 72 h |
| Compounds | IC₅₀^{a} | IC₅₀^{a} | IC₅₀^{a} | IC₅₀^{a} |
| Ginnalin A | 28.8 ± 1.8 | 21.7 ± 1.0 | n.d. | 46.6 ± 3.6 |
| Ginnalin B | 31.1 ± 1.9 | 22.6 ± 1.9 | n.d. | 47.1 ± 5.3 |
| Ginnalin C | 35.0 ± 1.4 | 29.8 ± 1.2 | n.d. | n.d. |

^{a}IC₅₀ (in μg/ml) is defined as the concentration required to achieve 50% inhibition over control cells (DMSO 0.5%); IC₅₀ values are shown as mean ± S.D. from three independent experiments.
n.d. not determined or not detected.

### Example 4

### Maplexins, New α-Glucosidase Inhibitors from Red Maple (Acer rubrum L.) Stems

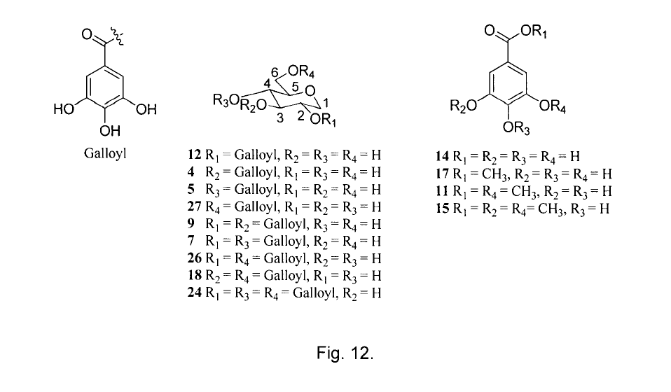

The Red maple extracts show promising α-glucosidase inhibitory activities with IC₅₀ values ranging from 4-10 μg/mL. This example shows the isolation and structural elucidation of five new gallotannins (compounds RMS 4, RMS 5, RMS 9, RMS 7, RMS 24), assigned the common name maplexins A-E, along with eight other known gallic acid derivatives (FIG. 12) from Red maple stems/twigs (RMS). Also, the antioxidant and α-glucosidase inhibitory properties, and the structure-activity relationship (SAR) of these compounds is described.

The dried stems of Red maple are extracted with methanol and fractionated with hexane, EtOAc and n-butanol. From the EtOAc extracts five new gallotannin compounds, along with eight known gallic acid derivatives are isolated by using a combination of chromatographic column separations.

Briefly, the dried stems of Red maple (500 g, dry) are ground and extracted exhaustively with methanol. The combined dried methanol extract is re-suspended in water and partitioned successively with n-hexane, EtOAc and n-butanol. The EtOAc fraction (18 g) is subjected to a silica gel chromatography column (CHCl₃/MeOH) to yield three fractions (A₁-A₃). Fraction A₃ (8 g) is chromatographed on a Sephadex LH-20 column and eluted with MeOH to give seven sub-fractions (B₁-B₇). Fraction B₄ is chromatographed on a C18MPLC column eluting with a gradient system of MeOH/H₂O (9:1 to 3:7, v/v) to afford 14 sub-fractions (C₁-C₁₄). Fraction C₂ is separated by semi-preparative HPLC eluted with MeOH/H₂O (20/80 v/v 3.2 mL/min) to yield compounds RMS2 (2.8 mg), RMS3 (2.5 mg) and gallic acid (460 mg). Fraction C₃ is separated by semi-preparative HPLC eluted with MeOH/H₂O (25/75 v/v 3.2 mL/min) to yield ginnalins B (18 mg) and C (9.2 mg). Fraction C₅ is separated by semi-preparative HPLC eluted with MeOH/H₂O (30/70 v/v 3.2 mL/min) to yield compound RMS5 (5.3 mg) and methyl gallate (7.7 mg). Fraction C₆ is separated by semi-preparative HPLC eluted with MeOH/H₂O (27/73 v/v 3.2 mL/min) to yield compound RMS6 (25 mg). Fraction C₉ is separated by semi-preparative HPLC eluted with MeOH/H₂O (25/75 v/v 3.2 mL/min) to yield ginnalin A (13 mg) and 3,4-dihydroxy-5-methoxybenzoic acid methyl ester (4.6 mg). Fraction C₁₂ is separated by semi-preparative HPLC eluted with MeOH/H₂O (41/59 v/v 3.2 mL/min) to yield methyl syringate (0.8 mg). Fraction B₆ is chromatographed on a C18MPLC column eluting with a gradient system of MeOH/H₂O (8:2 to 3:7, v/v) to afford 10 sub-fractions (D₁-D₁₀). Fraction D₁ is separated by semi-preparative HPLC eluted with MeOH/H₂O (30/70 v/v 3.2 mL/min) to yield 3,6-di-O-galloyl-1,5-anhydro-D-glucitol (1.4 mg). Fraction D₈ is separated by semi-preparative HPLC eluted with MeOH/H₂O (35/65 v/v 3.2 mL/min) to yield compound RMS9 (5 mg). Their structures are characterized using physicochemical and spectroscopic methods.

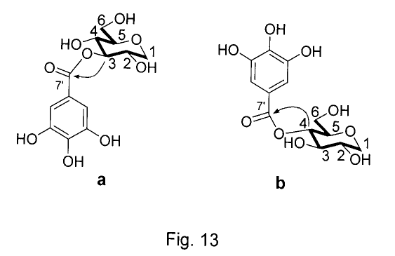

Compound RMS4, 3-O-galloyl-1,5-anhydro-D-glucitol: colorless amorphous solid; [α]²⁰_{D} +25 (c 0.280, MeOH); UV (MeOH) λₘₐₓ (log ε): 276 (4.10), 216 (4.41) nm; IR νₘₐₓ 1693, 1610 cm⁻¹; for ¹H NMR and ¹³C NMR data, see Table 7 and Table 8; HREIMS at m/z 315.0717 [M-H]⁻ (calcd for C₁₃H₁₅O₉, 315.0716) is a colorless amorphous solid, has a molecular formula of C₁₃H₁₆O₉ determined by HRESIMS at m/z 315.0717 [M-H]⁻ (calcd for C₁₃H₁₅O₉, 315.0716). Its IR absorptions implies the presence of ester carbonyl (1693) and aromatic ring (1610). The analysis of ¹H-NMR (Table 7) and ¹³C-NMR (Table 8) spectra showed typical galloyl signals at δ_{H} 7.14 (s, 2H), δ_{C} 167.1, 145.0 (2×C), 140.5, 120.5, 108.9 (2×C). Eight proton signals at δ_{H} 3.27-5.04 indicates the presence of a substructure similar to that of a sugar moiety. Apart from the galloyl carbon signals, six oxygenated carbon signals at δ_{C} 81.1, 79.8, 69.5, 68.6, 68.5 and 61.3 are observed in the ¹³C-NMR spectrum, which also supportes the presence of a sugar substructure. Further combined analysis of ¹H-¹H COSY, HSQC and HMBC spectrum allows the establishment of the structure of RMS4. The HSQC spectrum allows the assignment of all the protons to their bonding carbons. From the ¹H-¹H COSY spectrum, a 1-deoxysugar moiety (C-1 to C-6), drawn with bold bond in FIG. 13a, is established, and their relative stereochemistry is determined by the proton coupling constants (J_{1ax,2}=9.9 Hz, J_{2,3}=9.3 Hz, J_{3,4=9.4} Hz, J_{4,5}=9.5 Hz). Thus, a 1-deoxysugar moiety is determined as 1,5-anhydro-glucitol. The HMBC correlations between H-3 and ester carbonyl (C-7′) indicates that the galloyl group is linked at C-3 of 1,5-anhydro-glucitol. Acid hydrolysis of RMS4 afforded 1,5-anhydro-D-glucitol, which is identified by direct co-TLC comparison with an authentic sample. Therefore, compound RMS4 is elucidated as 3-O-galloyl-1,5-anhydro-D-glucitol assigned the common name maplexin A.

Compound RMS5, 4-O-galloyl-1,5-anhydro-D-glucitol: colorless amorphous solid; [α]²⁰_{D} +15 (c 0.060, MeOH); UV (MeOH) λₘₐₓ (log ε): 276 (4.10), 216 (4.41) nm; IR νₘₐₓ 1690, 1608 cm⁻¹; for ¹H NMR and ¹³C NMR data, see Table 7 and Table 8; HREIMS at m/z 315.0719 [M-H]⁻ (calcd for C₁₃H₁₅O₉, 315.0716) shows the same molecular formula as compound RMS2 (i.e. C₁₃H₁₆O₉ as per HRESIMS data) as well as similar UV and IR data. The ¹H- and ¹³C-NMR spectra indicates the presence of similar galloyl and 1,5-anhydro-glucitol substructures as RMS4. Further analysis of the ¹H-¹H COSY, HSQC and HMBC data found that the only difference between RMS4 and RMS5 is the linkage position connecting the galloyl and the 1,5-anhydro-glucitol moiety. The galloyl is eventually deduced to be attached at C-4 of the glucitol by the HMBC correlations from H-4 to C-7′. The D-configuration of the glucitol is determined by the similar acid hydrolysis method as for RMS4. Compound RMS5 is thus determined as 4-O-galloyl-1,5-anhydro-D-glucitol assigned the common name maplexin B.

Compound RMS9, 2,3-di-O-galloyl-1,5-anhydro-D-glucitol: colorless amorphous solid; [α]²⁰_{D} +13 (c 0.120, MeOH); UV (MeOH) λₘₐₓ (log ε): 276 (4.10), 216 (4.41) nm; IR νₘₐₓ 1705, 1600 cm⁻¹; for ¹H NMR and ¹³C NMR data, see Table 7 and Table 8; HREIMS at m/z 467.0826 [M-HT (calcd for C₂₀H₁₉O₁₃, 467.0826) is obtained as a colorless amorphous solid, shows the molecular formula of C₂₀H₂₀O₁₃ as determined by HRESIMS at m/z 467.0826 μM-H]⁻ (calcd for C₂₀H₁₉O₁₃, 467.0826). The ¹H- and ¹³C-NMR data shows were similar to those of compounds RMS4 and RMS5, indicating that the structures of both compounds are closely related, and the only difference is likely the presence of an additional galloyl moiety in RMS9. Further analysis of the 2D NMR data allows the establishment of the structure of RMS9. In the HMBC spectrum, the correlations from H-2 to C-7′, from H-3 to C-7″ indicated that the two galloyl groups are linked at C-2 and C-3 of 1,5-anhydro-glucitol, respectively. The D-configuration of the glucitol is determined by the same method as for compound RMS4. Compound RMS9 is therefore elucidated as 2,3-di-O-galloyl-1,5-anhydro-D-glucitol assigned the common name maplexin C.

Compound RMS7, 2,4-di-O-galloyl-1,5-anhydro-D-glucitol: colorless amorphous solid; [α]²⁰_{D} +6 (c 0.170, MeOH); UV (MeOH) λₘₐₓ (log ε): 276 (4.10), 216 (4.41) nm; IR νₘₐₓ 1703, 1601 cm⁻¹; for ¹H NMR and ¹³C NMR data, see Table 7 and Table 8; HREIMS at m/z 467.0821 [M-H]⁻ (calcd for C₂₀H₁₉O₁₃, 467.0826) has the same molecular formula (I.e. C₂₀H₂₀O₁₃) as compound RMS5 based on the HRESIMS at m/z 467.0821 [M-H]⁻ (calcd for C₂₀H₁₉O₁₃, 467.0826). The IR and UV spectrum are also similar to RMS5. Initial analyses the ¹H- and ¹³C-NMR data revealed the presence of two galloyl groups and a 1,5-anhydro-glucitol moiety. The difference between RMS7 and RMS9 is the linkage position of the galloyl with 1,5-anhydro-glucitol. The two galloyl groups are finally assigned to attachment at C-2 and C-4 of the 1,5-anhydro-glucitol on the basis of the HMBC correlations from H-2 to C-7′ and from H-4 to C-7″, respectively. The D-configuration of the glucitol is determined similar to that of compound RMS4. Compound RMS7 is thus elucidated as 2,4-di-O-galloyl-1,5-anhydro-D-glucitol assigned the common name maplexin D.

Compound 24, 2,4,6-tri-O-galloyl-1,5-anhydro-D-glucitol colorless amorphous solid; [α]²⁰_{D} +10 (c 0.130, MeOH); UV (MeOH) λₘₐₓ (log ε): 276 (4.10), 216 (4.41) nm; IR νₘₐₓ 1710, 1598 cm⁻¹; for ¹H NMR and ¹³C NMR data, see Table 1 and Table 2; HREIMS at m/z 619.0916 [M-H]⁻ (calcd for C₂₇H₂₃O₁₇, 619.0935) is obtained as colorless amorphous solid, shows the molecular formula of C₂₇H₂₄O₁₇ as determined by HRESIMS at m/z 619.0916 [M-H]⁻ (calcd for C₂₇H₂₃O₁₇, 619.0935). In the ¹H and ¹³C-NMR spectrum (Table 7 and Table 8, respectively), three sets of signals for galloyl moieties, eight proton signals at δ_{H} 3.46-5.22 and six oxygenated carbon signals at 6_{c} 76.8, 73.5, 71.8, 71.1, 66.6 and 62.7 are observed. The aforementioned spectral data suggests that compound RMS24 is similar to the above compounds, the only difference being the presence of three galloyl groups attached to the 1,5-anhydro-glucitol moiety. The HMBC correlations from H-2 to C-7′, from H-4 to C-7″, and from H₂-6 to C-7′″ indicates that the three galloyl groups are linked at C-2, C-4 and C-6 of the 1,5-anhydro-glucitol, respectively. The D-configuration of the glucitol is determined similar to that of RMS4. Compound RMS24 is thus elucidated as 2,4,6-tri-O-galloyl-1,5-anhydro-D-glucitol assigned the common name maplexin E.

Maplexins A-E, i.e. compounds RMS4, RMS 5, RMS 9, RMS 7 and RMS 24 (each 2 mg) are added to a mixture of concentrated HCl (0.5 mL), H₂O (2 mL) and dioxane (3 mL) and refluxed for 2 h, respectively. After completion of the reaction (monitored by TLC), the mixture is evaporated to dryness. The dry reaction mixture is partitioned between CHCl₃ and H₂O (3×5 mL). The aqueous layer is neutralized with Na₂CO₃ and then concentrated to dryness. The concentrate is dissolved in methanol and purified by Sephadex LH-20 chromatography to give 1,5-anhydro-D-glucitol, which is identified by co-TLC and specific rotation with the standard (Rf=0.43, CHCl₃-MeOH 10:1, positive value for optical rotation). The ESI-MS and ¹³C NMR spectrum (See supporting information) further supported the results.

Apart from the maplexins described herein, eight known compounds are identified as ginnalins B (RMS12), (Song, C. et al. 1982), C (RMS27), (Song, C. et al. 1982) and A (RMS26) (Bock, K, et al, 1980) 3,6-di-O-galloyl-1,5-anhydro-D-glucitol (RMS18), (Kumamoto, 1960) gallic acid (RMS14)(Cai, B et al, 2009), methyl gallate (RMS17), (Cai, B et al, 2009) 3,4-dihydroxy-5-methoxybenzoic acid methyl ester (RMS28)(Zhang, X et al 1991) and methyl syringate (RMS15) (Tan, J et al, 2005) on the basis of spectroscopic data (¹H, ¹³C NMR and ESIMS).Table 7. ¹H-NMR [δ, (Multiplicity, J_{HH} in Hertz)] Spectroscopic Data for Compounds 4, 5, 9, 7 and 24^{a}.

TABLE 7
¹H-NMR [δ, (Multiplicity, J_{HH} in Hertz)] Spectroscopic Data for
Compounds RMS4, RMS5, RMS9, RMS7 and RMS24^{a}.

| No. | RMS4 | RMS5 | RMS9 | RMS7 | RMS24 |
|---|---|---|---|---|---|
| 1ₐₓ | 3.27 (1H, dd, 10.3, | 3.24 (1H, dd, 10.8, | 3.45 (1H, dd, 10.9, | 3.40 (1H, dd, 10.9, | 3.46 (1H, dd, 11.5, |
| | 9.9) | 10.0) | 10.6) | 10.5) | 10.5) |
| 1_{eq} | 3.98 (1H, dd, 10.3, | 3.99 (1H, dd, 10.8, 5.0) | 4.20 (1H, dd, 10.9, 5.3) | 4.18 (1H, dd, 10.9, 5.7) | 4.22 (1H, dd, 11.5, 5.0) |
| | 5.8) | | | | |
| 2 | 3.71 (1H, ddd, 9.9, | 3.58 (1H, m) | 5.08 (1H, m) | 4.99 (1H, m) | 5.02 (1H, m) |
| | 9.3, 5.8) | | | | |
| 3 | 5.04 (1H, dd, 9.4, 9.3) | 3.45 (1H, m) | 5.40 (1H, dd, 9.5, 9.3) | 3.99 (1H, dd, 9.4, 9.3) | 4.03 (1H, dd, 9.3, 9.2) |
| 4 | 3.52 (1H, dd, 9.5, 9.4) | 4.89 (1H, overlap) | 3.69 (1H, dd, 10.0, 9.5) | 5.02 (1H, dd, 9.4, 8.9) | 5.22 (1H, dd, 9.8, 9.3) |
| 5 | 3.29 (1H, m) | 3.58 (1H, m) | 3.39 (1H, m) | 3.56 (1H, m) | 3.85 (1H, m) |
| 6 | 3.85 (1H, dd, 11.9, | 3.58 (1H, m) | 3.88 (1H, brd, 11.6) | 3.61 (1H, brd, 9.9) | 4.40 (1H, brd, 11.6) |
| | 1.8) | | | | |
| | 3.66 (1H, dd, 11.9, | 3.49 (1H, dd, 11.3, 6.2) | 3.72 (1H, dd, 11.6, 5.4) | 3.56 (1H, dd, 9.9, 5.6) | 4.19 (1H, dd, 11.6, 5.4) |
| | 5.4) | | | | |
| 2′, 6′ | 7.14 (2H, d, 1.5) | 7.09 (2H, d, 1.4) | 7.05 (2H, d, 1.2) | 7.09 (2H, d, 1.1) | 7.11 (2H, d, 2.1) |
| 2″, 6″ | | | 6.96 (2H, d, 1.4) | 7.11 (2H, d, 1.0) | 7.11 (2H, d, 2.1) |
| 2″′ | | | | | 7.08 (2H, d, 2.0) |
| 6″′ | | | | | |

^{a}Data were measured in CD₃OD at 500 MHz.

TABLE 8
¹³C-NMR (δ Values) Spectroscopic Data for Compounds RMS4,
RMS5, RMS9, RMS 7 and RMS 24^{a}

| No. | 4 | 5 | 9 | 7 | 24 |
|---|---|---|---|---|---|
| 1 | 69.5 | 69.6 | 66.4 | 66.6 | 66.6 |
| 2 | 68.5 | 70.2 | 70.0 | 71.9 | 71.8 |
| 3 | 79.8 | 79.5 | 76.5 | 73.5 | 73.5 |
| 4 | 68.6 | 71.4 | 68.5 | 71.4 | 71.1 |
| 5 | 81.1 | 76.4 | 81.3 | 79.6 | 76.8 |
| 6 | 61.3 | 61.4 | 61.2 | 61.2 | 62.7 |
| 1′ | 120.5 | 119.6 | 119.2 | 119.7 | 119.6 |
| 2′, 6′ | 108.9 | 108.9 | 108.9 | 108.9 | 108.9 |
| 3′, 5′ | 145.0 | 145.1 | 144.9 | 145.1 | 145.0 |
| 4′ | 140.5 | 138.4 | 138.4 | 138.6 | 138.6 |
| 7′ | 167.1 | 166.3 | 166.0 | 166.3 | 166.2 |
| 1″ | | | 120.0 | 119.6 | 119.6 |
| 2″, 6″ | | | 108.9 | 108.9 | 108.8 |
| 3″, 5″ | | | 145.0 | 145.1 | 145.0 |
| 4″ | | | 138.7 | 138.6 | 138.5 |
| 7″ | | | 166.8 | 166.2 | 166.0 |
| 1″′ | | | | | 119.8 |
| 2″′, 6″′ | | | | | 109.0 |
| 3″′, 5″′ | | | | | 145.1 |
| 4″′ | | | | | 138.7 |
| 7″′ | | | | | 166.7 |

^{a}Data were measured in CD₃OD at 125 MHz.

The α-glucosidase inhibitory properties and the structure-activity relationship (SAR) of all 13 compounds isolated from red maple stems is then investigated. Compounds RMS9, 7, 26, 18, 24 and RMS17 are found to be inhibitors of α-glucosidase enzyme in a concentration-dependent manner (Table 9). Compounds RMS12, 4, 5, and 27, which possess one galloyl group each, do not show any activity in this assay while compounds RMS9, 7, 26, and 18, which possess two galloyl groups each, shows moderate α-glucosidase inhibitory activity. Remarkably, Maplexin E, compound RMS24 which has three galloyl groups shows powerful α-glucosidase inhibitory activity in this assay. Maplexin E is 20 fold more potent than the known α-glucosidase inhibitory drug, acarbose (IC₅₀ 8.26 and 161.38 respectively).

TABLE 9
Antioxidant and alpha-glucosidase inhibitory activities of 13
compounds from red maple stems

| | IC₅₀ (μM) | |
|---|---|---|
| Compounds | DPPH | α-Glucosidase |
| 12 | 32.70 ± 0.48 | n.d. |
| 4 | 47.99 ± 1.11 | n.d. |
| 5 | 45.57 ± 1.45 | n.d. |
| 27 | 30.49 ± 0.80 | n.d. |
| 9 | 18.80 ± 0.77 | 1745.78 ± 168.05 |
| 7 | 18.59 ± 0.77 | 1221.84 ± 16.30 |
| 26 | 17.74 ± 0.21 | 95.38 ± 11.65 |
| 18 | 18.52 ± 0.44 | 88.42 ± 6.94 |
| 24 | 13.06 ± 0.16 | 8.26 ± 0.37 |
| 14 | 20.39 ± 0.34 | n.d. |
| 17 | 16.49 ± 0.26 | 317.39 ± 3.70 |
| 28 | 116.50 ± 4.98 | 6541.11 ± 19.90 |
| 15 | 990.57 ± 80.60 | n.d. |
| Vitamin C ^{b} | 71.02 ± 1.61 | — |
| BHT ^{b} | 1634.09 ± 16.07 | — |
| Acarbose ^{b} | — | 161.38 ± 5.5 |

^{a} IC₅₀ values are shown as mean ± S.D. from three independent experiments.
n.d. not detected.
^{b} Positive control.

The α-glucosidase inhibitory activities of compounds RMS9, 7, 26 and 18, with two galloyl groups each, also show significant differences in effects with IC₅₀ values of 1745.78, 1221.84, 95.38 and 88.42 μM, respectively. Compounds RMS26 and RMS18 showed stronger activities than compounds RMS9 and RMS7, which suggested that the α-glucosidase inhibitory activities of these gallotannins are influenced by both the number and positions of the galloyl groups. Thus, it is apparent that a galloyl group attached at the C-6 position of the glucitol moiety increased activity.

The antioxidant activities of 13 compounds are evaluated in the DPPH free radical scavenging assay. (Li and Seeram, 2011) All of isolates except RMS11 and RMS15 show better DPPH free radical scavenging activities than Vitamin C and BHT, the standard antioxidant materials (table 9). The IC₅₀ values of compounds RMS12, 4, 5, 27 are from 30.49 to 47.99 μM, compounds RMS9, 7, 26, and 18 are from 17.74 to 18.80 μM, and compound RMS24 is 13.06 μM. These results suggest that the antioxidant activity of gallotannins are influenced mainly by the number of the galloyl groups, while location of the galloyl group on the 1,5-anhydro-D-glucitol moiety had less influence on the antioxidant activity.

Thus, the identified new compounds from the Red maple species with α-glucosidase inhibitory potential include maplexin E (24), a natural agent that showed in vitro α-glucosidase inhibitory activity far superior to acarbose, a clinically available drug. Interestingly, our SAR study also indicates that these compounds may be synthetically manipulated with regards to the numbers and location of the galloyl groups on the 1,5-anhydro-α-glucitol moiety to enhance activity.

### Example 5

TABLE 10
List of compound from Red Maple Stems (RMS)

| NO. | name | Compound structure | MW |
|---|---|---|---|
| RMS 1 | catechin | 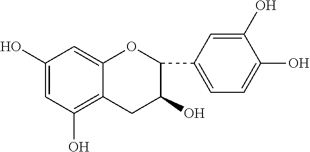 [SMILES: Oc1cc(O)c2c(c1)O[C@H](c1ccc(O)c(O)c1)[C@@H](O)C2] | 290 |
| RMS 2 | Epicatechin | 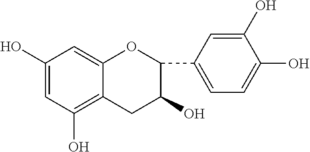 [SMILES: Oc1cc(O)c2c(c1)O[C@H](c1ccc(O)c(O)c1)[C@@H](O)C2] | 290 |
| RMS 3 | Nortrachelogenin 8′-O-β-D- glucopyranoside | 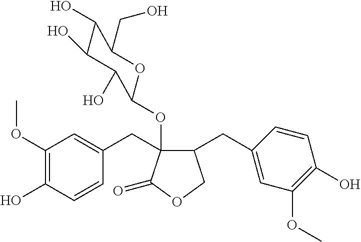 [SMILES: COc1cc(CC2COC(=O)C2(Cc2ccc(O)c(OC)c2)OC2OC(CO)C(O)C(O)C2O)ccc1O] | 536 |
| RMS 4 | 3-O-galloyl-1,5-anhydro-D- glucitol | 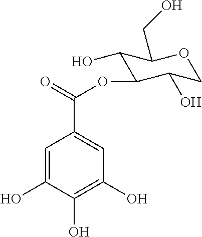 [SMILES: O=C(OC1C(O)COC(CO)C1O)c1cc(O)c(O)c(O)c1] | 316 |
| RMS 5 | 4-O-galloyl-1,5-anhydro-D- glucitol | 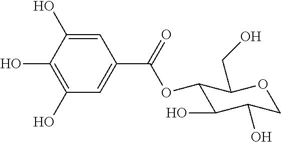 [SMILES: O=C(OC1C(CO)OCC(O)C1O)c1cc(O)c(O)c(O)c1] | 316 |
| RMS 6 | Galic acid | 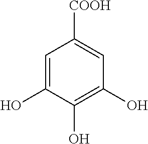 [SMILES: O=C(O)c1cc(O)c(O)c(O)c1] | 170 |
| RMS 7 | 2,4-di-O-galloyl-1,5-anhydro- D-glucitol | 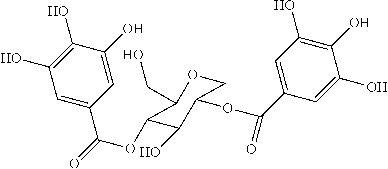 [SMILES: O=C(OC1COC(CO)C(OC(=O)c2cc(O)c(O)c(O)c2)C1O)c1cc(O)c(O)c(O)c1] | 468 |
| RMS 8 | Quercetin-3-O-α- rhamnopyranoside | 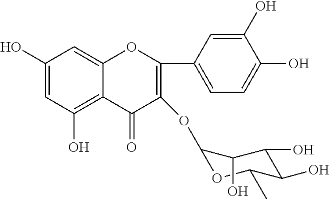 [SMILES: CC1OC(Oc2c(-c3ccc(O)c(O)c3)oc3cc(O)cc(O)c3c2=O)C(O)C(O)C1O] | 448 |
| RMS 9 | 2,3-di-O-galloyl-1,5-anhydro- D-glucitol | 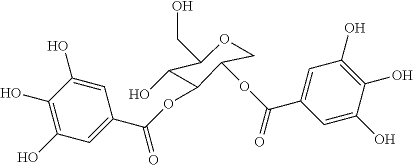 [SMILES: O=C(OC1COC(CO)C(O)C1OC(=O)c1cc(O)c(O)c(O)c1)c1cc(O)c(O)c(O)c1] | 468 |
| RMS 10 | 3-Methoxy-4-hydroxyphenol 1- O-β-D-(6′-O-galloyl)- glucopyranoside | 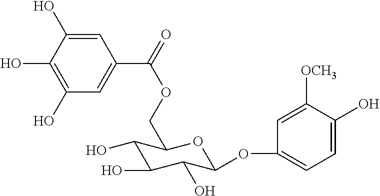 [SMILES: COc1cc(OC2OC(COC(=O)c3cc(O)c(O)c(O)c3)C(O)C(O)C2O)ccc1O] | 454 |
| RMS 11 | 3,5-Dihydroxy-4- methoxybenzoic acid methyl ester | 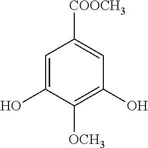 [SMILES: COC(=O)c1cc(O)c(OC)c(O)c1] | 198 |
| RMS 12 | Ginnalins A 2,6-di-O-galloyl-1,5-anhydro- D-glucitol | 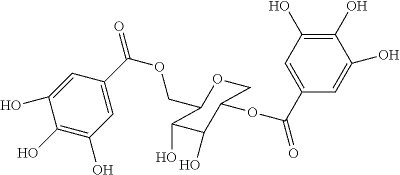 [SMILES: O=C(OCC1OCC(OC(=O)c2cc(O)c(O)c(O)c2)C(O)C1O)c1cc(O)c(O)c(O)c1] | 468 |
| RMS 13 | 7,8-Dihydroxy-6- methoxycoumarin | 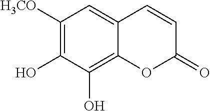 [SMILES: COc1cc2ccc(=O)oc2c(O)c1O] | 208 |
| RMS 14 | Gallic acid 4-methyl ester 4-Methoxy-3,5- dihydroxybenzoic acid | 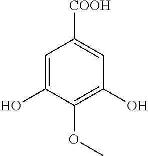 | 184 |
| RMS 15 | Methyl syringate Methyl 3,5-dimethoxy-4- hydroxybenzoate | 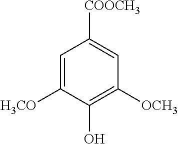 [SMILES: COC(=O)c1cc(OC)c(O)c(OC)c1] | 212 |
| RMS 16 | Methyl vanillate | 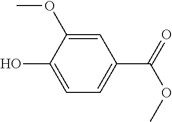 | 182 |
| RMS 17 | Methyl gallate | 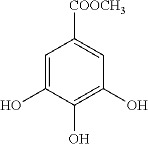 [SMILES: COC(=O)c1cc(O)c(O)c(O)c1] | 184 |
| RMS 18 | 3,6-di-O-galloyl-1,5-anhydro- D-glucitol | 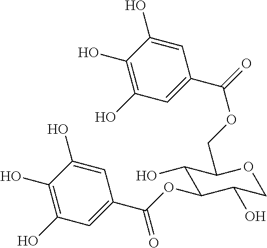 [SMILES: O=C(OCC1OCC(O)C(OC(=O)c2cc(O)c(O)c(O)c2)C1O)c1cc(O)c(O)c(O)c1] | 468 |
| RMS 19 | Procyanidin A₆ (+)-Epicatechin-(4β-8,2β-O-7)- catechin | 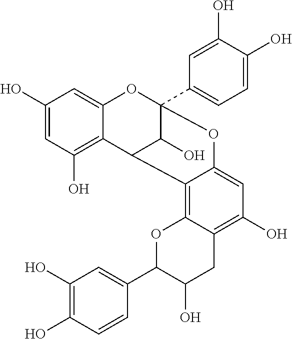 [SMILES: Oc1cc(O)c2c(c1)O[C@@]1(c3ccc(O)c(O)c3)Oc3cc(O)c4c(c3C2C1O)OC(c1ccc(O)c(O)c1)C(O)C4] | |
| RMS 20 | Epicatechin gallate | 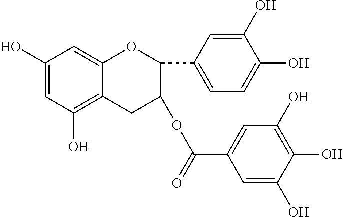 [SMILES: O=C(OC1Cc2c(O)cc(O)cc2O[C@@H]1c1ccc(O)c(O)c1)c1cc(O)c(O)c(O)c1] | 442 |
| RMS 21 | Procyanidin A₂; (+)-Epicatechin-(4β-8,2β-O-7)- epicatechin | 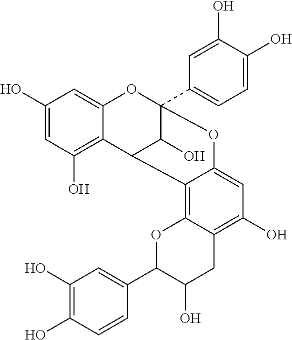 [SMILES: Oc1cc(O)c2c(c1)O[C@@]1(c3ccc(O)c(O)c3)Oc3cc(O)c4c(c3C2C1O)OC(c1ccc(O)c(O)c1)C(O)C4] | 576 |
| RMS 22 | 3″-Galloylquercitrin; Querctin-3-O-(3″-O-galloyl)- α-rhamnopyranoside | 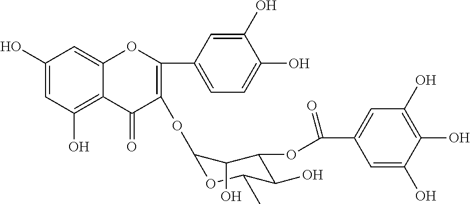 [SMILES: CC1OC(Oc2c(-c3ccc(O)c(O)c3)oc3cc(O)cc(O)c3c2=O)C(O)C(OC(=O)c2cc(O)c(O)c(O)c2)C1O] | 600 |
| RMS 23 | 2″-Galloylquercitrin; Quercetin-3-O-(2″-O-galloyl)- α-rhamnopyranoside | 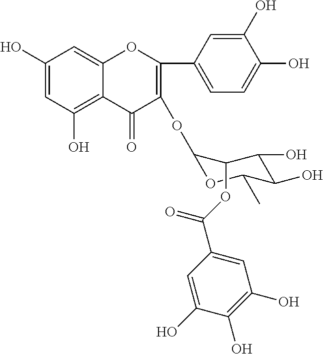 [SMILES: CC1OC(Oc2c(-c3ccc(O)c(O)c3)oc3cc(O)cc(O)c3c2=O)C(OC(=O)c2cc(O)c(O)c(O)c2)C(O)C1O] | 600 |
| RMS 24 | 2,4,6-tri-O-galloyl-1,5-anhydro- D-glucitol | 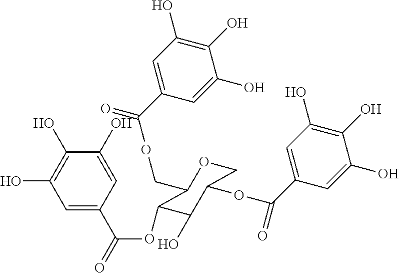 [SMILES: O=C(OCC1OCC(OC(=O)c2cc(O)c(O)c(O)c2)C(O)C1OC(=O)c1cc(O)c(O)c(O)c1)c1cc(O)c(O)c(O)c1] | 620 |
| RMS 26 | Ginnalins B 6-O-galloyl-1,5-anhydro-D- glucitol | 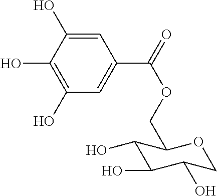 [SMILES: O=C(OCC1OCC(O)C(O)C1O)c1cc(O)c(O)c(O)c1] | 316 |
| RMS 27 | Ginnalins C 2-O-galloyl-1,5-anhydro-D- glucitol | 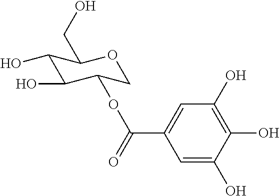 [SMILES: O=C(OC1COC(CO)C(O)C1O)c1cc(O)c(O)c(O)c1] | 316 |
| RMS 28 | 3,4-dihydroxy-5- methoxybenzoic acid methyl ester | 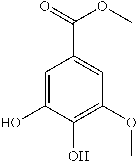 | |

### Example 6

TABLE 11
List of compounds from Red Maple Bark (RMB)

| No. | Structure | M.W. |
|---|---|---|
| RMB 1 | 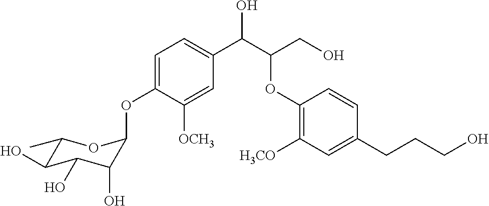 [SMILES: COc1cc(CCCO)ccc1OC(CO)C(O)c1ccc(OC2OC(C)C(O)C(O)C2O)c(OC)c1] erythro | C26H36O11 524.2258 |
| RMB 2 | 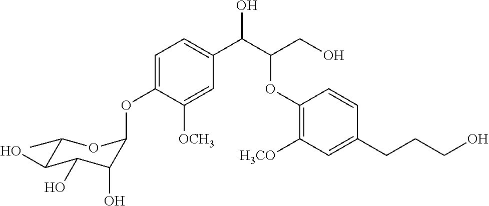 [SMILES: COc1cc(CCCO)ccc1OC(CO)C(O)c1ccc(OC2OC(C)C(O)C(O)C2O)c(OC)c1] threo | C26H36O11 524.2258 |
| RMB 3 | 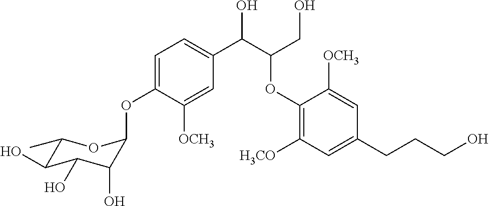 [SMILES: COc1cc(C(O)C(CO)Oc2c(OC)cc(CCCO)cc2OC)ccc1OC1OC(C)C(O)C(O)C1O] | C27H38O12 554.2363 |
| RMB 4 | 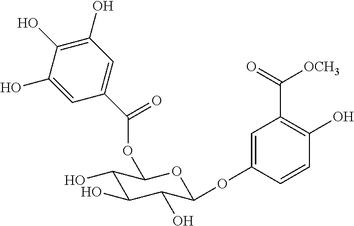 [SMILES: COC(=O)c1cc(OC2OC(OC(=O)c3cc(O)c(O)c(O)c3)C(O)C(O)C2O)ccc1O] | C21H22O13 482.1060 |
| RMB 5 | 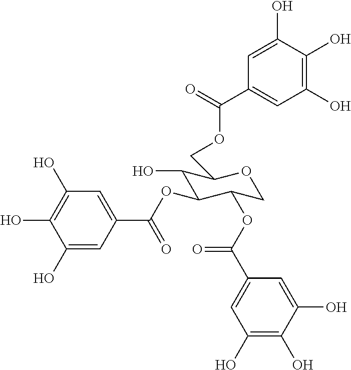 [SMILES: O=C(OCC1OCC(OC(=O)c2cc(O)c(O)c(O)c2)C(OC(=O)c2cc(O)c(O)c(O)c2)C1O)c1cc(O)c(O)c(O)c1] | C27H24O17 620.1013 |
| RMB 6 | 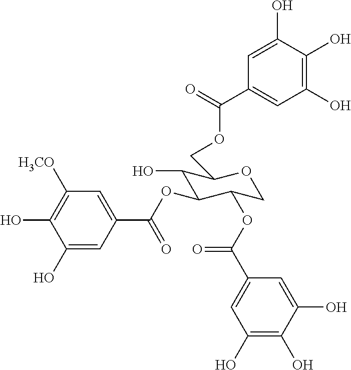 [SMILES: COc1cc(C(=O)OC2C(OC(=O)c3cc(O)c(O)c(O)c3)COC(COC(=O)c3cc(O)c(O)c(O)c3)C2O)cc(O)c1O] | C28H26O17 634.1170 |
| RMB 7 | 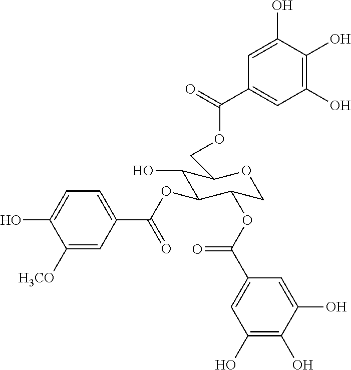 [SMILES: COc1cc(C(=O)OC2C(OC(=O)c3cc(O)c(O)c(O)c3)COC(COC(=O)c3cc(O)c(O)c(O)c3)C2O)ccc1O] | C28H26O16 618.1221 |
| RMB 8 | 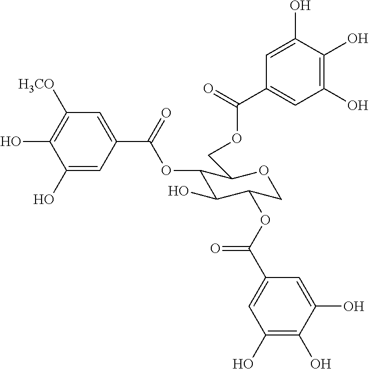 [SMILES: COc1cc(C(=O)OC2C(COC(=O)c3cc(O)c(O)c(O)c3)OCC(OC(=O)c3cc(O)c(O)c(O)c3)C2O)cc(O)c1O] | C28H26O17 634.1170 |

### Example 7

TABLE 12
List of compounds from Sugar Maple bark (SMB)

| No | Compound structure and name | Molecular formula |
|---|---|---|
| SMB 1 | 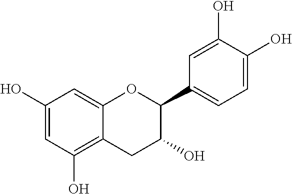 [SMILES: Oc1cc(O)c2c(c1)O[C@@H](c1ccc(O)c(O)c1)[C@H](O)C2] Catechin | C₁₅H₁₄O₆ Mw = 290 |
| SMB 2 | 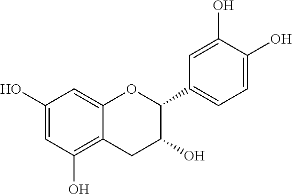 [SMILES: Oc1cc(O)c2c(c1)O[C@H](c1ccc(O)c(O)c1)[C@H](O)C2] Epicatechin | C₁₅H₁₄O₆ Mw = 290 |
| SMB 3 | 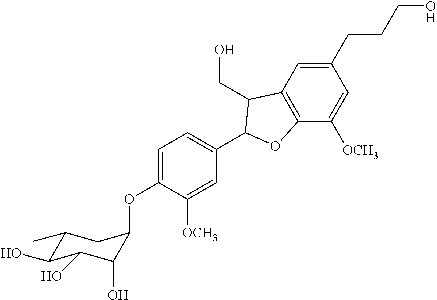 [SMILES: COc1cc(C2Oc3c(OC)cc(CCCO)cc3C2CO)ccc1OC1CC(C)C(O)C(O)C1O] Icariside E₄ | C₂₇H₃₆O₉ Mw = 504 |
| SMB 4 | 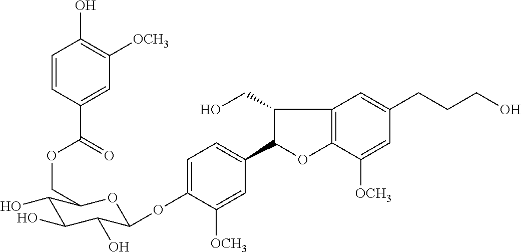 New | C₃₄H₄₀O₁₄ Mw = 672 |
| SMB 5 | 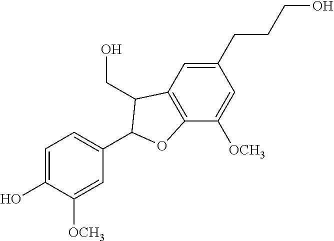 [SMILES: COc1cc(C2Oc3c(OC)cc(CCCO)cc3C2CO)ccc1O] Dihydrodehydrodiconiferyl alcohol | C₂₀H₂₄O₆ Mw = 360 |
| SMB 6 | 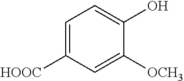 [SMILES: COc1cc(C(=O)O)ccc1O] Vanillic acid | C₈H₈O₄ Mw = 168 |
| SMB 7 | 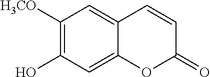 [SMILES: COc1cc2ccc(=O)oc2cc1O] Scopoletin | C₁₀H₈O₄ Mw = 192 |
| SMB 8 | 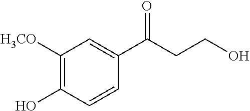 [SMILES: COc1cc(C(=O)CCO)ccc1O] 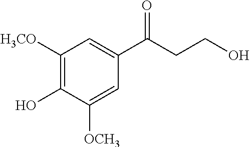 [SMILES: COc1cc(C(=O)CCO)cc(OC)c1O] | C₁₀H₁₂O₄ Mw = 176 C₁₁H₁₄O₅ Mw = 226 |
| SMB 9 | 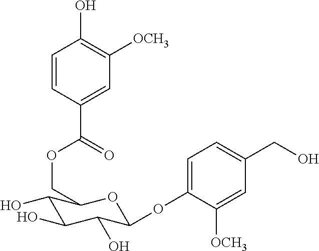 [SMILES: COc1cc(C(=O)OCC2OC(Oc3ccc(CO)cc3OC)C(O)C(O)C2O)ccc1O] New | C₂₂H₂₆O₁₁ Mw = 466 |
| SMB 10 | 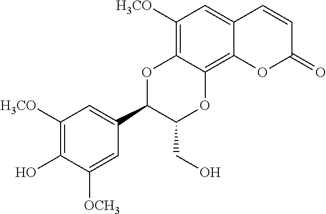 [SMILES: COc1cc([C@H]2Oc3c(OC)cc4ccc(=O)oc4c3O[C@@H]2CO)cc(OC)c1O] Cleomiscosin C | C₂₁H₂₀O₉ Mw = 416 |
| SMB 11 | 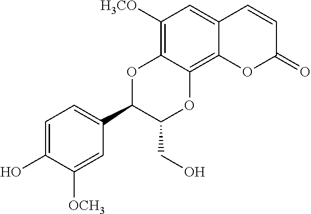 [SMILES: COc1cc([C@H]2Oc3c(OC)cc4ccc(=O)oc4c3O[C@@H]2CO)ccc1O] Cleomiscosin A | C₂₀H₁₈O₈ Mw = 386 |
| SMB 12 | 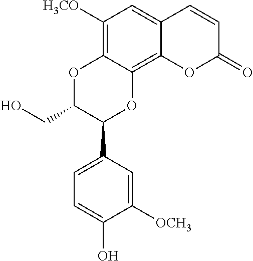 [SMILES: COc1cc([C@@H]2Oc3c(c(OC)cc4ccc(=O)oc34)O[C@H]2CO)ccc1O] Cleomiscosin B | C₂₀H₁₈O₈ Mw = 386 |
| SMB 13 | 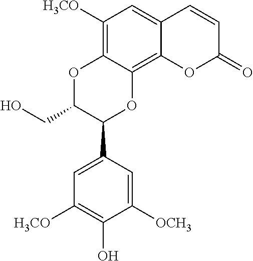 [SMILES: COc1cc([C@@H]2Oc3c(c(OC)cc4ccc(=O)oc34)O[C@H]2CO)cc(OC)c1O] Cleomiscosin D | C₂₁H₂₀O₉ Mw = 416 |
| SMB 14 | 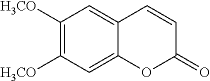 [SMILES: COc1cc2ccc(=O)oc2cc1OC] | C₁₁H₁₀O₄ Mw = 206 |
| SMB 15 | 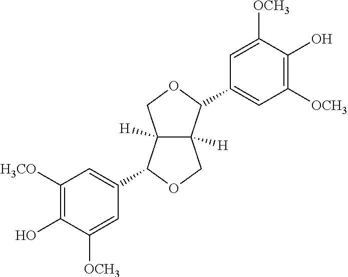 | C₂₂H₂₆O₈ Mw = 418 |
| SMB 16 | 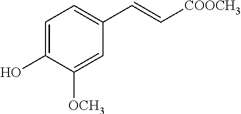 [SMILES: COC(=O)/C=C/c1ccc(O)c(OC)c1] | C₁₁H₁₂O₄ Mw = 208 |
| SMB 17 | 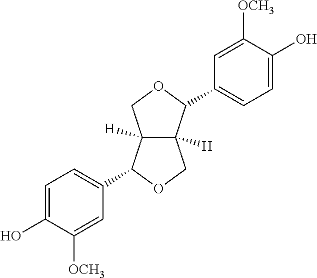 | C₂₀H₂₂O₆ Mw = 358 |
| SMB 18 | 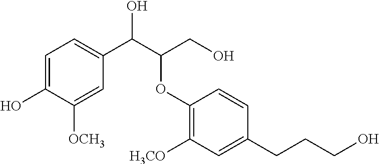 [SMILES: COc1cc(C(O)C(CO)Oc2ccc(CCCO)cc2OC)ccc1O] | C₂₀H₂₆O₇ Mw = 378 |
| SMB 19 | 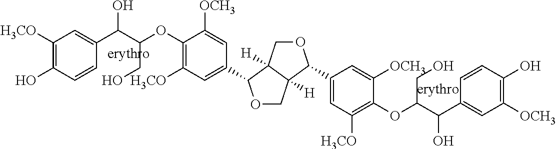 [SMILES: COc1cc(C(O)C(CO)Oc2c(OC)cc([C@H]3OC[C@H]4[C@@H]3CO[C@@H]4c3cc(OC)c(OC(CO)C(O)c4ccc(O)c(OC)c4)c(OC)c3)cc2OC)ccc1O] | C₄₂H₅₀O₁₆ Mw = 810 |
| SMB 20 | 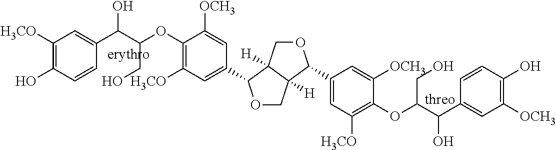 [SMILES: COc1cc(C(O)C(CO)Oc2c(OC)cc([C@H]3OC[C@H]4[C@@H]3CO[C@@H]4c3cc(OC)c(OC(CO)C(O)c4ccc(O)c(OC)c4)c(OC)c3)cc2OC)ccc1O] | C₄₂H₅₀O₁₆ Mw = 810 |
| SMB 21 | 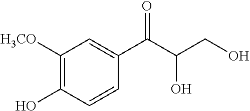 [SMILES: COc1cc(C(=O)C(O)CO)ccc1O] | C₁₀H₁₂O₅ Mw = 212 |
| SMB 22 | 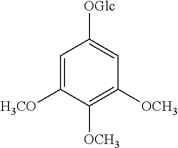 Koaburside | C₁₅H₂₂O₉ Mw = 346 |
| SMB 23 | 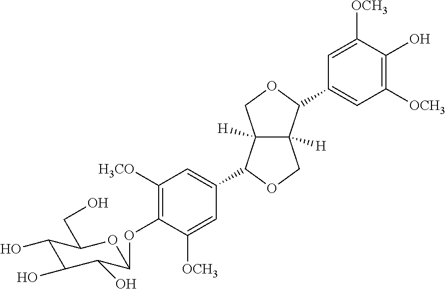 | |
| SMB 24 | 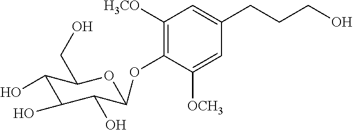 [SMILES: COc1cc(CCCO)cc(OC)c1OC1OC(CO)C(O)C(O)C1O] 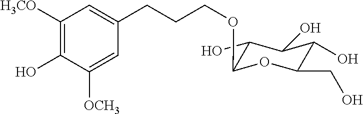 [SMILES: COc1cc(CCCOC2OC(CO)C(O)C(O)C2O)cc(OC)c1O] | |
| SMB 25 | 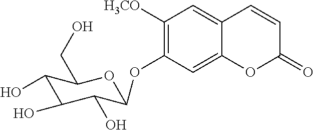 [SMILES: COc1cc2ccc(=O)oc2cc1OC1OC(CO)C(O)C(O)C1O] | |
| SMB 26 | 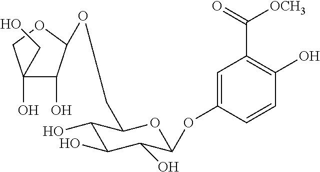 [SMILES: COC(=O)c1cc(OC2OC(COC3OCC(O)(CO)C3O)C(O)C(O)C2O)ccc1O] | |
| SMB 27 | 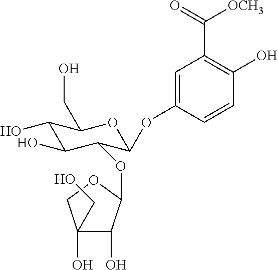 [SMILES: COC(=O)c1cc(OC2OC(CO)C(O)C(O)C2OC2OCC(O)(CO)C2O)ccc1O] | |
| SMB 28 | 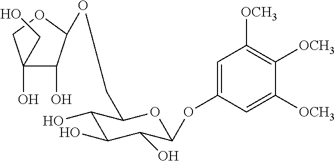 [SMILES: COc1cc(OC2OC(COC3OCC(O)(CO)C3O)C(O)C(O)C2O)cc(OC)c1OC] | |
| SMB 29 | 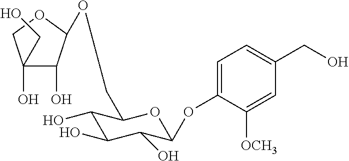 [SMILES: COc1cc(CO)ccc1OC1OC(COC2OCC(O)(CO)C2O)C(O)C(O)C1O] | |
| SMB 30 | 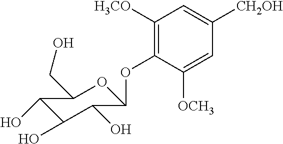 [SMILES: COc1cc(CO)cc(OC)c1OC1OC(CO)C(O)C(O)C1O] | |
| SMB 31 | 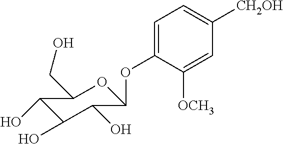 [SMILES: COc1cc(CO)ccc1OC1OC(CO)C(O)C(O)C1O] | |

While preferred embodiments have been described above and illustrated in the accompanying drawings, it will be evident to those skilled in the art that modifications may be made without departing from this disclosure. Such modifications are considered as possible variants comprised in the scope of the disclosure.

1. A composition for the prophylaxis of an ailment comprising a therapeutically effective amount of an extract of an Acer tree in association with a pharmaceutically acceptable carrier.

2. The composition according to claim 1, wherein said extract of Acer tree is an extract from a non-concentrated or concentrated sap, a samara fruit, a samara seed, a stems of leaf, a stem of a samara, a twig, a root, a leaf, a bark, a heartwood, a sapwood, a whole branch, a bark of a branch, a wood of a branch, a sugar, a syrup, a syrup extract, a syrup-derived product, a rejection of syrup or syrup-derived product production, a residue of syrup or syrup-derived product production, or combinations thereof.

3. The composition according to any one of claims 1-2, wherein said Acer tree is chosen from Acer nigrum, Acer lanurn, Acer acuminatum, Acer albopurpurascens, Acer argutum, Acer barbinerve, Acer buergerianum, Acer caesium, Acer campbeffii, Acer campestre, Acer capillipes, Acer cappadocicum, Acer carpinifolium, Acer caudatifolium, Acer caudatum, Acer cinnamomifolium, Acer circinatum, Acer cissifolium, Acer crassum, Acer crataegifolium, Acer davidii, Acer decandrum, Acer diabolicum, Acer distylum, Acer divergens, Acer erianthum, Acer erythranthum, Acer fabri, Acer garrettii, Acer glabrum, Acer grandidentatum, Acer griseum, Acer heldreichii, Acer henryi, Acer hyrcanum, Acer ibericum, Acer japonicum, Acer kungshanense, Acer kweilinense, Acer laevigatum, Acer laurinum, Acer lobelii, Acer lucidum, Acer macrophyllum, Acer mandshuricum, Acer maximowiczianum, Acer miaoshanicum, Acer micranthum, Acer miyabei, Acer mono, Acer mono×Acer truncatum, Acer monspessulanum, Acer negundo, Acer ningpoense, Acer nipponicum, Acer oblongum, Acer obtusifolium, Acer oliverianum, Acer opalus, Acer palmatum, Acer paxii, Acer pectinatum, Acer pensylvanicum, Acer pentaphyllum, Acer pentapomicum, Acer pictum, Acer pilosum, Acer platanoides, Acer poliophyllum, Acer pseudoplatanus, Acer pseudosieboldianum, Acer pubinerve, Acer pycnanthum, Acer rubrum, Acer rufinerve, Acer saccharinum, Acer saccharum, Acer sempervirens, Acer shirasawanum, Acer sieboldianum, Acer sinopurpurescens, Acer spicatum, Acer stachyophyllum, Acer sterculiaceum, Acer takesimense, Acer tataricum, Acer tegmentosum, Acer tenuifolium, Acer tetramerum, Acer trautvetteri, Acer triflorum, Acer truncatum, Acer tschonoskii, Acer turcomanicum, Acer ukurunduense, Acer velutinum, Acer wardii, Acer x peronai, and Acer x pseudoheldreichii.

4. The composition according to any one of claims 1-2, wherein said Acer tree is chosen from Acer Saccharum and Acer Rubrum L.

5. The composition according to claim 2, wherein the syrup—derived product comprises butter, granulated sugar, hardened sugar, soft sugar, taffy, flakes an extract from lyophilisation of a sap, a maple concentrate or a maple syrup, an extract from drying of a sap, a maple concentrate or a maple syrup, an extract from crystallization of a sap, a maple concentrate or a maple syrup, an extract from pulverization of a sap, a maple concentrate or a maple syrup, an extract from atomization of a sap, a maple concentrate or a maple syrup, an extract from centrifugation of a sap, a maple concentrate or a maple syrup, or combinations thereof.

6. The composition according to claim 2, wherein the syrup extract is chosen from a methanol extract, a butanol extract, a butanol extract with sugar, a butanol extract without sugar, an ethyl acetate extract, an ethanol extract, a 95% ethanol/5% hot water extract, or combinations thereof.

7. The composition according to claim 1, wherein the extract of Acer tree comprises an extract from concentrated Acer tree water issued from reverse osmosis, a concentrated Acer tree water issued from pre-boiling nanofiltration, a pasteurized Acer tree water, a sterilized Acer tree water, an Acer tree water issued from a high pressure processing, an Acer tree water sterilized by UV irradiation, an Acer tree water sterilized by microwave irradiation or combinations thereof.

8. The composition according to claim 1, wherein the extract of Acer tree comprises an Acer tree molecule.

9. The composition according to claim 8, wherein the Acer tree molecule comprises:
Lyoniresinol,
Isolariciresinol,
secoisolariciresinol,
Dehydroconiferyl alcohol,
5′-methoxy-dehydroconiferyl alcohol,
erythro-guaiacylglycerol-β-O-4′-coniferyl alcohol,
erythro-guaiacylglycerol-β-O-4′-dihydroconiferyl alcohol,
[3-[4-[(6-deoxy-α-L-mannopyranosyl)oxy]-3-methoxyphenyl]methyl]-5-(3,4-dimethoxyphenyl)dihydro-3-hydroxy-4-(hydroxymethyl)-2(3H)-furanone,
5-(3″,4″-dimethoxyphenyl)-3-hydroxy-3-(4′-hydroxy-3′-methoxybenzyl)-4-hydroxymethyl-dihydrofuran-2-one,
Scopoletin,
Fraxetin,
lsofraxidin,
Gallic acid,
Ginnalin A (acertannin),
Syringic acid,
Ginnalin B,
Ginnalin C,
Trimethyl gallic acid methyl ester
(E)-3,3′-dimethoxy-4,4′-dihydroxy stilbene,
p-coumaric acid,
Ferulic acid,
(E)-Coniferol,
Syringenin,
Dihydroconiferyl alcohol,
C-veratroylglycol,
2,3-dihydroxy-1-(3,4-dihydroxyphenyl)-1-propanone
2,3-Dihydroxy-1-(4-hydroxy-3,5-dimethoxyphenyl)-1-propanone,
3-Hydroxy-1-(4-hydroxy-3,5-dimethoxyphenyl)propan-1-one,
3′,4′,5′-Trihydroxyacetophenone,
4-Acetylcatechol,
2,4,5-Trihydroxyacetophenone,
1-(2,3,4-trihydroxy-5-methylphenyl)-ethanone,
2-Hydroxy-3′,4′-dihydroxyacetophenone,
Vanillin,
Syringaldehyde,
Catechaldehyde,
3,4-Dihydroxy-2-methylbenzaldehyde,
Catechol,
Catechin,
Epicatechin,
Quebecol,
(erythro, erythro)-1-[4-[2-hydroxy-2-(4-hydroxy-3-methoxyphenyl)-1-(hydroxymethyl)ethoxy]-3,5-dimethoxyphenyl]-1,2,3-propanetriol,
(erythro, threo)-1-[4-[2-hydroxy-2-(4-hydroxy-3-methoxyphenyl)-1-(hydroxymethyl)ethoxy]-3,5-dimethoxyphenyl]-1,2,3-propanetriol,
(threo, erythro)-1-[4-[(2-hydroxy-2-(4-hydroxy-3-methoxyphenyl)-1-(hydroxymethyl)ethoxy]-3-methoxyphenyl]-1,2,3-propanetriol,
(threo, threo)-1-[4-[(2-hydroxy-2-(4-hydroxy-3-methoxyphenyl)-1-(hydroxymethyl)ethoxy]-3-methoxyphenyl]-1,2,3-propanetriol,
threo-guaiacylglycerol-β-O-4′-dihydroconiferyl alcohol,
erythro-1-(4-hydroxy-3-methoxyphenyl)-2-[4-(3-hydroxypropyl)-2,6-dimethoxyphenoxy]-1,3-propanediol,
2-[4-[2,3-dihydro-3-(hydroxymethyl)-5-(3-hydroxypropyl)-7-methoxy-2-benzofuranyl]-2,6-dimethoxyphenoxy]-1-(4-hydroxy-3-methoxyphenyl)-1,3-propanediol,
Acerkinol,
Leptolepisol D,
Buddlenol E,
(1S,2R)-2-[2,6-dimethoxy-4-[(1S,3aR,4S,6aR)-tetrahydro-4-(4-hydroxy-3,5-dimethoxyphenyl)-1H,3H-furo[3,4-c]furan-1-yl]phenoxy]-1-(4-hydroxy-3-methoxyphenyl)-1,3-propanediol,
Syringaresinol,
Icariside E4,
Sakuraresinol,
1,2-diguaiacyl-1,3-propanediol
protocatechuic acid,
4-(dimethoxymethyl)-pyrocatechol,
Tyrosol,
4-hydroxycatechol,
Phaseic acid,
Nortrachelogenin 8′-O-β-D-glucopyranoside,
3-O-galloyl-1,5-anhydro-D-glucitol,
4-O-galloyl-1,5-anhydro-D-glucitol,
2,4-di-O-galloyl-1,5-anhydro-D-glucitol,
Quercetin-3-O-α-rhamnopyranoside,
2,3-di-O-galloyl-1,5-anhydro-D-glucitol,
3-Methoxy-4-hydroxyphenol 1-O-β-D-(6′-O-galloyl)-glucopyranoside,
3,5-Dihydroxy-4-methoxybenzoic acid methyl ester,
7,8-Dihydroxy-6-methoxycoumarin,
4-Methoxy-3,5-dihydroxybenzoic acid,
Methyl 3,5-dimethoxy-4-hydroxybenzoate,
Methyl vanillate,
Methyl gallate,
3,6-di-O-galloyl-1,5-anhydro-D-glucitol,
(+)-Epicatechin-(4β-8,2β-O-7)-catechin,
Epicatechin gallate,
(+)-Epicatechin-(4β-8,2β-O-7)-epicatechin,
Quercetin-3-O-(3″-O-galloyl)-α-rhamnopyranoside,
Quercetin-3-O-(2″-O-galloyl)-α-rhamnopyranoside,
2,4,6-tri-O-galloyl-1,5-anhydro-D-glucitol,

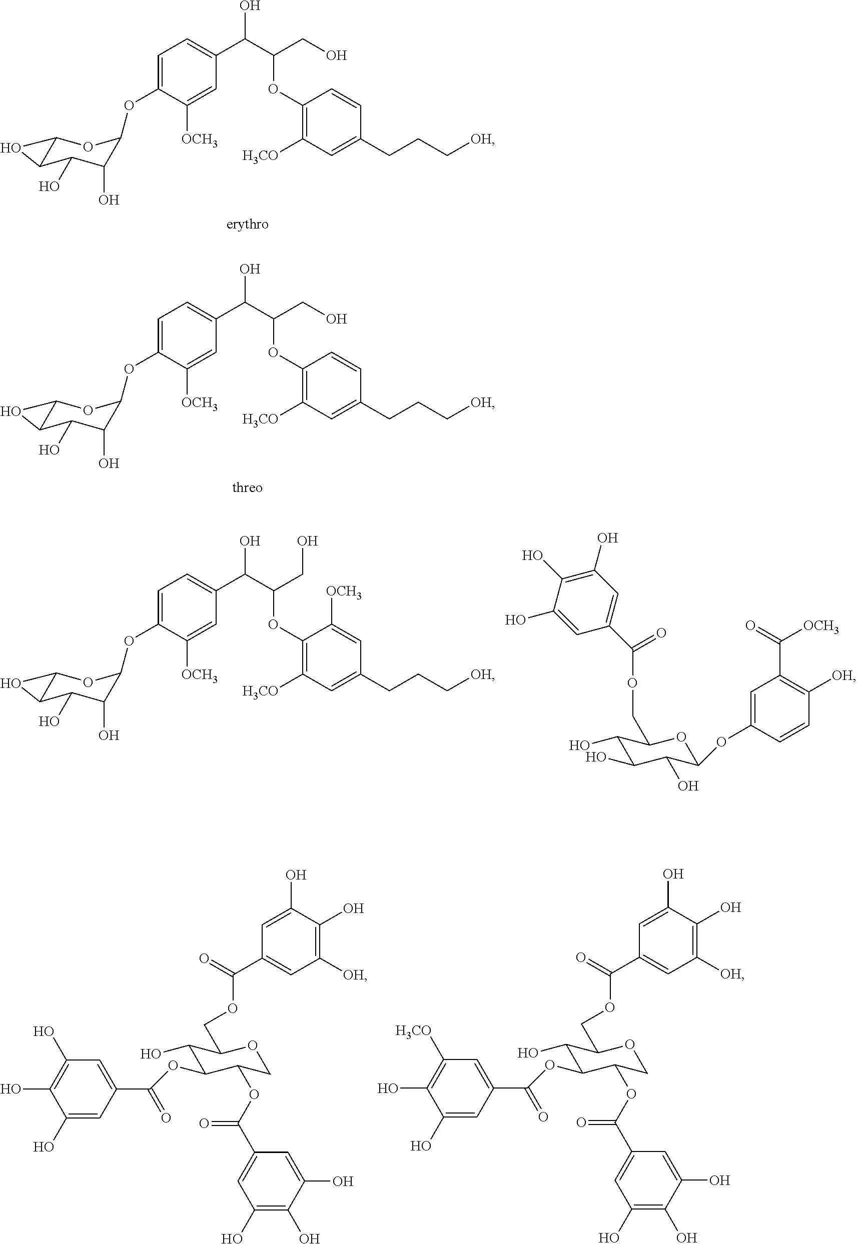 [SMILES: COc1cc(CCCO)ccc1OC(CO)C(O)c1ccc(OC2OC3OC3C(O)C2O)c(OC)c1 ; COc1cc(CCCO)ccc1OC(CO)C(O)c1ccc(OC2OC3OC3C(O)C2O)c(OC)c1 ; COc1cc(C(O)C(CO)Oc2c(OC)cc(CCCO)cc2OC)ccc1OC1OC2OC2C(O)C1O ; COC(=O)c1cc(OC2OC(COC(=O)c3cc(O)c(O)c(O)c3)C(O)C(O)C2O)ccc1O ; O=C(OCC1OCC(OC(=O)c2cc(O)c(O)c(O)c2)C(OC(=O)c2cc(O)c(O)c(O)c2)C1O)c1cc(O)c(O)c(O)c1 ; COc1cc(C(=O)OC2C(OC(=O)c3cc(O)c(O)c(O)c3)COC(COC(=O)c3cc(O)c(O)c(O)c3)C2O)cc(O)c1O]

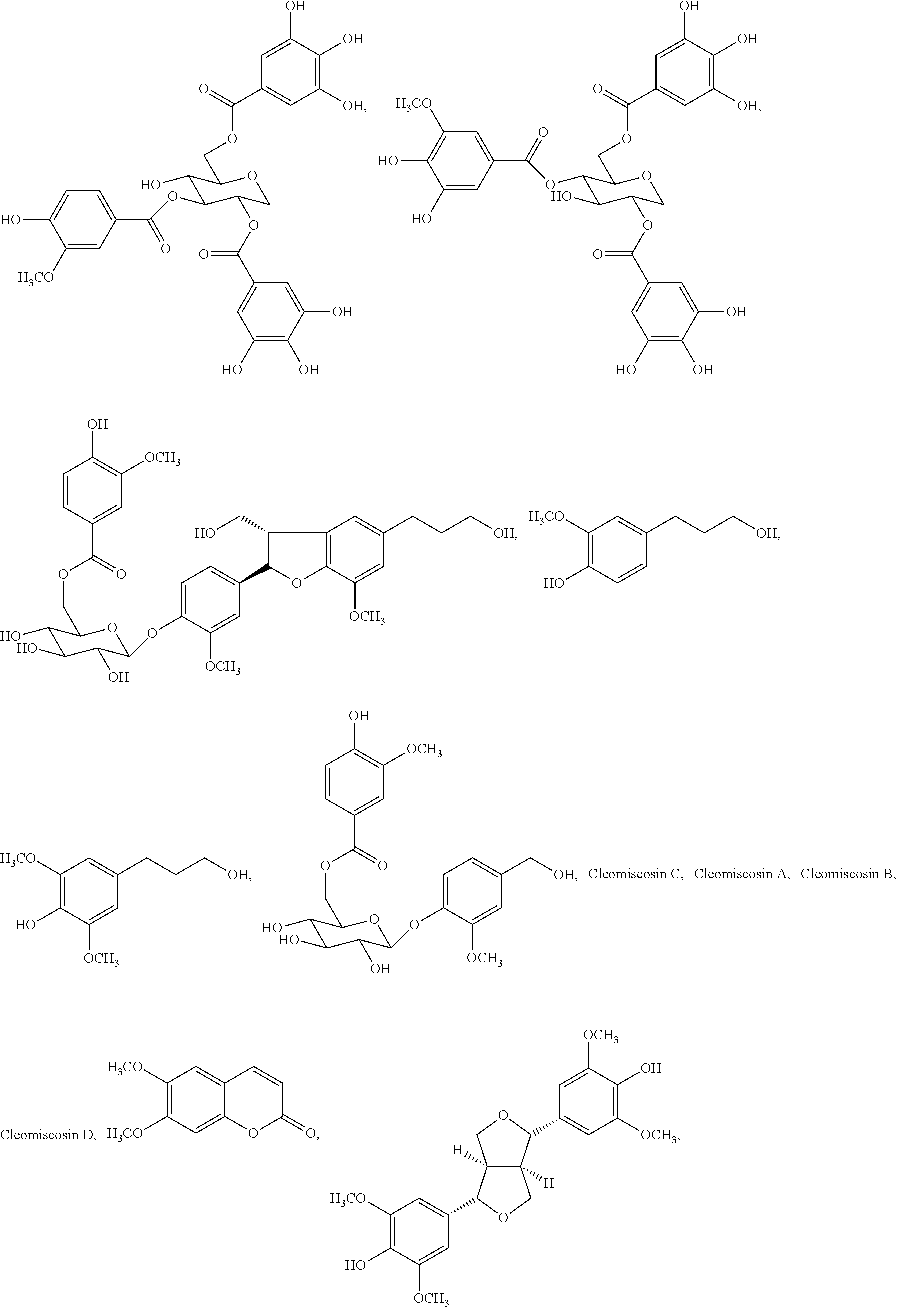

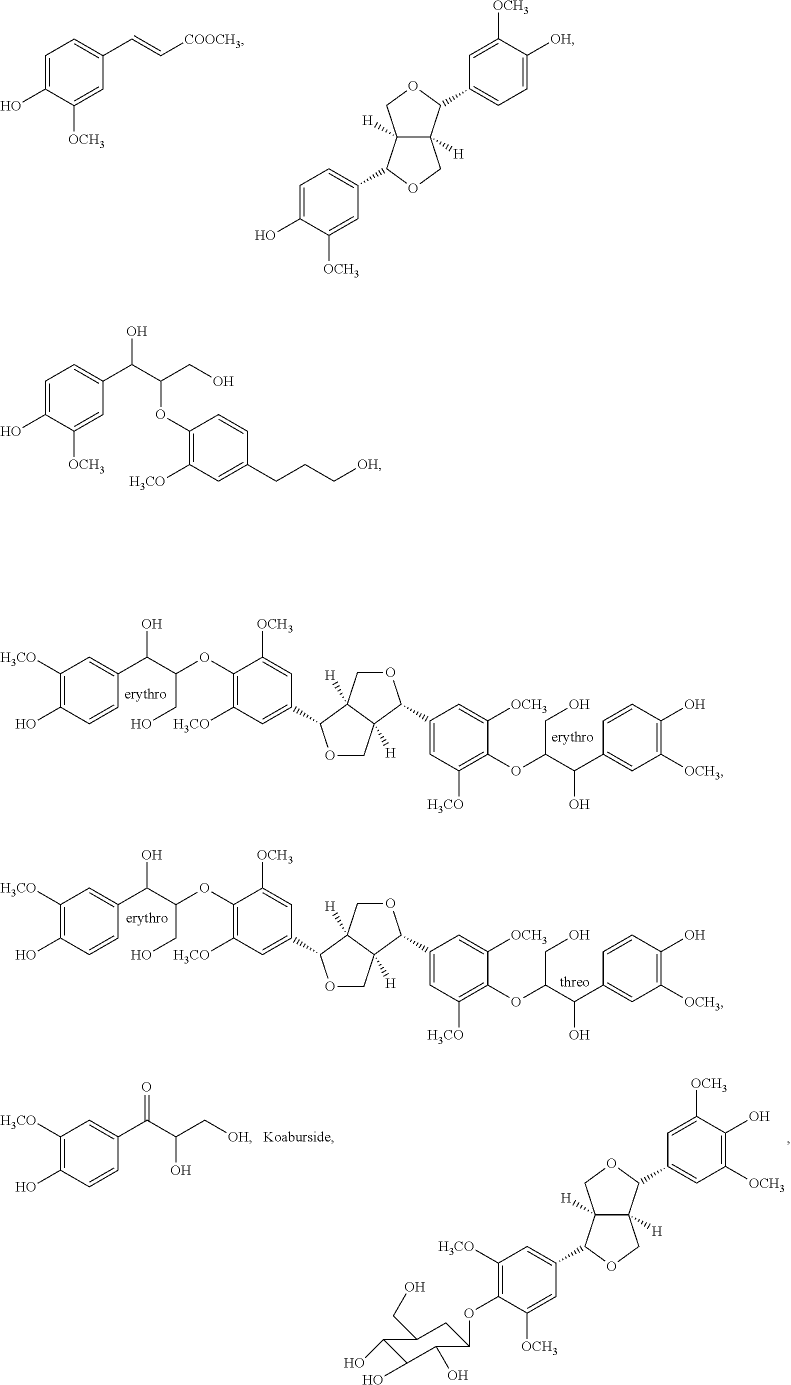

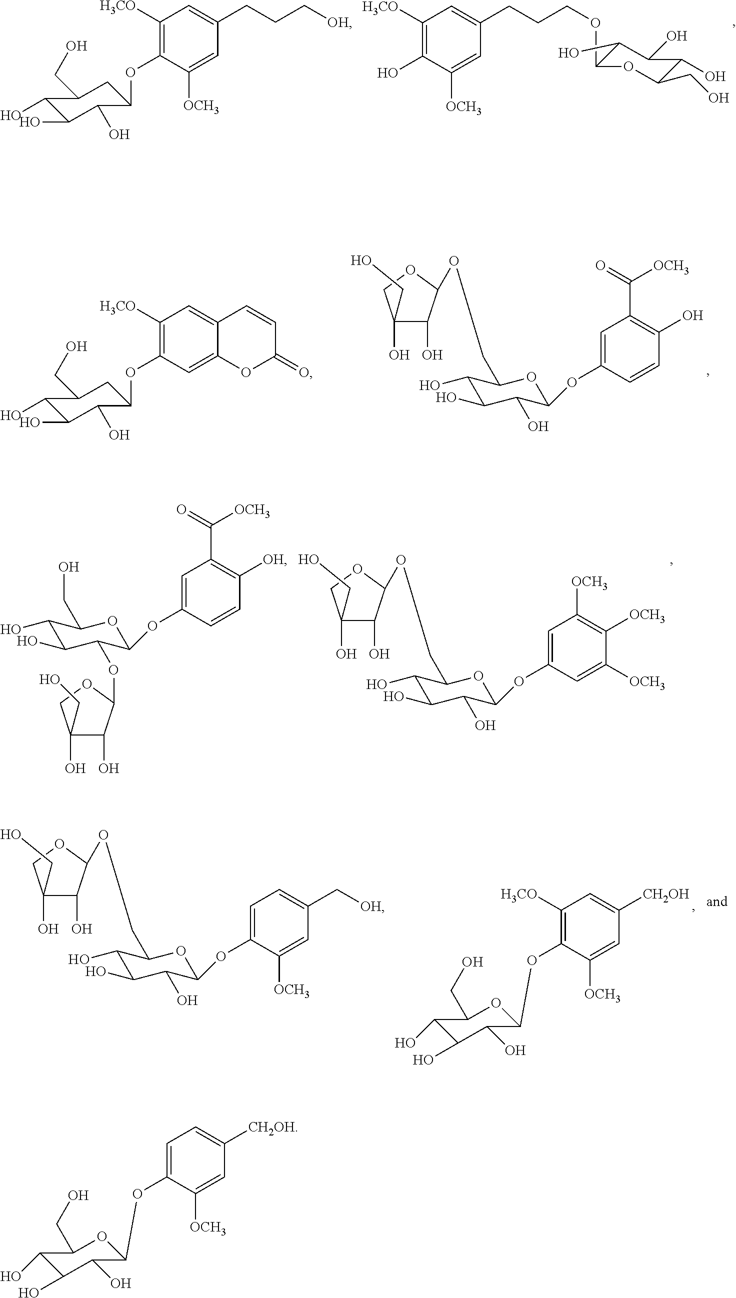

10. The composition according to claim 2, wherein the residue of syrup or syrup-derived product production comprises diatomaceous earth, celite, kieselguhr, silica, silicon dioxide, calcium, natural sugar sand, ground bones, slop, clay and the like.

11. The composition according to any one of claims 1-10, wherein said composition is chosen from a nutraceutical composition, a cosmeceutical composition, a pharmaceutical composition and a functional food composition.

12. A method of prophylaxis and/or treatment of an ailment comprising administering to a subject in need thereof a composition according to any one of claims 1-11.

13. The method of claim 12, wherein said ailment is a diabetes, a cancer, an arthritis, a micro-organism infection, a neurodegenerative disease, an inflammatory disease, an oxidative stress related disease, a heart disease, Alzheimer's diseases, a liver disorder a metabolic syndrome, a damaged hepatic function, a hepatic and liver dyslipidemia, a hepatitis, a liver cancer, an atherosclerosis, a hypertension, a skin disease, an eczema, and a psoriasis.

14. Use of a composition according to any one of claims 1-11 for the prophylaxis and/or treatment of an ailment.

15. The use of claim 14, wherein said ailment is a diabetes, a cancer, an arthritis, a micro-organism infection, a neurodegenerative disease, an inflammatory disease, an oxidative stress related disease, a heart disease, Alzheimer's diseases, a liver disorder a metabolic syndrome, a damaged hepatic function, a hepatic and liver dyslipidemia, a hepatitis, a liver cancer, an atherosclerosis, a hypertension, a skin disease, an eczema, and a psoriasis.

16. An ingredient composition comprising an extract of an Acer tree in association with an acceptable carrier.

17. The ingredient composition according to claim 16, wherein said extract of Acer tree is an extract from a non-concentrated or concentrated sap, a samara fruit, a samara seed, a stems of leaf, a stem of a samara, a twig, a root, a leaf, a bark, a heartwood, a sapwood, a whole branch, a bark of a branch, a wood of a branch, a sugar a syrup, a syrup extract, a syrup-derived product, a rejection of syrup or syrup-derived product production, a residue of syrup or syrup-derived product production, or combinations thereof.

18. The ingredient composition according to claim 16, wherein the syrup derived product comprises butter, granulated sugar, hardened sugar, soft sugar, taffy, flakes, an extract from lyophilisation of a sap, a maple concentrate or a maple syrup, an extract from drying of a sap, a maple concentrate or a maple syrup, an extract from crystallization of a sap, a maple concentrate or a maple syrup, an extract from pulverization of a sap, a maple concentrate or a maple syrup, an extract from atomization of a sap, a maple concentrate or a maple syrup, an extract from centrigugation of a sap, a maple concentrate or a maple syrup or combinations thereof.

19. The ingredient composition according to claim 16, wherein the syrup extract is chosen from a methanol extract, a butanol extract, a butanol extract with sugar, a butanol extract without sugar, an ethyl acetate extract, an ethanol extract, a 95% ethanol/5% hot water extract, or combinations thereof.

20. The ingredient composition according to claim 16, wherein the extract of Acer tree comprises an extract from concentrated Acer tree water issued from reverse osmosis, a concentrated Acer tree water issued from pre-boiling nanofiltration, a pasteurized Acer tree water, a sterilized Acer tree water, an Acer tree water issued from a high pressure processing, an Acer tree water sterilized by UV irradiation, an Acer tree water sterilized by microwave irradiation and combinations thereof.

21. The ingredient composition according to claim 16, wherein the extract of Acer tree comprises an Acer tree molecule.

22. The ingredient composition according to claim 21, wherein the Acer tree molecule comprises:
Lyoniresinol,
Isolariciresinol,
secoisolariciresinol,
Dehydroconiferyl alcohol,
5′-methoxy-dehydroconiferyl alcohol,
erythro-guaiacylglycerol-β-O-4′-coniferyl alcohol,
erythro-guaiacylglycerol-β-O-4′-dihydroconiferyl alcohol,
[3-[4-[(6-deoxy-α-L-mannopyranosyl)oxy]-3-methoxyphenyl]methyl]-5-(3,4-dimethoxyphenyl)dihydro-3-hydroxy-4-(hydroxymethyl)-2(3H)-furanone,
5-(3″,4″-dimethoxyphenyl)-3-hydroxy-3-(4′-hydroxy-3′-methoxybenzyl)-4-hydroxymethyl-dihydrofuran-2-one,
Scopoletin,
Fraxetin,
Isofraxidin,
Gallic acid,
Ginnalin A (acertannin),
Syringic acid,
Ginnalin B,
Ginnalin C,
Trimethyl gallic acid methyl ester
(E)-3,3′-dimethoxy-4,4′-dihydroxy stilbene,
p-coumaric acid,
Ferulic acid,
(E)-Coniferol,
Syringenin,
Dihydroconiferyl alcohol,
C-veratroylglycol,
2,3-dihydroxy-1-(3,4-dihydroxyphenyl)-1-propanone
2,3-Dihydroxy-1-(4-hydroxy-3,5-dimethoxyphenyl)-1-propanone,
3-Hydroxy-1-(4-hydroxy-3,5-dimethoxyphenyl)propan-1-one,
3′,4′,5′-Trihydroxyacetophenone,
4-Acetylcatechol,
2,4,5-Trihydroxyacetophenone,
1-(2,3,4-trihydroxy-5-methylphenyl)-ethanone,
2-Hydroxy-3′,4′-dihydroxyacetophenone,
Vanillin,
Syringaldehyde,
Catechaldehyde,
3,4-Dihydroxy-2-methylbenzaldehyde,
Catechol,
Catechin,
Epicatechin,
Quebecol,
(erythro, etythro)-1-[4-[2-hydroxy-2-(4-hydroxy-3-methoxyphenyl)-1-(hydroxymethyl)ethoxy]-3,5-dimethoxyphenyl]-1,2,3-propanetriol,
(erythro, threo)-1-[4-[2-hydroxy-2-(4-hydroxy-3-methoxyphenyl)-1-(hydroxymethyl)ethoxy]-3,5-dimethoxyphenyl]-1,2,3-propanetriol,
(threo, erythro)-1-[4-[(2-hydroxy-2-(4-hydroxy-3-methoxyphenyl)-1-(hydroxymethyl)ethoxy]-3-methoxyphenyl]-1,2,3-propanetriol,
(threo, threo)-1-[4-[(2-hydroxy-2-(4-hydroxy-3-methoxyphenyl)-1-(hydroxymethyl)ethoxy]-3-methoxyphenyl]-1,2,3-propanetriol,
threo-guaiacylglycerol-β-O-4′-dihydroconiferyl alcohol,
erythro-1-(4-hydroxy-3-methoxyphenyl)-2-[4-(3-hydroxypropyl)-2,6-dimethoxyphenoxy]-1,3-propanediol,
2-[4-[2,3-dihydro-3-(hydroxymethyl)-5-(3-hydroxypropyl)-7-methoxy-2-benzofuranyl]-2,6-dimethoxyphenoxy]-1-(4-hydroxy-3-methoxyphenyl)-1,3-propanediol,
Acerkinol,
Leptolepisol D,
Buddlenol E,
(1S,2R)-2-[2,6-dimethoxy-4-[(1S,3aR,4S,6aR)-tetrahydro-4-(4-hydroxy-3,5-dimethoxyphenyl)-1H,3H-furo[3,4-c]furan-1-yl]phenoxy]-1-(4-hydroxy-3-methoxyphenyl)-1,3-propanediol,
Syringaresinol,
Icariside E4,
Sakuraresinol,
1,2-diguaiacyl-1,3-propanediol
protocatechuic acid,
4-(dimethoxymethyl)-pyrocatechol,
Tyrosol,
4-hydroxycatechol,
Phaseic acid,
Nortrachelogenin 8′-O-3-D-glucopyranoside,
3-O-galloyl-1,5-anhydro-D-glucitol,
4-O-galloyl-1,5-anhydro-D-glucitol,
2,4-di-O-galloyl-1,5-anhydro-D-glucitol,
Quercetin-3-O-α-rhamnopyranoside,
2,3-di-O-galloyl-1,5-anhydro-D-glucitol,
3-Methoxy-4-hydroxyphenol 1-O-β-D-(6′-O-galloyl)-glucopyranoside,
3,5-Dihydroxy-4-methoxybenzoic acid methyl ester,
7,8-Dihydroxy-6-methoxycoumarin,
4-Methoxy-3,5-dihydroxybenzoic acid,
Methyl 3,5-dimethoxy-4-hydroxybenzoate,
Methyl vanillate,
Methyl gallate,
3,6-di-O-galloyl-1,5-anhydro-D-glucitol,
(+)-Epicatechin-(4β-8,2β-O-7)-catechin,
Epicatechin gallate,
(+)-Epicatechin-(4β-8,2β-O-7)-epicatechin,
Quercetin-3-O-(3″-O-galloyl)-α-rhamnopyranoside,
Quercetin-3-O-(2″-O-galloyl)-α-rhamnopyranoside,
2,4,6-tri-O-galloyl-1,5-anhydro-D-glucitol,

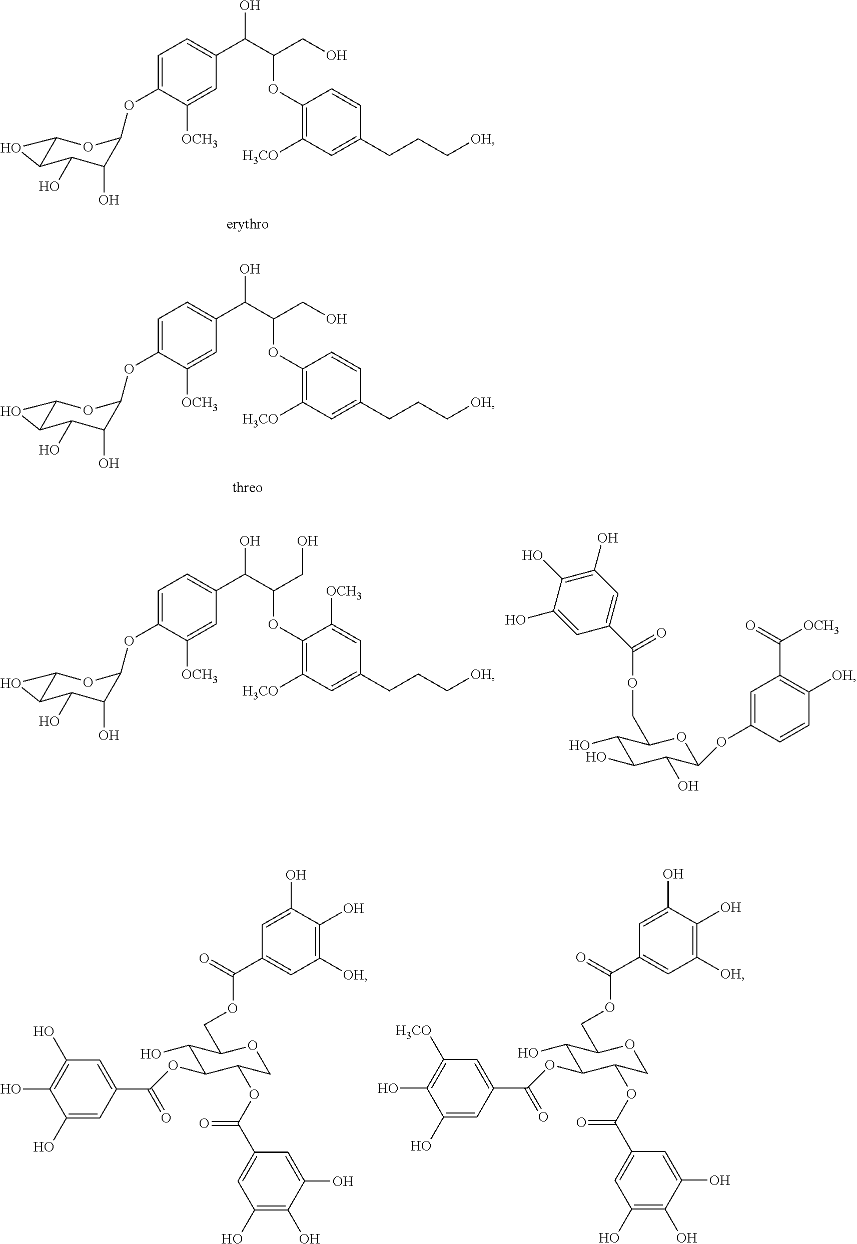 [SMILES: COc1cc(CCCO)ccc1OC(CO)C(O)c1ccc(OC2OC3OC3C(O)C2O)c(OC)c1 ; COc1cc(CCCO)ccc1OC(CO)C(O)c1ccc(OC2OC3OC3C(O)C2O)c(OC)c1 ; COc1cc(C(O)C(CO)Oc2c(OC)cc(CCCO)cc2OC)ccc1OC1OC2OC2C(O)C1O ; COC(=O)c1cc(OC2OC(COC(=O)c3cc(O)c(O)c(O)c3)C(O)C(O)C2O)ccc1O ; O=C(OCC1OCC(OC(=O)c2cc(O)c(O)c(O)c2)C(OC(=O)c2cc(O)c(O)c(O)c2)C1O)c1cc(O)c(O)c(O)c1 ; COc1cc(C(=O)OC2C(OC(=O)c3cc(O)c(O)c(O)c3)COC(COC(=O)c3cc(O)c(O)c(O)c3)C2O)cc(O)c1O]

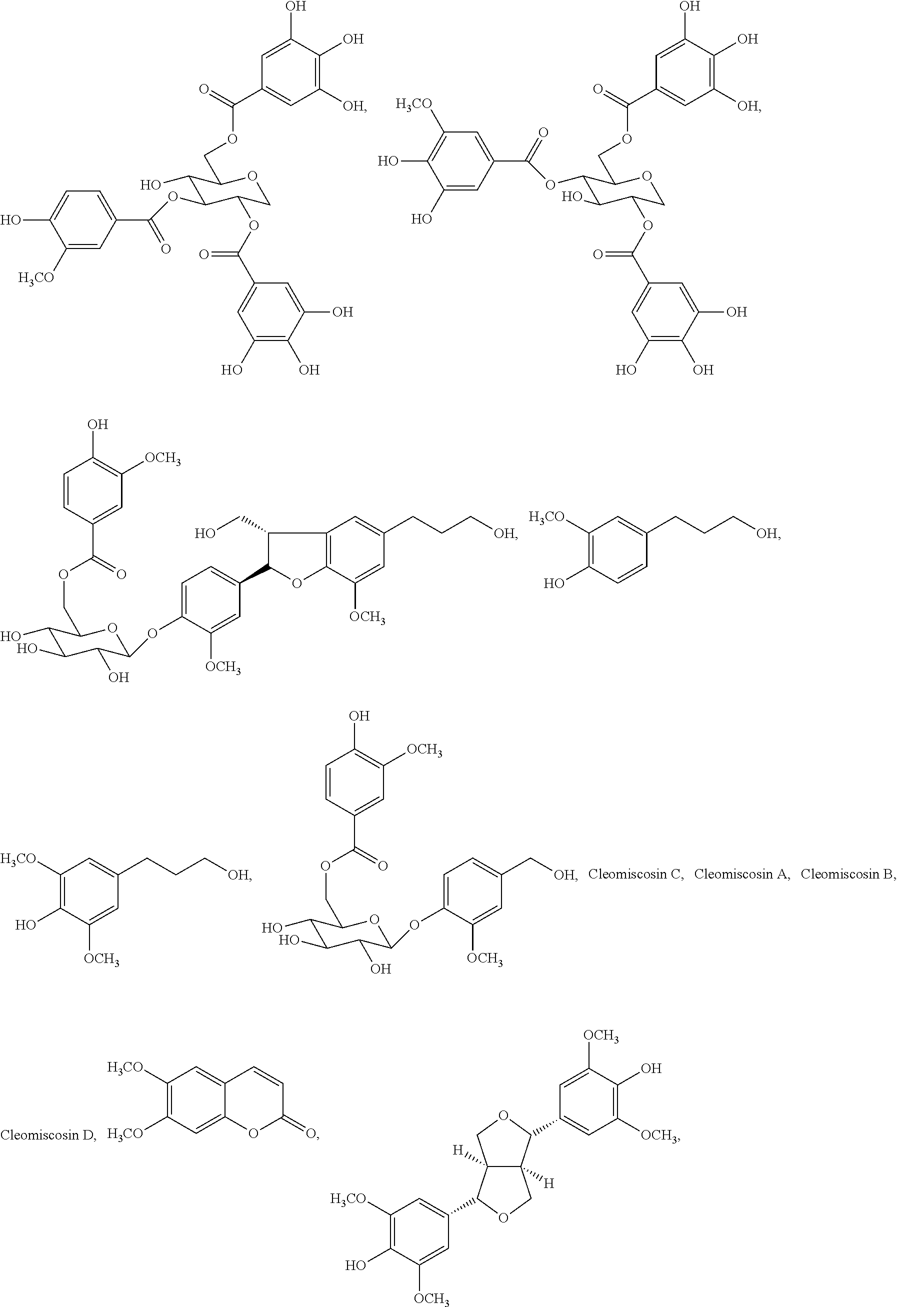

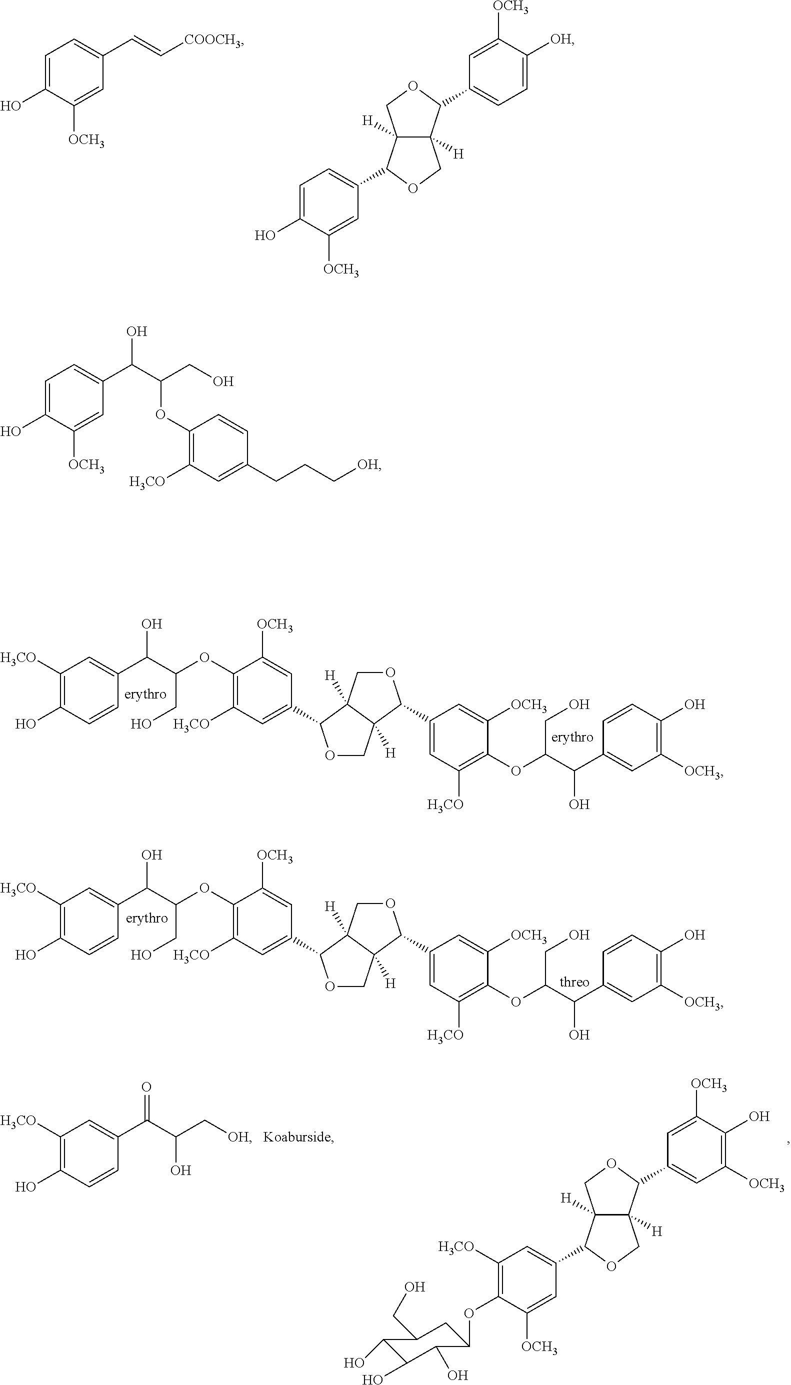

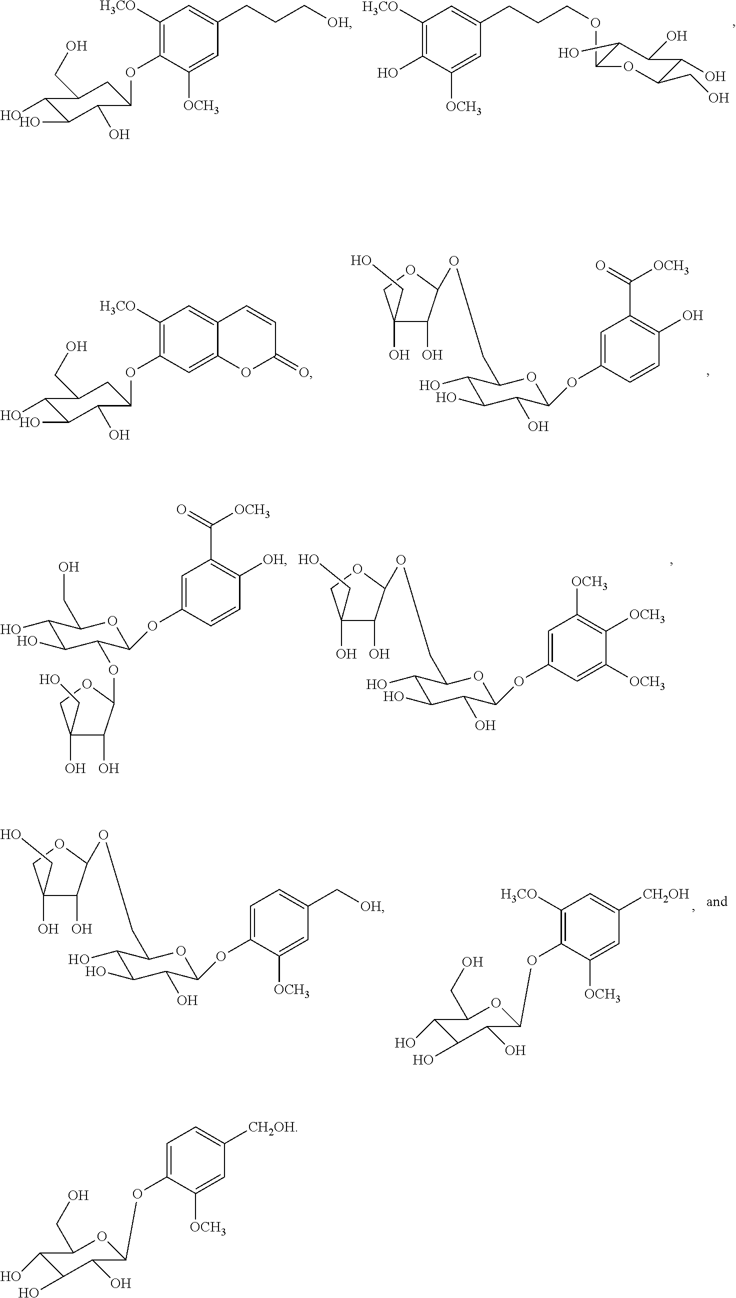

23. The ingredient composition according to claim 17, wherein the residue of syrup or syrup-derived product production comprises diatomaceous earth, celite, kieselguhr, silica, silicon dioxide, calcium, natural sugar sand, ground bones, slop, clay and the like.

24. The ingredient composition according to any one of claims 16-23, wherein said Acer tree is chosen from Acer nigrum, Acer lanum, Acer acuminatum, Acer albopurpurascens, Acer argutum, Acer barbinerve, Acer buergerianum, Acer caesium, Acer campbellii, Acer campestre, Acer capillipes, Acer cappadocicum, Acer carpinifolium, Acer caudatifolium, Acer caudatum, Acer cinnamomifolium, Acer circinatum, Acer cissifolium, Acer crassum, Acer crataegifolium, Acer davidii, Acer decandrum, Acer diabolicum, Acer distylum, Acer divergens, Acer erianthum, Acer erythranthum, Acer fabri, Acer garrettii, Acer glabrum, Acer grandidentatum, Acer griseum, Acer heldreichii, Acer henryi, Acer hyrcanum, Acer ibericum, Acer japonicum, Acer kungshanense, Acer kweilinense, Acer laevigatum, Acer laurinum, Acer lobelii, Acer lucidum, Acer macrophyllum, Acer mandshuricum, Acer maximowiczianum, Acer miaoshanicum, Acer micranthum, Acer miyabei, Acer mono, Acer mono×Acer truncatum, Acer monspessulanum, Acer negundo, Acer ningpoense, Acer nipponicum, Acer oblongum, Acer obtusifolium, Acer oliverianum, Acer opalus, Acer palmatum, Acer paxii, Acer pectinatum, Acer pensylvanicum, Acer pentaphyllum, Acer pentapomicum, Acer pictum, Acer pilosum, Acer platanoides, Acer poliophyllum, Acer pseudoplatanus, Acer pseudosieboldianum, Acer pubinerve, Acer pycnanthum, Acer rubrum, Acer rufinerve, Acer saccharinum, Acer saccharum, Acer sempervirens, Acer shirasawanum, Acer sieboldianum, Acer sinopurpurescens, Acer spicatum, Acer stachyophyllum, Acer sterculiaceum, Acer takesimense, Acer tataricum, Acer tegmentosum, Acer tenuifolium, Acer tetramerum, Acer trautvetteri, Acer triflorum, Acer truncatum, Acer tschonoskii, Acer turcomanicum, Acer ukurunduense, Acer velutinum, Acer wardii, Acer x peronai, and Acer x pseudoheldreichii.

25. The ingredient composition according to any one of claims 16-23, wherein said Acer tree is chosen from Acer Saccharum and Acer Rubrum L.

26. The ingredient composition according to any one of claims 16-25, wherein said ingredient composition a food ingredient composition, a non-food ingredient composition, or combination thereof.

27. A method of seasoning a food comprising administering to a food an ingredient composition according to any one of claims 16-26.

28. An Acer tree essential oil composition comprising
a hydrophobic fraction extracted from an Acer tree biomass; and
a suitable solvent.

29. The essential oil composition according to claim 28, wherein said Acer tree biomass is from at least one of a samara fruit, a samara seed, a stem of leaf, a stem of a samara, a twig, a root, a leaf, a bark, a heartwood, a sapwood, a whole branch, a bark of a branch, a wood of a branch.

30. The essential oil composition according to claim 28, wherein the hydrophobic fraction extracted from an Acer tree biomass comprises an Acer tree molecule.

31. The essential oil composition according to claim 65, wherein the Acer tree molecule comprises:
Lyoniresinol,
Isolariciresinol,
secoisolariciresinol,
Dehydroconiferyl alcohol,
5′-methoxy-dehydroconiferyl alcohol,
erythro-guaiacylglycerol-[3-O-4′-coniferyl alcohol,
erythro-guaiacylglycerol-β-O-4′-dihydroconiferyl alcohol,
[3-[4-[(6-deoxy-α-L-mannopyranosyl)oxy]-3-methoxyphenyl]methyl]-5-(3,4-dimethoxyphenyl)dihydro-3-hydroxy-4-(hydroxymethyl)-2(3H)-furanone,
5-(3″,4″-dimethoxyphenyl)-3-hydroxy-3-(4′-hydroxy-3′-methoxybenzyl)-4-hydroxymethyl-dihydrofuran-2-one,
Scopoletin,
Fraxetin,
Isofraxidin,
Gallic acid,
Ginnalin A (acertannin),
Syringic acid,
Ginnalin B,
Ginnalin C,
Trimethyl gallic acid methyl ester
(E)-3,3′-dimethoxy-4,4′-dihydroxy stilbene,
p-coumaric acid,
Ferulic acid,
(E)-Coniferol,
Syringenin,
Dihydroconiferyl alcohol,
C-veratroylglycol,
2,3-dihydroxy-1-(3,4-dihydroxyphenyl)-1-propanone
2,3-Dihydroxy-1-(4-hydroxy-3,5-dimethoxyphenyl)-1-propanone,
3-Hydroxy-1-(4-hydroxy-3,5-dimethoxyphenyl)propan-1-one,
3′,4′,5′-Trihydroxyacetophenone,
4-Acetylcatechol,
2,4,5-Trihydroxyacetophenone,
1-(2,3,4-trihydroxy-5-methylphenyl)-ethanone,
2-Hydroxy-3′,4′-dihydroxyacetophenone,
Vanillin,
Syringaldehyde,
Catechaldehyde,
3,4-Dihydroxy-2-methylbenzaldehyde,
Catechol,
Catechin,
Epicatechin,
Quebecol,
(erythro, erythro)-1-[4-[2-hydroxy-2-(4-hydroxy-3-methoxyphenyl)-1-(hydroxymethyl)ethoxy]-3,5-dimethoxyphenyl]-1,2,3-propanetriol,
(erythro, threo)-1-[4-[2-hydroxy-2-(4-hydroxy-3-methoxyphenyl)-1-(hydroxymethyl)ethoxy]-3,5-dimethoxyphenyl]-1,2,3-propanetriol,
(threo, erythro)-1-[4-[(2-hydroxy-2-(4-hydroxy-3-methoxyphenyl)-1-(hydroxymethyl)ethoxy]-3-methoxyphenyl]-1,2,3-propanetriol,
(threo, threo)-1-[4-[(2-hydroxy-2-(4-hydroxy-3-methoxyphenyl)-1-(hydroxymethyl)ethoxy]-3-methoxyphenyl]-1,2,3-propanetriol,
threo-guaiacylglycerol-β-O-4′-dihydroconiferyl alcohol,
erythro-1-(4-hydroxy-3-methoxyphenyl)-2-[4-(3-hydroxypropyl)-2,6-dimethoxyphenoxy]-1,3-propanediol,
2-[4-[2,3-dihydro-3-(hydroxymethyl)-5-(3-hydroxypropyl)-7-methoxy-2-benzofuranyl]-2,6-dimethoxyphenoxy]-1-(4-hydroxy-3-methoxyphenyl)-1,3-propanediol,
Acerkinol,
Leptolepisol D,
Buddlenol E,
(1S,2R)-2-[2,6-dimethoxy-4-[(1S,3aR,4S,6aR)-tetrahydro-4-(4-hydroxy-3,5-dimethoxyphenyl)-1H,3H-furo[3,4-c]furan-1-yl]phenoxy]-1-(4-hydroxy-3-methoxyphenyl)-1,3-propanediol,
Syringaresinol,
Icariside E4,
Sakuraresinol,
1,2-diguaiacyl-1,3-propanediol
protocatechuic acid,
4-(dimethoxymethyl)-pyrocatechol,
Tyrosol,
4-hydroxycatechol,
Phaseic acid,
Nortrachelogenin 8′-O-β-D-glucopyranoside,
3-O-galloyl-1,5-anhydro-D-glucitol,
4-O-galloyl-1,5-anhydro-D-glucitol,
2,4-di-O-galloyl-1,5-anhydro-D-glucitol,
Quercetin-3-O-α-rhamnopyranoside,
2,3-di-O-galloyl-1,5-anhydro-D-glucitol,
3-Methoxy-4-hydroxyphenol 1-O-β-D-(6′-O-galloyl)-glucopyranoside,
3,5-Dihydroxy-4-methoxybenzoic acid methyl ester,
7,8-Dihydroxy-6-methoxycoumarin,
4-Methoxy-3,5-dihydroxybenzoic acid,
Methyl 3,5-dimethoxy-4-hydroxybenzoate,
Methyl vanillate,
Methyl gallate,
3,6-di-O-galloyl-1,5-anhydro-D-glucitol,
(+)-Epicatechin-(4β-8,2β-O-7)-catechin,
Epicatechin gallate,
(+)-Epicatechin-(4β-8,2β-O-7)-epicatechin,
Quercetin-3-O-(3″-O-galloyl)-α-rhamnopyranoside,
Quercetin-3-O-(2″-O-galloyl)-α-rhamnopyranoside,
2,4,6-tri-O-galloyl-1,5-anhydro-D-glucitol,

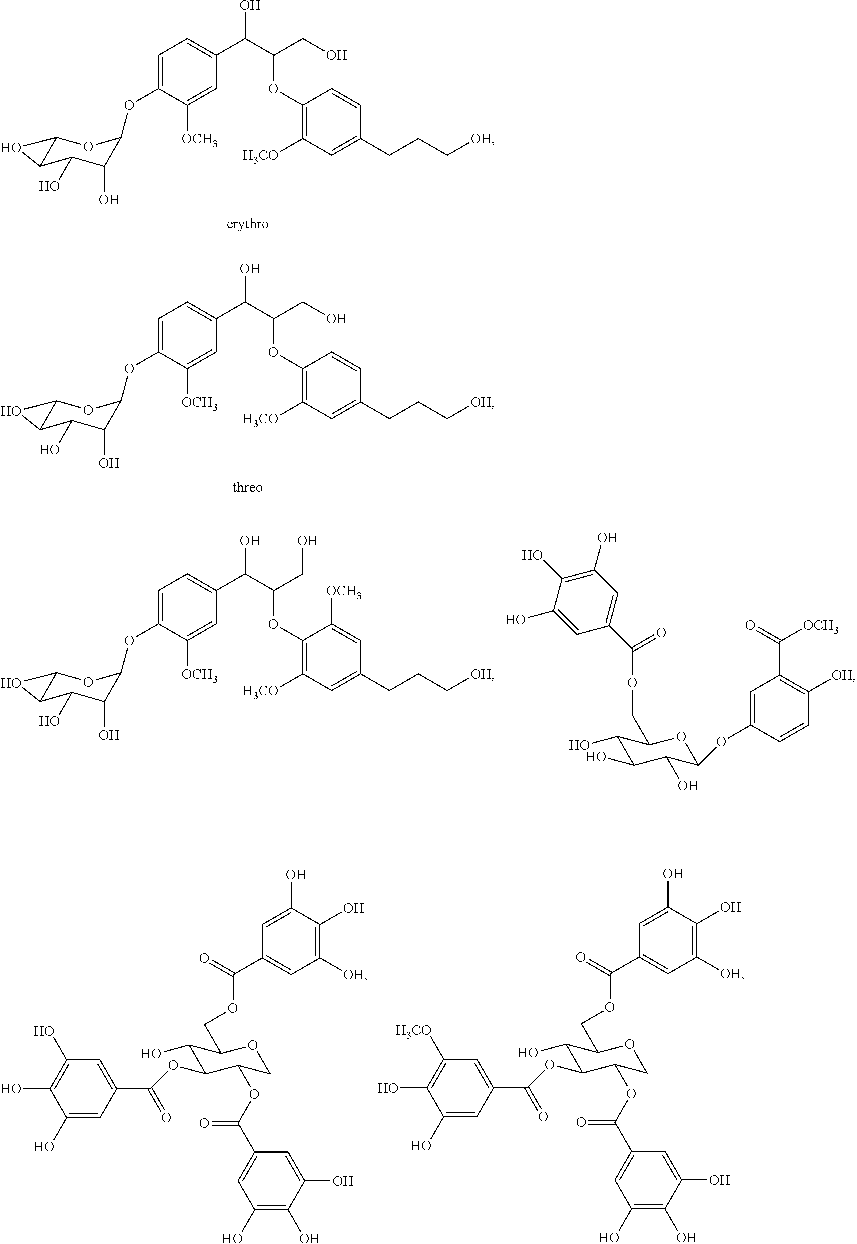 [SMILES: COc1cc(CCCO)ccc1OC(CO)C(O)c1ccc(OC2OC3OC3C(O)C2O)c(OC)c1 ; COc1cc(CCCO)ccc1OC(CO)C(O)c1ccc(OC2OC3OC3C(O)C2O)c(OC)c1 ; COc1cc(C(O)C(CO)Oc2c(OC)cc(CCCO)cc2OC)ccc1OC1OC2OC2C(O)C1O ; COC(=O)c1cc(OC2OC(COC(=O)c3cc(O)c(O)c(O)c3)C(O)C(O)C2O)ccc1O ; O=C(OCC1OCC(OC(=O)c2cc(O)c(O)c(O)c2)C(OC(=O)c2cc(O)c(O)c(O)c2)C1O)c1cc(O)c(O)c(O)c1 ; COc1cc(C(=O)OC2C(OC(=O)c3cc(O)c(O)c(O)c3)COC(COC(=O)c3cc(O)c(O)c(O)c3)C2O)cc(O)c1O]

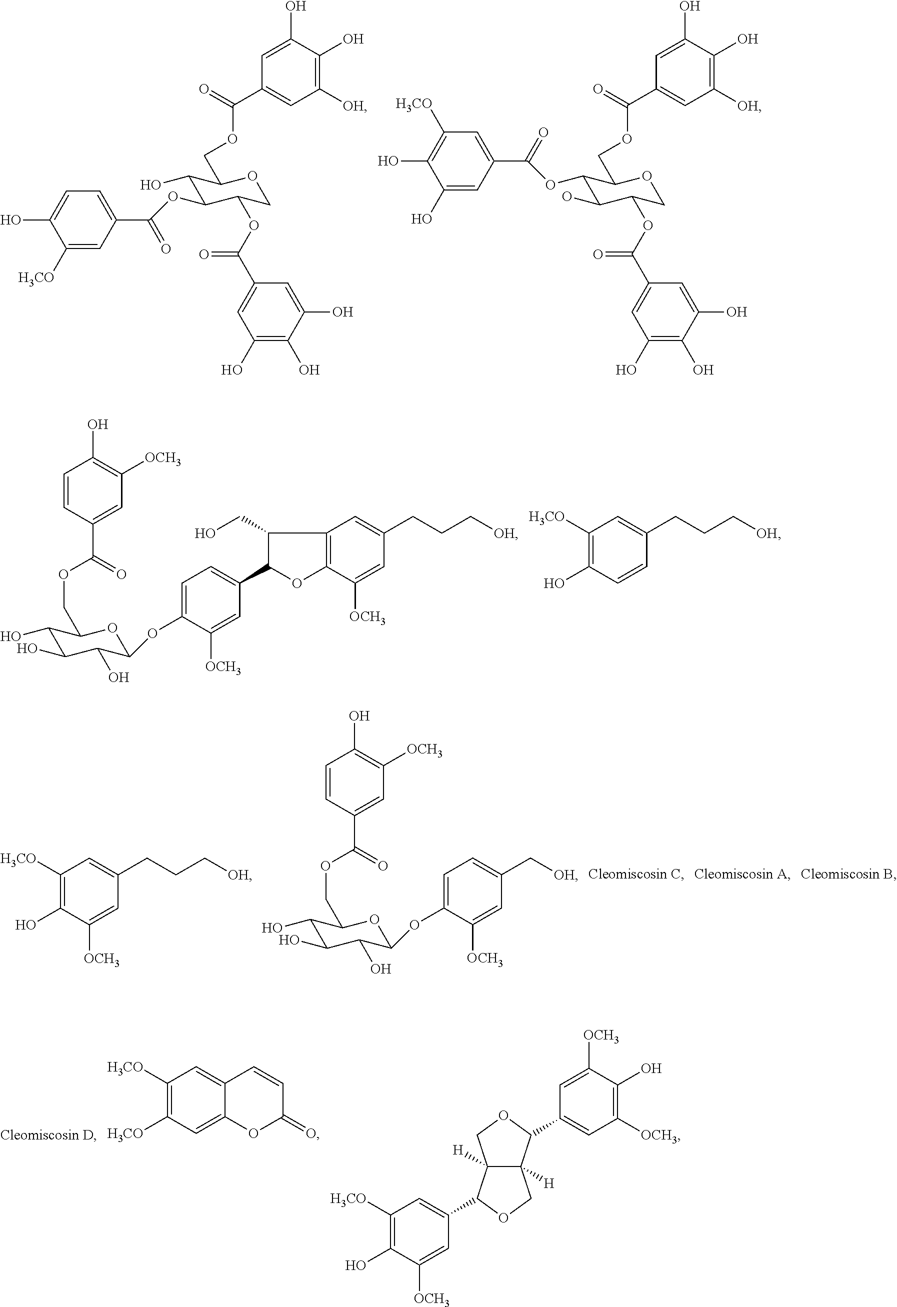

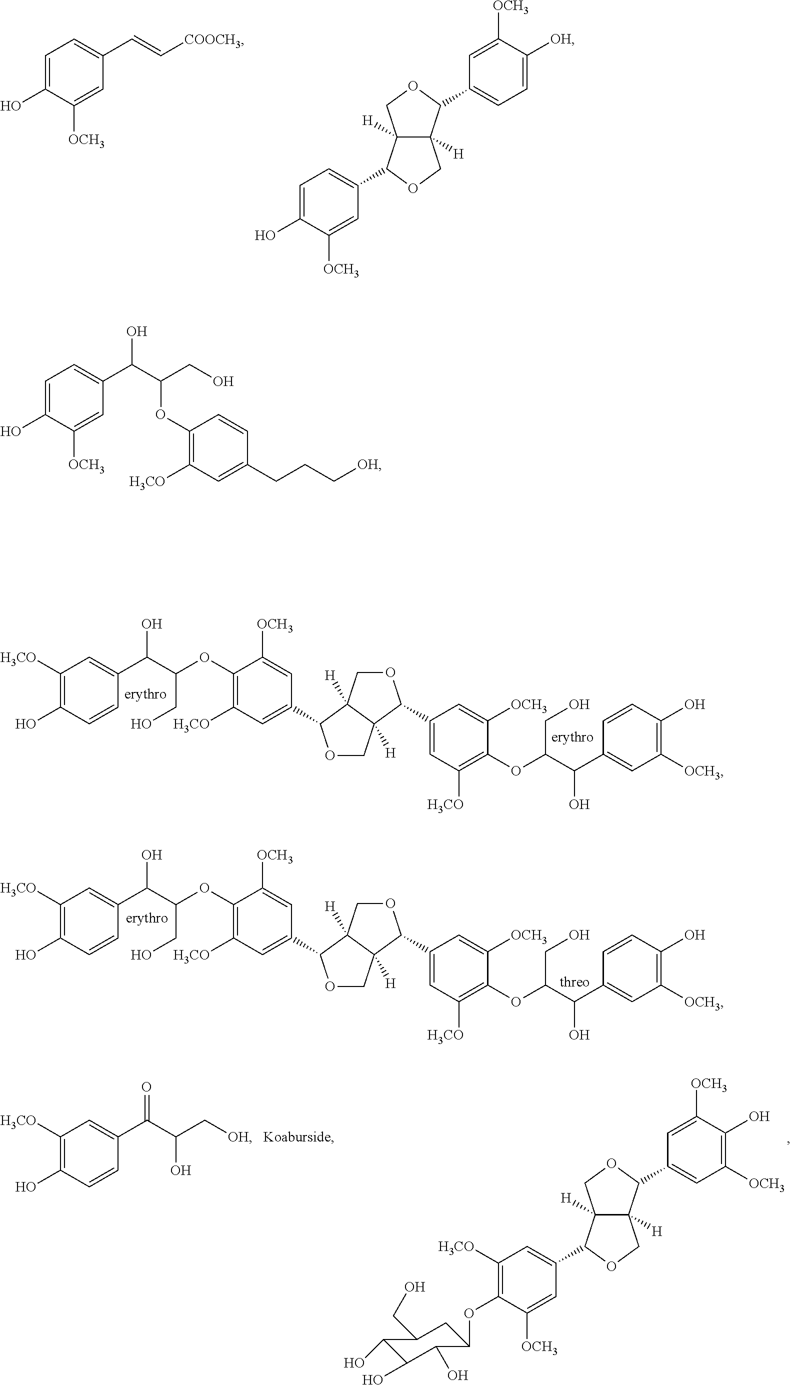

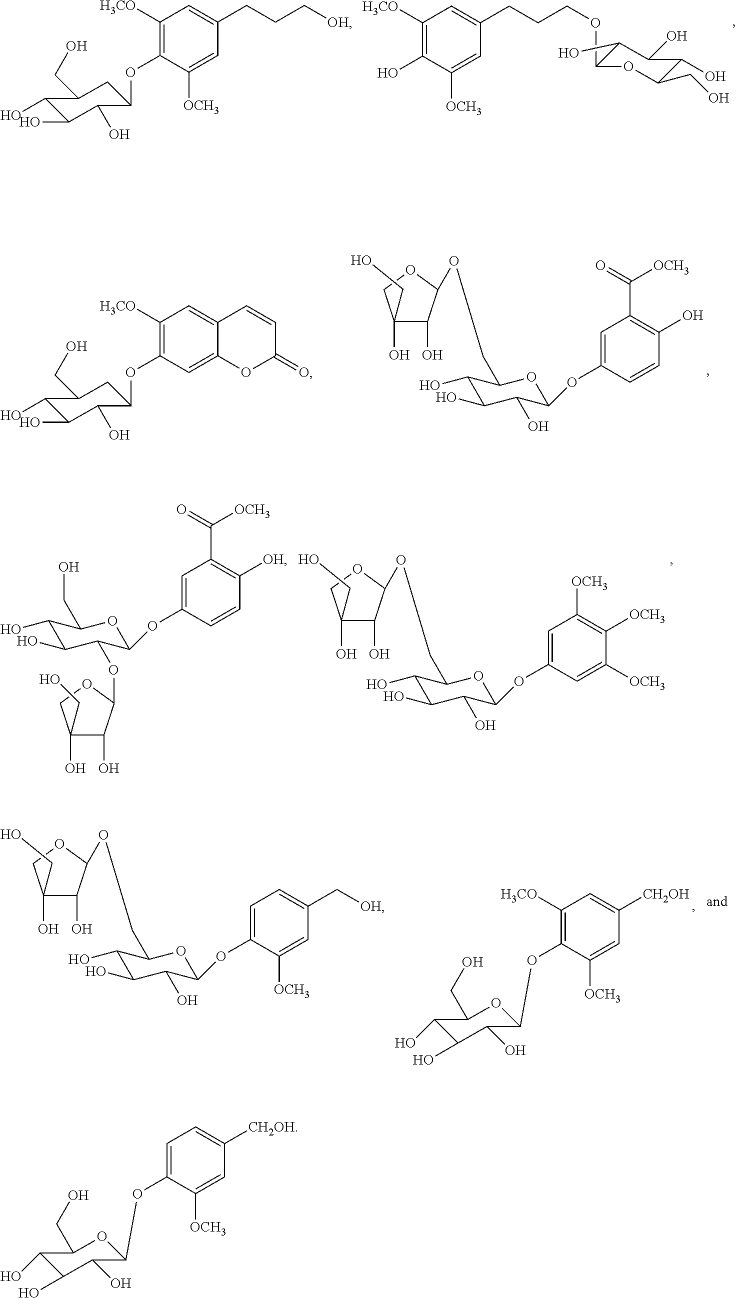

32. The essential oil composition according to any one of claims 28-31, wherein said suitable solvent is at least one of ethanol, polyethylene glycol, or a pharmaceutically acceptable carrier oil.

33. The essential oil composition according to claim 32, wherein said pharmaceutically acceptable carrier oil is at least one of sweet almond oil, kukui nut oil, apricot kernel oil, macadamia nut oil, avocado oil, meadowfoam oil, borage seed oil, olive oil, camellia seed oil, peanut oil, cranberry seed oil, pecan oil, evening primrose oil, pomegranate seed oil, fractionated coconut oil, rose hip oil, grapeseed oil, seabuckthorn berry oil, hazelnut oil, sesame oil, hemp seed oil, sunflower oil, jojoba, and watermelon seed oil.

34. A method of preparing a maple syrup comprising adding a hydrophobic fraction extracted from an Acer tree biomass before or during the preparation of a maple syrup.

35. A food composition comprising:
a plurality of Acer tree samara.

36. The food composition according to claim 35, wherein said Acer tree samara is germinated.

37. The food composition according to claim 35, wherein said Acer tree samara comprises a fruit.

38. The food composition according to claim 35, wherein said Acer tree samara comprises a seed.

39. The food composition according to claim 35, wherein said Acer tree samara is dried and/or fermented.

40. The food composition according to claim 35, wherein said Acer tree samara is dessicated.

41. The food composition according to claim 35, wherein said germinated Acer tree samara is marinated.

42. The food composition according to any one of claims 35-41, further comprising a seasoning ingredient chosen from a salt, a pepper, a cheese, an oil, a vinegar, a salad sauce, and a vinaigrette.

43. The food composition according to claim 42, wherein said salt is chosen from sodium chloride, a sea salt, and sodium acetate.

44. A solid sweetening composition for oral consumption comprising:
a Acer tree sugar extract;
at least one sweetener, and
a dietary acceptable filler.

45. The sweetening composition according to claim 44, wherein said Acer tree sugar extract is at least one of non-concentrated or concentrated sap, syrup, a syrup, a syrup extract, a syrup-derived product, a rejection of syrup or syrup-derived product production, a residue of syrup or syrup-derived product production, taffy, flakes, sugar, spread, an extract from lyophilisation of a sap, a maple concentrate or a maple syrup, an extract from drying of a sap, a maple concentrate or a maple syrup, an extract from crystallization of a sap, a maple concentrate or a maple syrup, an extract from pulverization of a sap, a maple concentrate or a maple syrup, an extract from atomization of a sap, a maple concentrate or a maple syrup, an extract from centrigugation of a sap, a maple concentrate or a maple syrup or combinations thereof.

46. The sweetening composition according to claim 45, wherein the syrup derived products comprise butter, granulated sugar, hardened sugar, soft sugar, taffy, flakes, or combinations thereof.

47. The sweetening composition according to claim 45, wherein the syrup extracts is chosen from a methanol extract, a butanol extract, a butanol extract with sugar, a butanol extract without sugar, an ethyl acetate extract, an ethanol extract, a 95% ethanol/5% hot water extract, or combinations thereof.

48. The sweetening composition according to claim 44, wherein the Acer tree sugar extract comprises an Acer tree molecule.

49. The sweetening composition according to claim 48, wherein the Acer tree molecule comprises:
Lyoniresinol,
Isolariciresinol,
secoisolariciresinol,
Dehydroconiferyl alcohol,
5′-methoxy-dehydroconiferyl alcohol,
erythro-guaiacylglycerol-β-O-4′-coniferyl alcohol,
erythro-guaiacylglycerol-β-O-4′-dihydroconiferyl alcohol,
[3-[4-[(6-deoxy-α-L-mannopyranosyl)oxy]-3-methoxyphenyl]methyl]-5-(3,4-dimethoxyphenyl)dihydro-3-hydroxy-4-(hydroxymethyl)-2(3H)-furanone,
5-(3″,4″-dimethoxyphenyl)-3-hydroxy-3-(4′-hydroxy-3′-methoxybenzyl)-4-hydroxymethyl-dihydrofuran-2-one,
Scopoletin,
Fraxetin,
Isofraxidin,
Gallic acid,
Ginnalin A (acertannin),
Syringic acid,
Ginnalin B,
Ginnalin C,
Trimethyl gallic acid methyl ester
(E)-3,3′-dimethoxy-4,4′-dihydroxy stilbene,
p-coumaric acid,
Ferulic acid,
(E)-Coniferol,
Syringenin,
Dihydroconiferyl alcohol,
C-veratroylglycol,
2,3-dihydroxy-1-(3,4-dihydroxyphenyl)-1-propanone
2,3-Dihydroxy-1-(4-hydroxy-3,5-dimethoxyphenyl)-1-propanone,
3-Hydroxy-1-(4-hydroxy-3,5-dimethoxyphenyl)propan-1-one,
3′,4′,5′-Trihydroxyacetophenone,
4-Acetylcatechol,
2,4,5-Trihydroxyacetophenone,
1-(2,3,4-trihydroxy-5-methylphenyl)-ethanone,
2-Hydroxy-3′,4′-dihydroxyacetophenone,
Vanillin,
Syringaldehyde,
Catechaldehyde,
3,4-Dihydroxy-2-methylbenzaldehyde,
Catechol,
Catechin,
Epicatechin,
Quebecol,
(erythro, erythro)-1-[4-[2-hydroxy-2-(4-hydroxy-3-methoxyphenyl)-1-(hydroxymethyl)ethoxy]-3,5-dimethoxyphenyl]-1,2,3-propanetriol,
(erythro, threo)-1-[4-[2-hydroxy-2-(4-hydroxy-3-methoxyphenyl)-1-(hydroxymethyl)ethoxy]-3,5-dimethoxyphenyl]-1,2,3-propanetriol,
(threo, erythro)-1-[4-[(2-hydroxy-2-(4-hydroxy-3-methoxyphenyl)-1-(hydroxymethyl)ethoxy]-3-methoxyphenyl]-1,2,3-propanetriol,
(threo, threo)-1-[4-[(2-hydroxy-2-(4-hydroxy-3-methoxyphenyl)-1-(hydroxymethyl)ethoxy]-3-methoxyphenyl]-1,2,3-propanetriol,
threo-guaiacylglycerol-3-O-4′-dihydroconiferyl alcohol,
erythro-1-(4-hydroxy-3-methoxyphenyl)-2-[4-(3-hydroxypropyl)-2,6-dimethoxyphenoxy]-1,3-propanediol,
2-[4-[2,3-dihydro-3-(hydroxymethyl)-5-(3-hydroxypropyl)-7-methoxy-2-benzofuranyl]-2,6-dimethoxyphenoxy]-1-(4-hydroxy-3-methoxyphenyl)-1,3-propanediol,
Acerkinol,
Leptolepisol D,
Buddlenol E,
(1S,2R)-2-[2,6-dimethoxy-4-[(1S,3aR,4S,6aR)-tetrahydro-4-(4-hydroxy-3,5-dimethoxyphenyl)-1H,3H-furo[3,4-c]furan-1-yl]phenoxy]-1-(4-hydroxy-3-methoxyphenyl)-1,3-propanediol,
Syringaresinol,
Icariside E4,
Sakuraresinol,
1,2-diguaiacyl-1,3-propanediol
protocatechuic acid,
4-(dimethoxymethyl)-pyrocatechol,
Tyrosol,
4-hydroxycatechol,
Phaseic acid,
Nortrachelogenin 8′-O-β-D-glucopyranoside,
3-O-galloyl-1,5-anhydro-D-glucitol,
4-O-galloyl-1,5-anhydro-D-glucitol,
2,4-di-O-galloyl-1,5-anhydro-D-glucitol,
Quercetin-3-O-α-rhamnopyranoside,
2,3-di-O-galloyl-1,5-anhydro-D-glucitol,
3-Methoxy-4-hydroxyphenol 1-O-β-D-(6′-O-galloyl)-glucopyranoside,
3,5-Dihydroxy-4-methoxybenzoic acid methyl ester,
7,8-Dihydroxy-6-methoxycoumarin,
4-Methoxy-3,5-dihydroxybenzoic acid,
Methyl 3,5-dimethoxy-4-hydroxybenzoate,
Methyl vanillate,
Methyl gallate,
3,6-di-O-galloyl-1,5-anhydro-D-glucitol,
(+)-Epicatechin-(46-8,2(3-O-7)-catechin,
Epicatechin gallate,
(+)-Epicatechin-(46-8,2(3-O-7)-epicatechin,
Quercetin-3-O-(3″-O-galloyl)-α-rhamnopyranoside,
Quercetin-3-O-(2″-O-galloyl)-α-rhamnopyranoside,
2,4,6-tri-O-galloyl-1,5-anhydro-D-glucitol,

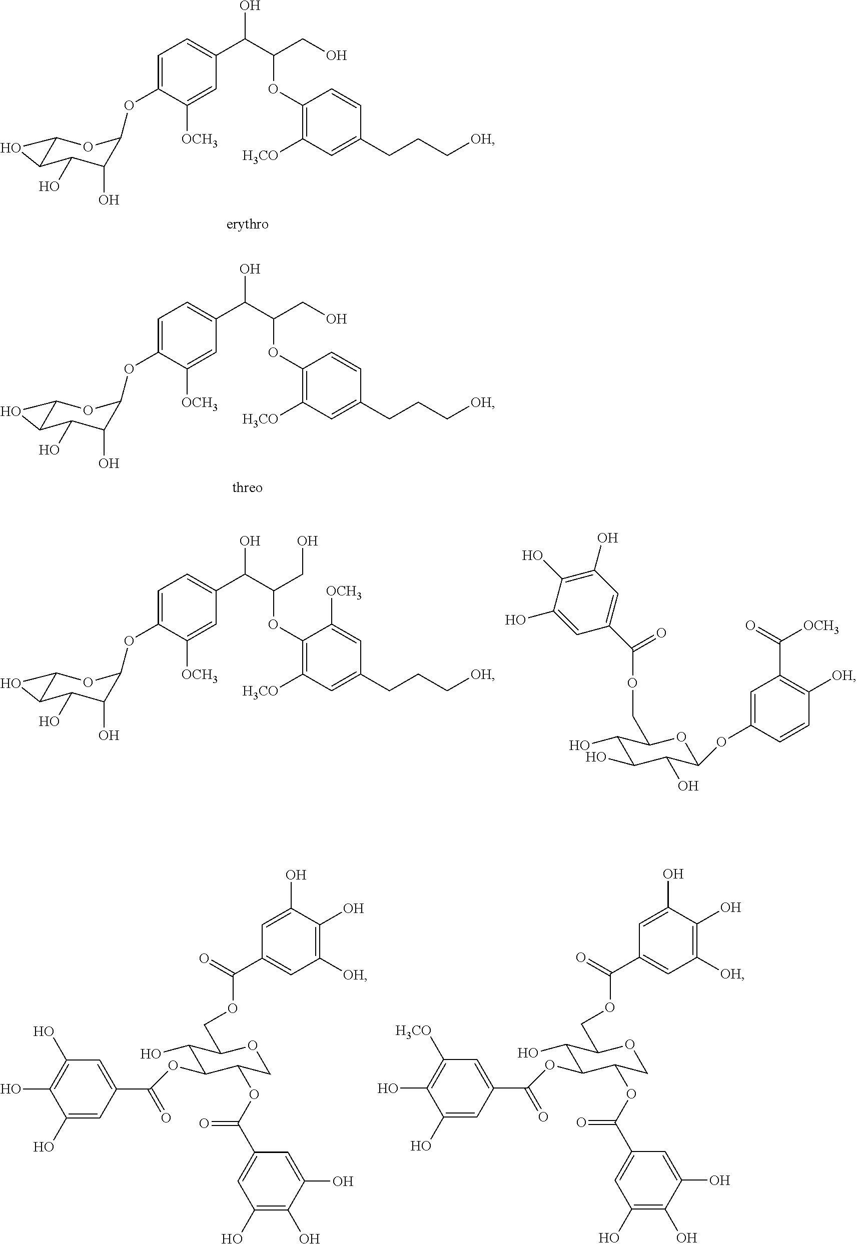 [SMILES: COc1cc(CCCO)ccc1OC(CO)C(O)c1ccc(OC2OC3OC3C(O)C2O)c(OC)c1 ; COc1cc(CCCO)ccc1OC(CO)C(O)c1ccc(OC2OC3OC3C(O)C2O)c(OC)c1 ; COc1cc(C(O)C(CO)Oc2c(OC)cc(CCCO)cc2OC)ccc1OC1OC2OC2C(O)C1O ; COC(=O)c1cc(OC2OC(COC(=O)c3cc(O)c(O)c(O)c3)C(O)C(O)C2O)ccc1O ; O=C(OCC1OCC(OC(=O)c2cc(O)c(O)c(O)c2)C(OC(=O)c2cc(O)c(O)c(O)c2)C1O)c1cc(O)c(O)c(O)c1 ; COc1cc(C(=O)OC2C(OC(=O)c3cc(O)c(O)c(O)c3)COC(COC(=O)c3cc(O)c(O)c(O)c3)C2O)cc(O)c1O]

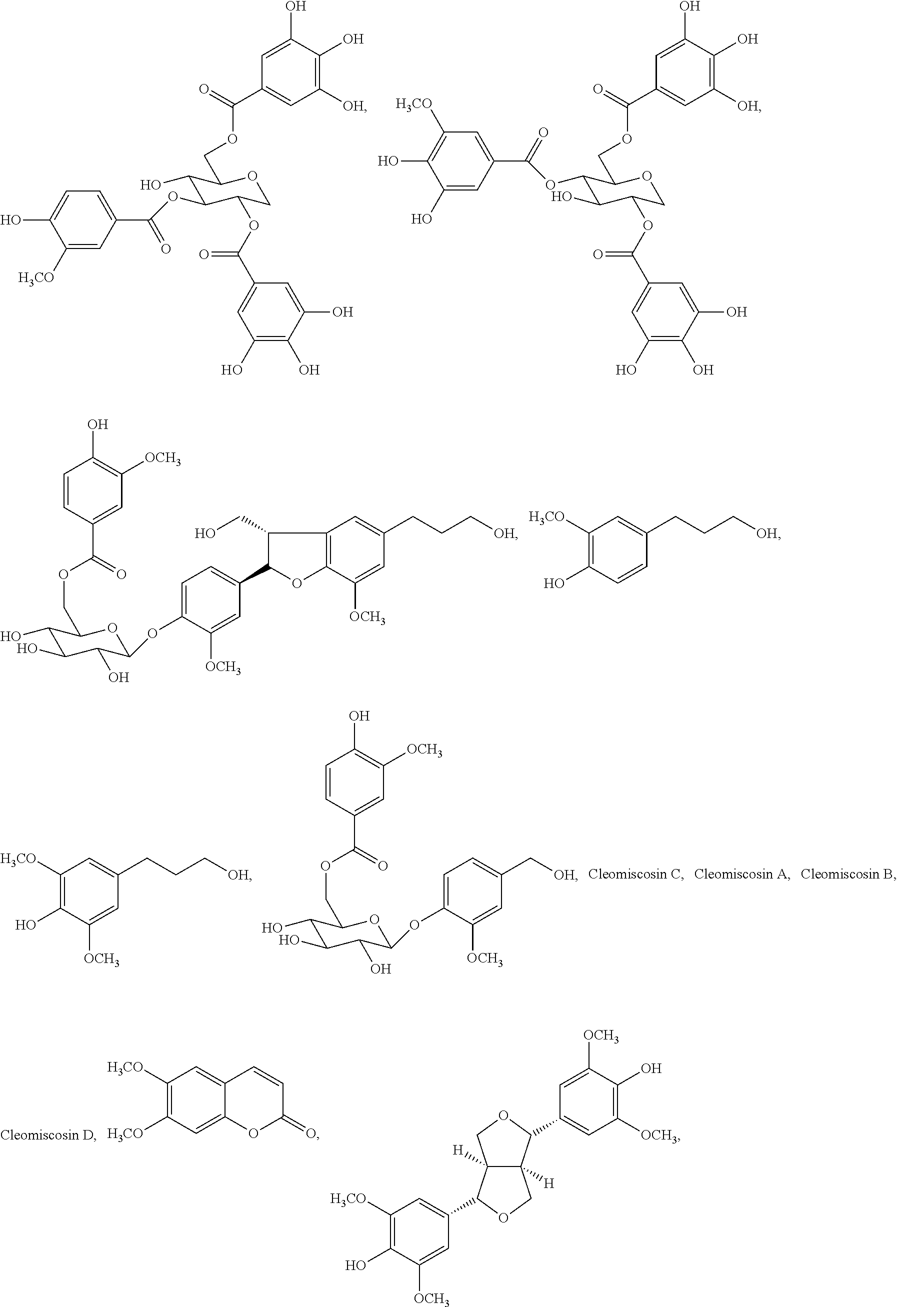

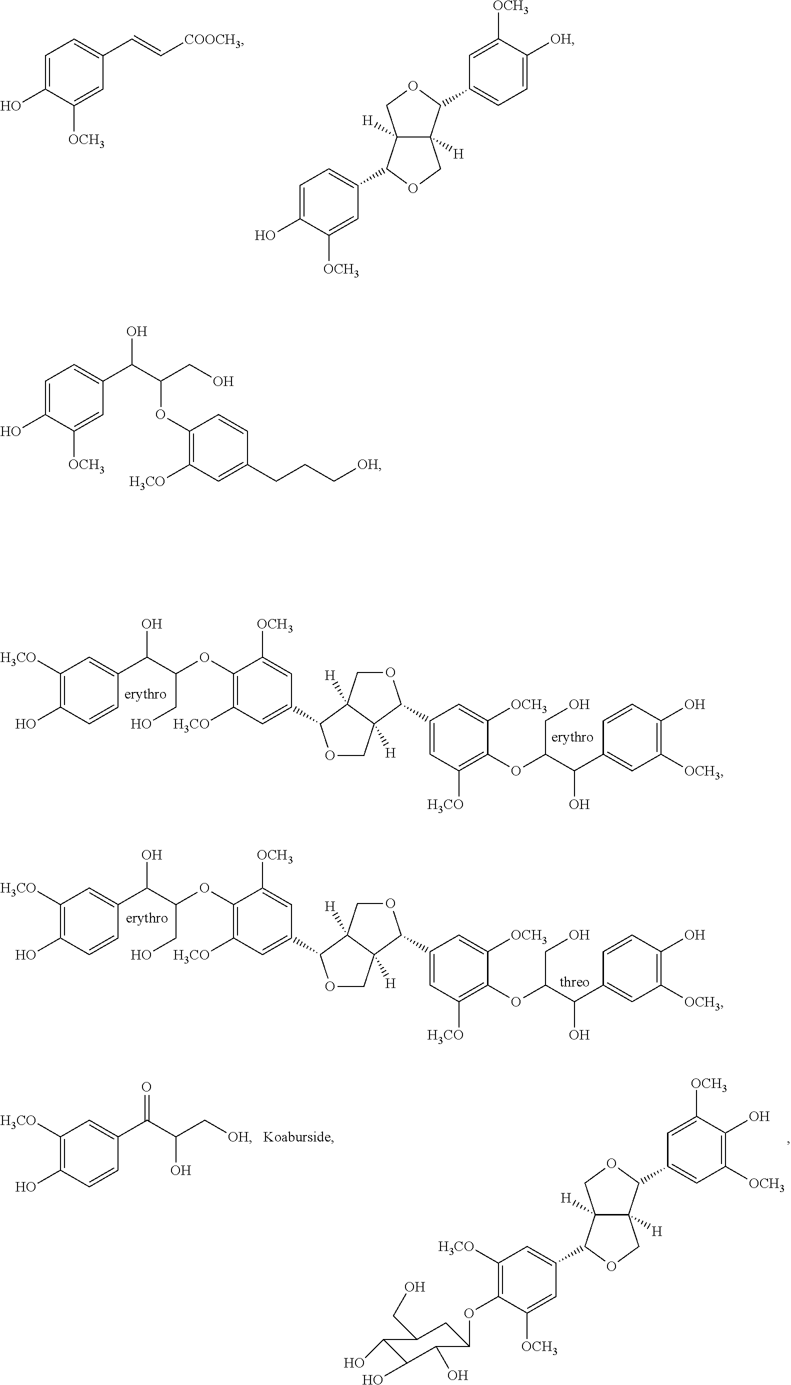

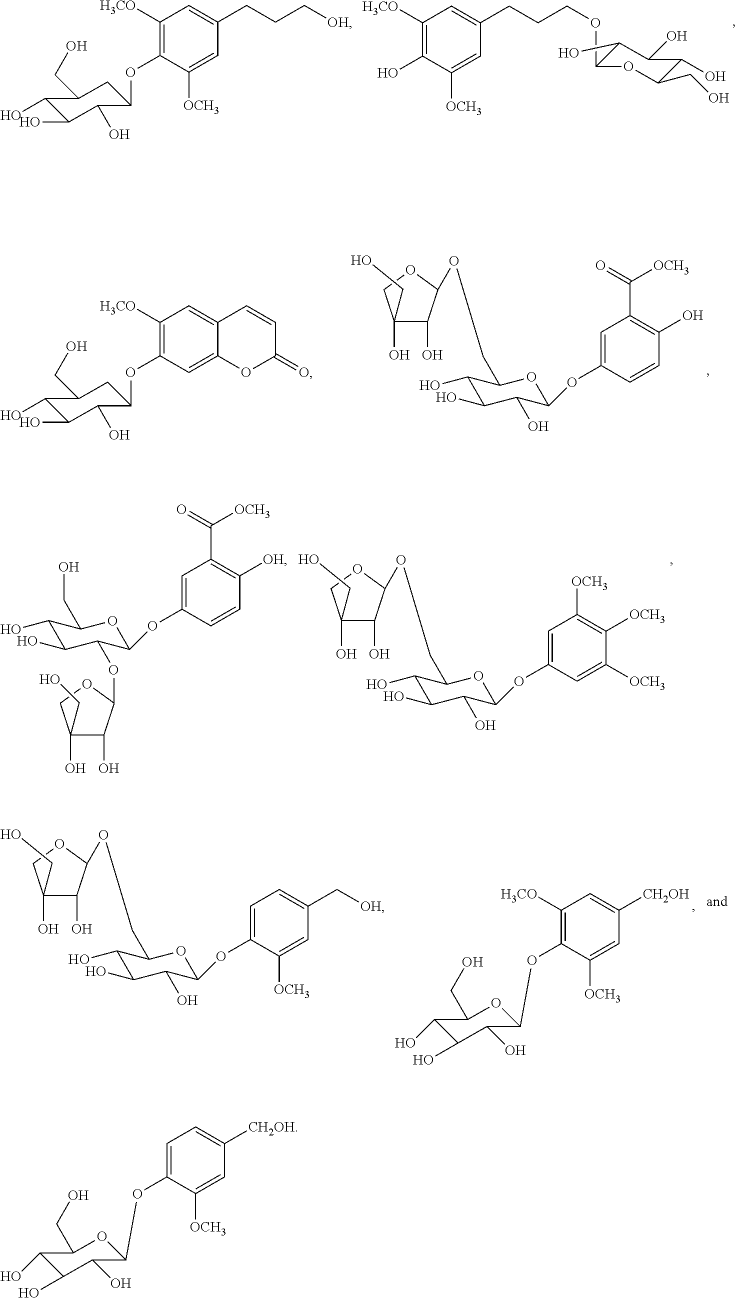

50. The sweetening composition according to claim 45, wherein the residue of syrup or syrup-derived product production comprises diatomaceous earth, celite, kieselguhr, silica, silicon dioxide, calcium, natural sugar sand, ground bones, slop, clay and the like.

51. The sweetening composition according to claim 45, wherein said at least one sweetener is chosen from a nutritive sweetener and a non-nutritive sweetener.

52. The sweetening composition according to claim 51, wherein said nutritive sweetener is at least one of honey, birch syrup, pine syrup, hickory syrup, poplar syrup, palm syrup, sugar beet syrup, sorghum syrup, corn syrup, cane syrup, golden syrup, barley malt syrup, a molasse, brown rice syrup, agave nectar, yacon syrup, fructose, maltitol, brown sugar, Okinawa syrup or combinations thereof.

53. The sweetening composition according to claim 51, wherein said non-nutritive sweetener is at least one of adenosine monophosphate, acesulfame potassium, alitame, aspartame, anethole, cyclamate, glycyrrhizin, lo han guo, miraculin, neotame, perillartine, saccharin, selligueain A, a Stevia rebaudiana extract, sucralose, thaumatin, neohesperdine DC, thavmatin, brazzein and inulin.

54. The sweetening composition according to claim 53, wherein said Stevia rebaudiana extract comprises at least one of stevioside, rebaudioside A, rebaudioside B, and rebaudioside C.

55. An infusion composition for the preparation of a beverage comprising:
an extract of an Acer tree leaf, samara fruit, samara seed, root, samara stem leaf stem, twig, heartwood, sapwood, a whole branch, a bark of a branch, a wood of a branch, and/or bark.

56. The infusion composition according to claim 55, further comprising a herbal component.

57. The infusion composition according to claim 56, wherein said herbal component at least one of a tea, and a herbal tea.

58. The composition according to claim 57, wherein said tea is at least one of Bai Hao Yinzhen tea, Bai Mu Dan tea, Pai Mu Tan tea, Gong Mei tea, Shou Mei tea, White Puerh tea, Ceylon White tea, Darjeeling White tea, Assam White tea, African White tea, Junshan Yinzhen tea, Huoshan Huangya tea, Meng Ding tea, Huangya tea, Da Ye Qing tea, Huang Tang tea, Junshan Yinzhen tea, Longjing tea, Hui Ming tea, Long Ding tea, Hua Ding tea, Qing Ding tea, Gunpowder tea, Bi Luo Chun tea, Rain Flower tea, Shui Xi Cui Bo tea, Camellia Sinensis tea, Yu Lu tea, Xin Yang Mao Jian tea, Chun Mee tea, Gou Gu Nao tea, Yun Wu tea, Da Fang tea, Huangshan Maofeng tea, Lu'An Guapian tea, Hou Kui tea, Tun Lu tea, Huo Qing tea, Wuliqing tea, Hyson tea, Zhu Ye Qing tea, Meng Ding Can Lu tea, Genmaicha tea, Gyokuro tea, Kabusecha tea, Sencha tea, Fukamushicha tea, Tamaryokucha tea, Bancha tea, Kamairicha tea, Kukicha tea, Mecha tea, Konacha tea, Matcha tea, Genmaicha tea, Bancha tea, Höjicha tea, Tencha tea, Aracha tea, Shincha tea, funmatsucha tea or combinations thereof.

59. The composition according to claim 58, wherein said herbal tea is at least one of anise tea, artichoke tea, roasted barley tea, bee balm tea, boldo tea, cannabis tea, catnip tea, Ilex causue leaves tea, cinnamon tea, coffee leaves tea, coffee cherry tea, Cerasse tea, dried chamomile blossoms tea, chrysanthemum tea, citrus peel tea, bergamot tea, orange peel tea, dandelion tea, dill tea, echinacea tea, essiac tea, fennel tea, gentian tea, ginger root tea, ginseng tea, hawthorn tea, hibiscus tea, rose hip tea, honeybush tea, horehound tea, hydrangea tea, Jiaogulan tea, Kapor tea, Kava root tea, Ku Ding tea, Labrador tea, Lapacho tea, lemon balm tea, lemon grass tea, licorice root tea, lime blossom tea, yerba mate tea, mate de coca tea, mint tea, european mistletoe tea, neem leaf tea, nettle leaf tea, asiatic pennywort leaf tea, pennyroyal leaf tea, pine tea, red raspberry leaf tea, scorched rice tea, rooibos tea, roselle petals, rosemary memory herb tea, sage tea, skullcap tea, serendib tea, sobacha, spicebush leaf tea, spruce tea, staghorn sumac fruit tea, stevia tea, St. John's Wort tea, tulsi tea, uncaria tomentosa, valerian tea, Verbena tea, vetiver tea, roasted wheat tea, wax gourd tea, Wong Logat tea, woodruff tea, yarrow tea, yerba mate tea, yuen kut lam kam wo tea or combinations thereof.

60. A method of infusing an infusion composition according to any one of claims 55-59, wherein said infusion composition is infused with a maple tree based matrix.

61. The method according to claim 60, wherein said maple tree based matrix is chosen from a maple tree sap, a concentrated maple tree water, a maple tree syrup.

62. A compound of formula (I), or a pharmaceutically acceptable salt thereof: 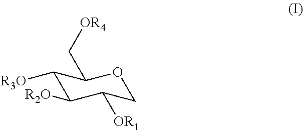
wherein R₁ and R₄ are each independently chosen from
(dd) C₁₋₃ alkyl substituted with 0-3 groups selected from halogen, —OH, —OCH₃,
(ee) —C(═O)H,
(ff) —C(═O)C₁₋₃ alkyl substituted with 0-3 groups selected from halogen, —OH, —OCH₃,
(gg) —CN,
(hh) —HC—NOH,
(ii) —(CH₃)C═NOH,
(jj) —HC═NOC₁₋₃ alkyl substituted with 0-3 groups selected from halogen, —OH, —OCH₃,
(kk) —(CH₃)C═NOC₁₋₃ alkyl substituted with 0-3 groups selected from halogen, —OH, —OCH₃,
(ll) —C(═O)OC₁₋₃alkyl substituted with 0-3 groups selected from halogen, —OH, —OCH₃,
(mm) aryl substituted with 0-5 group selected from halogen, —OH, —OCH₃,
(nn) —C(═O) aryl substituted with 0-5 group selected from halogen, —OH, —OCH₃,
(oo) HET-aryl wherein HET is a 5 or 6-membered heteroaromatic ring containing 1-3 heteroatoms selected from O, N and S, wherein said HET-aryl is substituted with 0-5 group selected from halogen, —OH, —OCH₃,
(pp) —C(═O) HET-aryl wherein HET is a 5 or 6-membered heteroaromatic ring containing 1-3 heteroatoms selected from O, N and S, wherein said HET-aryl is substituted with 0-5 group selected from halogen, —OH, —OCH₃,
(qq) cinnamon acyl substituted with 0-5 group selected from halogen, —OH, —OCH₃, and
R₂ and R₃ are each independently chosen from
(rr) C₁₋₃ alkyl substituted with 0-3 groups selected from halogen, —OH, —OCH₃,
(ss) —C(═O)H,
(tt) —-C(═O)C₁₋₃ alkyl substituted with 0-3 groups selected from halogen, —OH, —OCH₃,
(uu) —CN,
(vv) —HC—NOH,
(ww) —(CH₃)C═NOH,
(xx) —HC═NOC₁₋₃ alkyl substituted with 0-3 groups selected from halogen, —OH, —OCH₃,
(yy) —(CH₃)C═NOC₁₋₃alkyl substituted with 0-3 groups selected from halogen, —OH, —OCH₃,
(zz) —C(═O)OC₁₋₃alkyl substituted with 0-3 groups selected from halogen, —OH, —OCH₃,
(aaa) aryl substituted with 0-5 group selected from halogen, —OH, —OCH₃,
(bbb) —C(═O) aryl substituted with 0-5 group selected from halogen, —OH, —OCH₃,
(ccc) HET-aryl wherein HET is a 5 or 6-membered heteroaromatic ring containing 1-3 heteroatoms selected from O, N and S, wherein said HET-aryl is substituted with 0-5 group selected from halogen, —OH, —OCH₃,
(ddd) —C(═O) HET-aryl wherein HET is a 5 or 6-membered heteroaromatic ring containing 1-3 heteroatoms selected from O, N and S, wherein said HET-aryl is substituted with 0-5 group selected from halogen, —OH, —OCH₃,
(eee) cinnamon acyl substituted with 0-5 group selected from halogen, —OH, —OCH₃, and
(fff) —H.

63. A molecule consisting of:

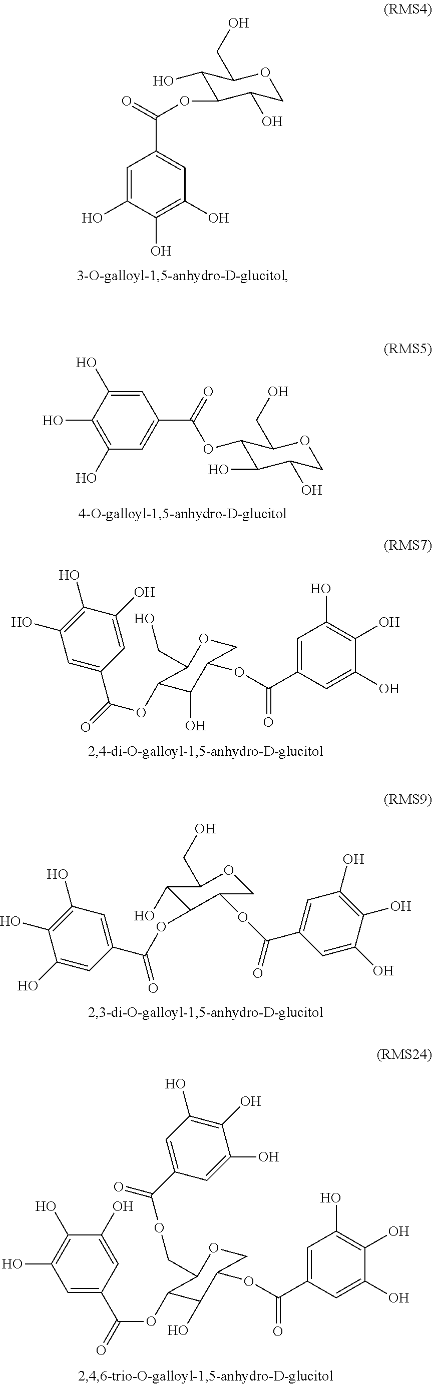 [SMILES: O=C(OC1C(O)COC(CO)C1O)c1cc(O)c(O)c(O)c1 ; O=C(OC1C(CO)OCC(O)C1O)c1cc(O)c(O)c(O)c1 ; O=C(OC1COC(CO)C(OC(=O)c2cc(O)c(O)c(O)c2)C1O)c1cc(O)c(O)c(O)c1 ; O=C(OC1COC(CO)C(O)C1OC(=O)c1cc(O)c(O)c(O)c1)c1cc(O)c(O)c(O)c1 ; O=C(OCC1OCC(OC(=O)c2cc(O)c(O)c(O)c2)C(O)C1OC(=O)c1cc(O)c(O)c(O)c1)c1cc(O)c(O)c(O)c1]

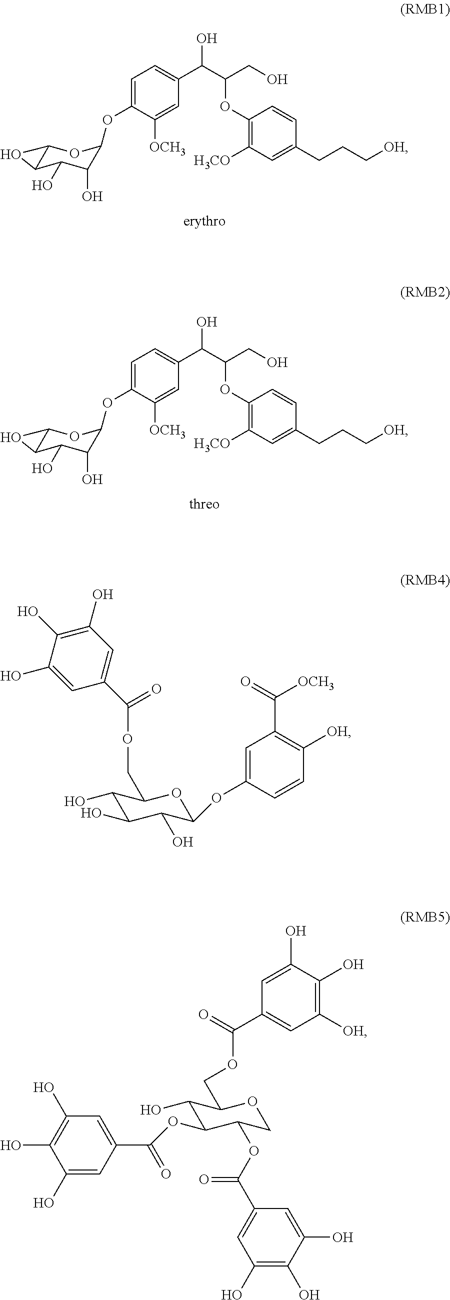 [SMILES: COc1cc(CCCO)ccc1OC(CO)C(O)c1ccc(OC2OC3OC3C(O)C2O)c(OC)c1 ; COc1cc(CCCO)ccc1OC(CO)C(O)c1ccc(OC2OC3OC3C(O)C2O)c(OC)c1 ; COC(=O)c1cc(OC2OC(COC(=O)c3cc(O)c(O)c(O)c3)C(O)C(O)C2O)ccc1O ; O=C(OCC1OCC(OC(=O)c2cc(O)c(O)c(O)c2)C(OC(=O)c2cc(O)c(O)c(O)c2)C1O)c1cc(O)c(O)c(O)c1]

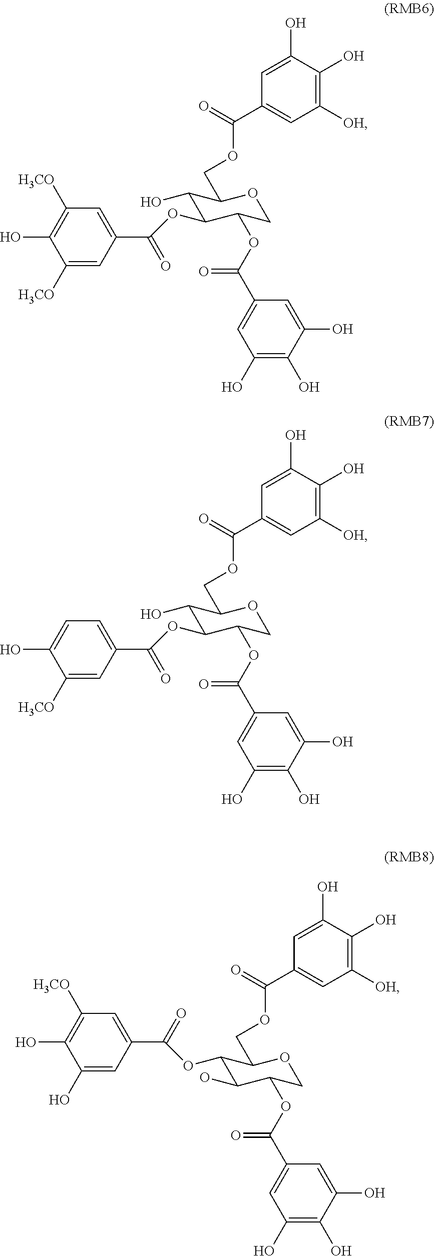 [SMILES: COc1cc(C(=O)OC2C(OC(=O)c3cc(O)c(O)c(O)c3)COC(COC(=O)c3cc(O)c(O)c(O)c3)C2O)cc(OC)c1O ; COc1cc(C(=O)OC2C(OC(=O)c3cc(O)c(O)c(O)c3)COC(COC(=O)c3cc(O)c(O)c(O)c3)C2O)ccc1O ; COc1cc(C(=O)OC2C(COC(=O)c3cc(O)c(O)c(O)c3)OCC(OC(=O)c3cc(O)c(O)c(O)c3)C2O)cc(O)c1O]

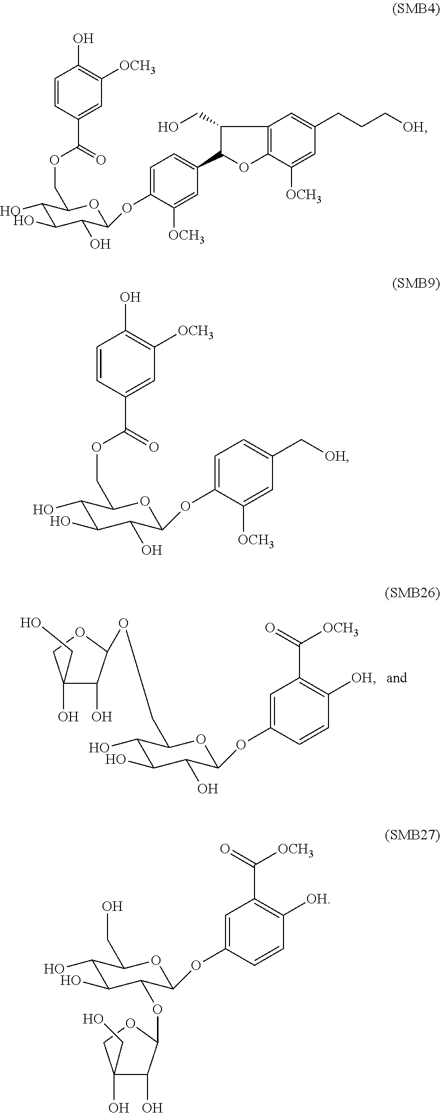

64. A method of treating an ailment comprising treating a subject with a therapeutically effective amount of a compound according to any one of claims 62-63.

65. The method according to claim 64, wherein said ailment is diabetes, a cancer, an arthritis, a micro-organism infection, a neurodegenerative disease, an inflammatory disease, an oxidative stress related disease, a heart disease, Alzheimer's diseases, a liver disorder a metabolic syndrome, a damaged hepatic function, a hepatic and liver dyslipidemia, a hepatitis, a liver cancer, an atherosclerosis, a hypertension, a skin disease, an eczema, and a psoriasis.

66. Use of a compound according to any one of claims 62-63 for the preparation of a medicament for the treatment of an ailment.

67. Use of a compound according to any one of claims 62-63 for the treatment of an ailment.

68. The use as claimed in any one of claims 101-102, wherein said ailment is chosen from a diabetes, a cancer, an arthritis, a micro-organism infection, a neurodegenerative disease, an inflammatory disease, an oxidative stress related disease, a heart disease, Alzheimer's diseases, a liver disorder a metabolic syndrome, a damaged hepatic function, a hepatic and liver dyslipidemia, a hepatitis, a liver cancer, an atherosclerosis, a hypertension, a skin disease, an eczema, and a psoriasis.
